(19) United States
(12) Patent Application Publication
(10) Pub. No.: US 2026/0337562 A1
(43) Pub. Date: Oct. 29, 2026

(54) IN VITRO GENERATED HEMATOPOIETIC STEM PROGENITOR CELLS AND T CELLS AND METHODS OF MAKING AND USING THE SAME

(71) Applicant: REGENTS OF THE UNIVERSITY OF MINNESOTA, MINNEAPOLIS, MN (US)

(72) Inventors: Beau R. WEBBER, Minneapolis, MN (US); Brett N. NAPIWOCKI, Minneapolis, MN (US); Lage VON DISSEN, Minneapolis, MN (US)

(21) Appl. No.: 19/107,853

(22) PCT Filed: Sep. 1, 2023
(86) PCT No.: PCT/US2023/073345

§ 371 Date: Feb. 28, 2025

Related U.S. Application Data
(60) Provisional application No. 63/403,055, filed on Sep. 1, 2022.

(51) Int. Cl. C12N 5/0783; A61K 35/12; A61K 35/17; A61K 35/28; A61K 40/11; A61K 40/42; A61K 40/46; A61P 35/00; C12N 5/0789

(52) U.S. Cl. CPC C12N 5/0636; A61K 35/17; A61K 35/28; A61K 40/11; A61K 40/42; A61K 40/46; A61P 35/00; C12N 5/0647; A61K 2035/124; C12N 2500/90; C12N 2501/115; C12N 2501/125; C12N 2501/155; C12N 2501/16; C12N 2501/165; C12N 2501/42; C12N 2501/58; C12N 2501/60; C12N 2506/45; C12N 2529/00

(57) ABSTRACT

Hematopoietic stem progenitor cells (HSPCs) are multipotent, self-renewing, progenitor cells capable of generating every type of blood cell arising from both the lymphoid and myeloid lineages. Since many types of cancer, including leukemia, lymphoma, and myeloma, affect the development, function, and viability of a number of blood cells, finding a means of producing HSPCs has become an important area of research. Disclosed are CD34+hematopoietic stem progenitor cells (HSPCs) generated from induced pluripotent stem cells (IPSCs) and T cells generated from IPSCs under minimal cytokine conditions, and methods of generating and using the same for the treatment of diseases and disorders.

### CROSS-REFERENCE TO RELATED APPLICATIONS

The present application claims priority to U.S. Provisional Patent Application No. 63/403,055 that was filed Sep. 1, 2022, the entire contents of which are hereby incorporated by reference.

### STATEMENT REGARDING FEDERALLY SPONSORED RESEARCH OR DEVELOPMENT

Not applicable.

### BACKGROUND

Hematopoietic stem progenitor cells (HSPCs) are multipotent, self-renewing, progenitor cells capable of generating every type of blood cell arising from both the lymphoid and myeloid lineages. Since many types of cancer, including leukemia, lymphoma, and myeloma, affect the development, function, and viability of a number of blood cells, finding a means of producing HSPCs has become an important area of research. Among other applications, HSPCs can be used in a transplant, for example, to repopulate the bone marrow following a course of radiation or chemotherapy, thus restoring the population and functionality of lymphocytes and other blood cells. However, one problem with HSPC transplants is the risk of immune rejection, such as graft-versus-host disease (GVHD) or bone marrow failure, following the transplant, particularly if the cells were derived from an allogeneic source. HLA matching can help to reduce this risk, but autologous transplantation is ideal.

In many cases, a patient does not have an adequate or viable population of blood or immune cells to expand ex vivo for subsequent transplantation. One way to overcome this challenge, as well as that of immune rejection, involves the use of induced pluripotent stem cells (iPSCs), whereby fully differentiated cells (such as fibroblasts, derived from the patient) are de-differentiated ex vivo through epigenetic reprogramming into iPSCs, through the use of various transcription factors (i.e., the so-called “Yamanaka factors”). Once iPSCs are generated, they can theoretically be re-differentiated into any cell type of interest, whether fully differentiated into primary immune cells, or partially differentiated into a multipotent stem cell (such as an HSPC).

Unfortunately, not all iPSCs will give rise to HSPCs, even if they are encouraged or constrained to undergo hematopoietic differentiation. This failure to produce HSPCs can be explained, in part, from the fact that there are at least two distinct branches or waves of hematopoiesis that occur during embryonic development. The first wave, deemed primitive hematopoiesis, is believed to produce primitive erythrocytes, macrophages, and megakaryocytes. The second wave, deemed definitive hematopoiesis, produces every myeloid and lymphoid cell type as well as HSPCs. Perhaps most importantly, it is only this latter definitive wave that produces cells that are capable of engraftment in the host.

In light of these facts, finding the cell signaling pathways and gene regulatory networks that promote definitive hematopoietic differentiation is crucial for producing an autologous source of HSPCs from patient-derived iPSCs. Thus, there is a need in the art for novel methods of generating HSPCs from patient-derived iPSCs.

### SUMMARY

In an aspect of the current disclosure, in vitro methods of generating CD34⁺ hematopoietic stem progenitor cells (HSPCs) from induced pluripotent stem cells (iPSCs) in minimal cytokine conditions are provided. In some embodiments, the methods comprise: (i) contacting iPSCs with one or more mesoderm differentiation factors selected from bone morphogenetic protein 4 (BMP4), activin A, and CHIR99021; (ii) subsequently contacting the cells of step (i) with one or more HSPC differentiation factors selected from basic fibroblast growth factor (bFGF), vascular endothelial growth factor (VEGF), stem cell factor (SCF), and SB431542; (iii) contacting the cells of step (ii) with a plurality of nucleic acids encoding one or more transcription factors selected from ETS-related gene (ERG), ligand-dependent corepressor (LCOR), homeobox A5 (HOXA5), homeobox A9 (HOXA9), and runx family transcription factor 1 (RUNX1); and (iv) contacting the cells of step (iii) with one or more HSPC differentiation factors selected from bFGF, VEGF, SCF, SB431542, and Y-27632 to generate CD34⁺ HSPCs.

In an aspect of the current disclosure, populations of CD34⁺ HSPCs are provided. In some embodiments, the populations are generated by a method of comprising: (i) contacting iPSCs with one or more mesoderm differentiation factors selected from bone morphogenetic protein 4 (BMP4), activin A, and CHIR99021; (ii) subsequently contacting the cells of step (i) with one or more HSPC differentiation factors selected from basic fibroblast growth factor (bFGF), vascular endothelial growth factor (VEGF), stem cell factor (SCF), and SB431542; (iii) contacting the cells of step (ii) with a plurality of nucleic acids encoding one or more transcription factors selected from ETS-related gene (ERG), ligand-dependent corepressor (LCOR), homeobox A5 (HOXA5), homeobox A9 (HOXA9), and runx family transcription factor 1 (RUNX1); and (iv) contacting the cells of step (iii) with one or more HSPC differentiation factors selected from bFGF, VEGF, SCF, SB431542, and Y-27632 to generate CD34⁺ HSPCs.

In an aspect of the current disclosure, populations of in vitro derived T cells are provided. In some embodiments, the populations of in vitro derived T cells are generated by contacting a population of HSPCs generated by a method comprising: (i) contacting iPSCs with one or more mesoderm differentiation factors selected from bone morphogenetic protein 4 (BMP4), activin A, and CHIR99021; (ii) subsequently contacting the cells of step (i) with one or more HSPC differentiation factors selected from basic fibroblast growth factor (bFGF), vascular endothelial growth factor (VEGF), stem cell factor (SCF), and SB431542; (iii) contacting the cells of step (ii) with a plurality of nucleic acids encoding one or more transcription factors selected from ETS-related gene (ERG), ligand-dependent corepressor (LCOR), homeobox A5 (HOXA5), homeobox A9 (HOXA9), and runx family transcription factor 1 (RUNX1); and (iv) contacting the cells of step (iii) with one or more HSPC differentiation factors selected from bFGF, VEGF, SCF, SB431542, and Y-27632 to generate CD34⁺ HSPCs; with one or more T cell differentiation factors selected from delta like canonical notch ligand 4 (DLL4) and vascular cell adhesion molecule (VCAM) to generate T cells.

In an aspect of the current disclosure, pharmaceutical compositions are provided. In some embodiments, the pharmaceutical compositions comprise a population of CD34⁺ hematopoietic stem progenitor cell (HSPCs) generated by a method comprising: (i) contacting iPSCs with one or more mesoderm differentiation factors selected from bone morphogenetic protein 4 (BMP4), activin A, and CHIR99021; (ii) subsequently contacting the cells of step (i) with one or more HSPC differentiation factors selected from basic fibroblast growth factor (bFGF), vascular endothelial growth factor (VEGF), stem cell factor (SCF), and SB431542; (iii) contacting the cells of step (ii) with a plurality of nucleic acids encoding one or more transcription factors selected from ETS-related gene (ERG), ligand-dependent corepressor (LCOR), homeobox A5 (HOXA5), homeobox A9 (HOXA9), and runx family transcription factor 1 (RUNX1); and (iv) contacting the cells of step (iii) with one or more HSPC differentiation factors selected from bFGF, VEGF, SCF, SB431542, and Y-27632 to generate CD34⁺HSPCs; and a pharmaceutically acceptable carrier.

In some embodiments, the pharmaceutical compositions comprise in vitro generated T cells generated by a method comprising: contacting a population of HSPCs generated by a method comprising: (i) contacting iPSCs with one or more mesoderm differentiation factors selected from bone morphogenetic protein 4 (BMP4), activin A, and CHIR99021; (ii) subsequently contacting the cells of step (i) with one or more HSPC differentiation factors selected from basic fibroblast growth factor (bFGF), vascular endothelial growth factor (VEGF), stem cell factor (SCF), and SB431542; (iii) contacting the cells of step (ii) with a plurality of nucleic acids encoding one or more transcription factors selected from ETS-related gene (ERG), ligand-dependent corepressor (LCOR), homeobox A5 (HOXA5), homeobox A9 (HOXA9), and runx family transcription factor 1 (RUNX1); (iv) contacting the cells of step (iii) with one or more HSPC differentiation factors selected from bFGF, VEGF, SCF, SB431542, and Y-27632 to generate CD34⁺HSPCs; with one or more T cell differentiation factors selected from delta like canonical notch ligand 4 (DLL4) and vascular cell adhesion molecule (VCAM) to generate T cells; and a pharmaceutically acceptable carrier.

In an aspect of the current disclosure, methods of treating a subject in need of reconstitution of hematopoietic stem progenitor cells (HPSCs) are provided. In some embodiments, the methods comprise: administering an effective amount of a pharmaceutical composition comprising a population of CD34⁺ hematopoietic stem progenitor cell (HSPCs) generated by a method comprising: (i) contacting iPSCs with one or more mesoderm differentiation factors selected from bone morphogenetic protein 4 (BMP4), activin A, and CHIR99021; (ii) subsequently contacting the cells of step (i) with one or more HSPC differentiation factors selected from basic fibroblast growth factor (bFGF), vascular endothelial growth factor (VEGF), stem cell factor (SCF), and SB431542; (iii) contacting the cells of step (ii) with a plurality of nucleic acids encoding one or more transcription factors selected from ETS-related gene (ERG), ligand-dependent corepressor (LCOR), homeobox A5 (HOXA5), homeobox A9 (HOXA9), and runx family transcription factor 1 (RUNX1); and (iv) contacting the cells of step (iii) with one or more HSPC differentiation factors selected from bFGF, VEGF, SCF, SB431542, and Y-27632 to generate CD34⁺ HSPCs; and a pharmaceutically acceptable carrier; to a subject in need thereof to reconstitute CD34⁺ HSPCs in the subject.

In an aspect of the current disclosure, methods of treating a subject in need of T cell adoptive transfer are provided. In some embodiments, the methods comprise: administering an effective amount of a pharmaceutical composition comprising in vitro generated T cells generated by a method comprising: contacting a population of HSPCs generated by a method comprising: (i) contacting iPSCs with one or more mesoderm differentiation factors selected from bone morphogenetic protein 4 (BMP4), activin A, and CHIR99021; (ii) subsequently contacting the cells of step (i) with one or more HSPC differentiation factors selected from basic fibroblast growth factor (bFGF), vascular endothelial growth factor (VEGF), stem cell factor (SCF), and SB431542; (iii) contacting the cells of step (ii) with a plurality of nucleic acids encoding one or more transcription factors selected from ETS-related gene (ERG), ligand-dependent corepressor (LCOR), homeobox A5 (HOXA5), homeobox A9 (HOXA9), and runx family transcription factor 1 (RUNX1); and (iv) contacting the cells of step (iii) with one or more HSPC differentiation factors selected from bFGF, VEGF, SCF, SB431542, and Y-27632 to generate CD34⁺ HSPCs; with one or more T cell differentiation factors selected from delta like canonical notch ligand 4 (DLL4) and vascular cell adhesion molecule (VCAM) to generate T cells; and a pharmaceutically acceptable carrier; to the subject as a T cell adoptive transfer therapy.

### BRIEF DESCRIPTION OF THE FIGURES

FIG. 1. Schematic of the iPSC-CD34 differentiation protocol with the D5TF LV added on Day 5.

FIG. 2. Day 7 of the iPSC-CD34 differentiation protocol. (A) GFP positive cells, (B) bright field image and (C) merged images. Scale bar=75 um.

FIG. 3. Brightfield images of the control normal differentiation and the D5TF conditions for the 1210 iPSC line. By Day 9~50% of the cells had died in the D5TF condition compared to the control condition that did not receive the lentivirus. Scale bar=250 um.

FIG. 4. Day 9 flow cytometry results for the 1210 iPSC line that completed the iPSC-CD34 differentiation protocol.

FIG. 5. Schematic of the iPSC-CD34 differentiation protocol using the Mirus TransIT®-mRNA Transfection Kit on Day 3.

FIG. 6. Bright field images of the control condition that did not receive any mRNA. Top scale bar=500 um, bottom scale bar=250 um.

FIG. 7. Bright field images of the 2.5 uL Mirus condition that received GFP mRNA on Day 3. Top scale bar=500 um, bottom scale bar =250 um

FIG. 8. Bright field images of the 5 uL Mirus condition that received GFP mRNA on Day 3. Top scale bar=500 um, bottom scale bar=250 um

FIG. 9. Bright field images and GFP expression in cells that received 2.5 or 5 uL of the Mirus kit. Scale bar=75 um.

FIG. 10. Schematic of iPSC-CD34 differentiation protocol with CD34+ cells magnetically selected on Day 5 and then re-seeded into new wells.

FIG. 11. Bright field images of CD34+ cells that were magnetically selected on Day 5 and re-seeded into a new well at a density of 20k CD 34+ cells/cm2 . Top scale bar is 500 um and the bottom scale bar is 250 um.

FIG. 12. Bright field images of CD34+ cells that were magnetically selected on Day 5 and re-seeded into a new well at a density of 30k CD 34+ cells/cm2 . Top scale bar is 500 um and the bottom scale bar is 250 um.

FIG. 13. Schematic of iPSC-CD34 differentiation protocol with all cells re-seeded on Day 4.

FIG. 14. All cells were accutased on Day 4 and re-seeded into new wells at different densities and then cultured for the remainder of the differentiation protocol.

FIG. 15. Flow cytometry graph plots demonstrating the control and different re-seeding conditions.

FIG. 16. Schematic of iPSC-CD34 differentiation protocol with all cells electroporated with GFP mRNA on Day 4 and then re-seeded into new wells and the differentiation continued until Day 9.

FIG. 17. GFP expression in the three different Neon electroporation conditions one day after electroporation. Scale bar=75 um.

FIG. 18. GFP expression determined by flow cytometry one day after electroporation.

FIG. 19. Schematic of iPSC-CD34 differentiation protocol with all cells electroporated with GFP mRNA or TF mRNA on Day 4 and then re-seeded into new wells and cultured until Day 9.

FIG. 20. iPSCs differentiated using the 2DMD protocol and electroporated with GFP or TF mRNA on Day 4.

FIG. 21. Flow cytometry plots demonstrating treatment with concentrated and unconcentrated TF mRNA.

FIG. 22. (A) Schematic of the experimental outline. (B) CD34 and CD43 expression one (Day 4+1), three (Day 4+3) and five (Day 4+5) days post electroporation for the three conditions tested. (C) Quantification of CD34+ and CD34+ CD43+ expression on Day 4+5 of differentiation.

FIG. 23. (A) CD34 and CD45 expression one (Day 4+1), three (Day 4+3) and five (Day 4+5) days post electroporation for the three conditions tested. (B) Quantification of CD34+CD45+ expression on Day 4+5 of differentiation.

FIG. 24. Colony forming unit assay using Day 9 iPSC-HPCs from the three conditions tested. Starting iPSC-CD34 input=10,000 cells.

FIG. 25. (A) Day 14 CD5 and CD7 expression for GFP and TF mRNA treated cells. (B) The % of CD7+cells and output of CD7+cells per CD34+input. (C) The % of CD5+CD7+cells and output of CD5+CD7+cells per CD34+input. * p<0.05, unpaired t-test.

FIG. 26. CD4, CD8 and CD3 expression 28 days into a feeder-free T cell differentiation.

FIG. 27. A cartoon depiction of the method described herein including extended differentiation from Day 9 to Day 13.

FIG. 28. (A) Bright field images of round, hematopoietic cells for the GFP and TF mRNA treated cells on Day 9 and 13 of differentiation. (B) Quantification of the amount of CD34+supernatant cells collected per well of a 6-well plate. Scale bar=250 um.

FIG. 29. Differences in CD34, CD43 and CD45 expression for Day 9 and Day 13 TF mRNA treated cells.

FIG. 30. CFU assay for Day 9 and Day 13 GFP and TF mRNA treated cells.

FIG. 31. (A) CD5 and CD7 expression and (B) CD7+ and CD5+ CD7+ yield after 14 days of a T cell differentiation.

FIG. 32. CD5, CD7 and CD3 expression 28 days into a feeder-free T cell differentiation.

FIG. 33. CD34 and CD43 expression for the 1210, AK and ZF iPSC lines differentiated into iPSC-HPCs.

FIG. 34. CD34 and CD45 expression for the 1210, AK and ZF iPSC lines differentiated into iPSC-HPCs.

FIG. 35. CD5 and CD7 expression after 14 Days in a feeder-free T cell differentiation for Day 9 and Day 13 iPSC-HPCs.

FIG. 36. CD5 and CD7 expression after 28 Days in a feeder-free T cell differentiation for Day 9 and Day 13 iPSC-HPCs.

FIG. 37. CD4 and CD8 expression after 28 Days in a feeder-free T cell differentiation for Day 9 and Day 13 iPSC-HPCs.

FIG. 38. CD3 expression after 28 Days in a feeder-free T cell differentiation for Day 9 and Day 13 iPSC-HPCs.

FIG. 39. (A) Total fold expansion and (B) cell viability for 1210 iPSC-HPCs cultured on LDCM or DLL4/VCAM for 14 days of a T cell differentiation.

FIG. 40. CD5 and CD7 expression of 1210 iPSC-HPCs cultured for 14 and 28 days on LDCM or DLL4/VCAM in a T cell differentiation.

FIG. 41. CD56 and CD38 expression of 1210 iPSC-HPCs cultured for 14 and 28 days on LDCM or DLL4/VCAM in a T cell differentiation.

FIG. 42. CD3 expression of 1210 iPSC-HPCs cultured for 14 and 28 days on LDCM or DLL4/VCAM in a T cell differentiation.

FIG. 43. CD4 and CD8 expression of 1210 iPSC-HPCs cultured for 14 and 28 days on LDCM or DLL4/VCAM in a T cell differentiation.

FIG. 44. Schematic of three different differentiation protocols (All cytokine, bFGF, and No cytokine) using different cytokine combinations. All cytokine conditions include VEGF, SCF, IL-3, IL-6, TPO, and SR-1 in the culture starting at day 5. bFGF conditions include bFGF in culture starting at day 5, without IL-3, IL-6, TPO or SR-1. No cytokine conditions do not include the use bFGF, IL-3, IL-6, TPO, SR-1 in culture.

FIG. 45. 2DMDLvD6 Developmental Panel. Flow cytometry plots showing SSC vs CD117 (c-kit) on A) day 7 (top) and day 9 (bottom) and B) day 9 (top) day 11 (middle) and day 13 (bottom) row in the three differentiation protocol groups. All cytokine conditions include VEGF, SCF, IL-3, IL-6, TPO, and SR-1 in the culture starting at day 5. bFGF conditions include bFGF in culture starting at day 5, without IL-3, IL-6, TPO or SR-1. No cytokine conditions do not include the use bFGF, IL-3, IL-6, TPO, SR-1 in culture.

FIG. 46. Determine optimal plate coating. A) Experimental schematic. On day 9 or 13 differentiated cells were seeded onto plates coated with either LDCM or DLL4/VCAM. Lymphocyte differentiation and B) live cell counts, C) % and D) number of CD5+CD7+cells at day 9 and 13 on LDCM (left) or DLL4/VCAM (right) coated plates. All cytokine conditions include VEGF, SCF, IL-3, IL-6, TPO, and SR-1 in the culture starting at day 5. bFGF conditions include bFGF in culture starting at day 5, without IL-3, IL-6, TPO or SR-1. No cytokine conditions do not include the use bFGF, IL-3, IL-6, TPO, SR-1 in culture.

FIG. 47. T cell developmental Panel. CD38 and CD45 expression at day 14 from A) LDCM coated plates B) DLL4/VCAM coated plates and C) DLL4/VCAM coated plates at day 28. All cytokine conditions include VEGF, SCF, IL-3, IL-6, TPO, and SR-1 in the culture starting at day 5. bFGF conditions include bFGF in culture starting at day 5, without IL-3, IL-6, TPO or SR-1. No cytokine conditions do not include the use bFGF, IL-3, IL-6, TPO, SR-1 in culture.

FIG. 48. Determine optimal plate coating. CD5 and CD7 expression at day 14 from A) LDCM coated plates, B) DLL4/VCAM coated plates. All cytokine conditions include VEGF, SCF, IL-3, IL-6, TPO, and SR-1 in the culture starting at day 5. bFGF conditions include bFGF in culture starting at day 5, without IL-3, IL-6, TPO or SR-1. No cytokine conditions do not include the use bFGF, IL-3, IL-6, TPO, SR-1 in culture.

FIG. 49. Determine optimal plate coating for lymphocytes. CD8 and CD4 expression at day 14 from A) LDCM coated plates, B) DLL4/VCAM coated plates and day 28 on C) DLL4/VCAM coated plates. All cytokine conditions include VEGF, SCF, IL-3, IL-6, TPO, and SR-1 in the culture starting at day 5. bFGF conditions include bFGF in culture starting at day 5, without IL-3, IL-6, TPO or SR-1. No cytokine conditions do not include the use bFGF, IL-3, IL-6, TPO, SR-1 in culture.

FIG. 50. Determine optimal plate coating. CD3 and SSC expression at day 28 from A) DLL4/VCAM coated plates on day 14, B) LDCM coated plates on day 28 and C) DLL4/VCAM coated plates on day 28. All cytokine conditions include VEGF, SCF, IL-3, IL-6, TPO, and SR-1 in the culture starting at day 5. bFGF conditions include bFGF in culture starting at day 5, without IL-3, IL-6, TPO or SR-1. No cytokine conditions do not include the use bFGF, IL-3, IL-6, TPO, SR-1 in culture.

FIG. 51. Myeloid potential of day 9 or day 13 HSPC A) Experimental Schematic B) CFU Assay 14 days after seeding. All cytokine conditions include VEGF, SCF, IL-3, IL-6, TPO, and SR-1 in the culture starting at day 5. bFGF conditions include bFGF in culture starting at day 5, without IL-3, IL-6, TPO or SR-1. No cytokine conditions do not include the use bFGF, IL-3, IL-6, TPO, SR-1 in culture.

FIG. 52. Schematic of experimental timeline for iPSC-HPC differentiation. iPSCs are cultured on Geltrex in the presence of differentiation factors. On Day 4 cells are removed from the wells via accutase treatment and electroporated with either GFP-mRNA or TFmRNA. The cells are seeded back into new Geltrex-coated wells wells in the presence of Y-27632 and differentiation factors. On Day 5 cells are fed with either Minimal Cytokine or All Cytokine media. Supernatant cells consisting of CD34+ hematopoietic progenitor cells are collected from the wells on Day 13.

FIG. 53. Cell count and viability during iPSC-HPC differentiation. (a-b) Cell counts and viability for all cells in 12-well format and (c-d) supernatant cells in 6-well format. p*<0.05, **p<0.005

FIG. 54. iPSC-HPC flow cytometry characterization of adherent and supernatant cells. (a) CD34/CD43, CD34/CD45, CD34/CD117 expression for all cells on Day 13. Expression of iPSC-HPC markers during differentiation for the (b) adherent and supernatant cells and (c) Day 13 supernatant iPSC-HPCs. *p<0.05, **p<0.005, ***p<0.001, ****p<0.0001.

FIG. 55. Day 13 iPSC-HPC functional characterization. (a) Bright field images of Day 13 culture, (b) clonogenic output and (c) representative images from CFU assay. Scale bar =500 μm and 1000 μm for a and c, respectively. * p<0.05

FIG. 56. 14 Day T cell differentiation of iPSC-HPCs. (a) CD5/CD7 expression pre-gated on CD45+ and (b) quantification of output relative to starting iPSC-HPC input. *p<0.05, **p<, 0.005, ***p<0.001

FIG. 57. 28 Day T cell differentiation of iPSC-HPCs. (a) CD4/CD8 and TCRαβ/CD3 expression pre-gated on CD45+. (b) Expression of T cell markers during differentiation and total amount of live cells. (c) Frequencies of Vβ family genes determined by sequencing of the TCRβ CDR3 region. *p<0.05, **p<, 0.005, ***p<0.001

FIG. 58. tSNE analysis of Day 28 iPSC-T cells and primary control cells. (a) tSNE plots of primary cells and the 4 conditions clustered into groups using FlowSOM. (b) Expression and frequency of each condition based off cell surface expression. *p<0.05, **p<0.005

### DETAILED DESCRIPTION

Cell therapies are being used to treat a myriad of diseases. However, given that many patients do not have an adequate or viable population of blood or immune cells to expand ex vivo for subsequent transplantation for treating disease, alternative ways of producing cells are needed. One way to overcome this challenge, as well as that of immune rejection, involves the use of induced pluripotent stem cells (iPSCs), whereby fully differentiated cells are de-differentiated ex vivo through genetic reprogramming. Unfortunately, not all iPSCs will give rise to HSPCs.

In the present disclosure, the inventors have found signaling factors that can be incorporated into iPSCs to allow for differentiation into HSPCs at higher efficiency and in minimal cytokine conditions. This allows for the ability to produce a large population of CD34+ HSPCs that can be used for clinical applications. Further, these CD34+ HSPCs can be further differentiated into T cells for use in clinical treatments, including for treating cancers.

### Methods for Generating Hematopoietic Stem Progenitor Cells (HSPCs) From Induced Pluripotent Stem Cells (iPSCs)

The inventors discovered that mRNA coding for specific transcription factors, i.e., ERG, LCOR, HOXA5, HOXA9, and RUNX1, when transfected into differentiating iPSCs at a particular time point, i.e., about day 4 after the start of culture, promote definitive hematopoiesis and thereby generate HSPCs with a higher capacity for engraftment into a patient. Since these cells can be derived from an abundant source stemming from the patient, e.g., fibroblasts reprogrammed into iPSCs, the disclosed methods will provide a valuable cellular therapeutic tool to decrease the chance of immune rejection and to improve overall clinical outcomes for subjects requiring a transplant.

Accordingly, in one aspect of the current disclosure, methods of generating CD34⁺ hematopoietic stem progenitor cells (HSPCs) from induced pluripotent stem cells (iPSCs) in vitro are provided under minimal cytokine conditions. In some embodiments cells are differentiated on a solubilized basement membrane preparation. In some embodiments, the methods additionally comprise: (i) contacting iPSCs with one or more mesoderm differentiation factors selected from bone morphogenetic protein 4 (BMP4), activin A, and CHIR99021; (ii) subsequently contacting the cells of step (ii) with one or more HSPC differentiation factors selected from basic fibroblast growth factor (bFGF), vascular endothelial growth factor (VEGF), stem cell factor (SCF), and SB431542; (iii) contacting the cells of step (iii) with a plurality of nucleic acids encoding one or more transcription factors selected from ETS-related gene (ERG), ligand-dependent corepressor (LCOR), homeobox A5 (HOXA5), homeobox A9 (HOXA9), and runx family transcription factor 1 (RUNX1); (iv) contacting the cells of step (iii) with one or more HSPC differentiation factors selected from bFGF, VEGF, SCF, SB431542, and Y-27632 to generate CD34⁺ HSPCs.

As used herein, “hematopoietic stem progenitor cells (HSPCs)” may be used interchangeably with “hematopoietic progenitor cells (HPCs).” HSPCs may comprise hematopoietic stem cells (HSCs), which are pluripotent and have the capacity for self-renewal, and hematopoietic progenitor cells (HPCs) which, in an adult, are differentiated from HSCs and are the progenitor to all hematopoietic cells. As will be understood by one of skill in the art, HSCs are capable of engraftment into a subject, e.g., a subject in need of hematopoietic stem cell transplant, while HPCs have limited or no engraftment potential. However, HPCs are responsible for the generation of all hematopoietic cell lineages, i.e., myeloid and lymphoid lineages.

Methods of generating CD34⁺ HSPCs from iPSCs in vitro under culture conditions containing minimal cytokines are described herein. Minimal cytokine conditions may comprise HSPC differentiation or growth factors including bFGF. Minimal cytokine conditions may comprise additional factors including VEGF, SCF, SB431542, and Y-27632. Minimal cytokine conditions may comprise any of the differentiation factors listed here alone or in any combination. Minimal cytokine conditions may also comprise nucleic acids encoding one or more transcription factors as described herein. Minimal cytokine conditions do not include addition of exogenously added IL-3, IL-6, thrombopoietin (TPO), erythropoietin (EPO), aryl hydrocarbon receptor antagonists, e.g., StemRegenin1 (SR1), Fms-related tyrosine kinase 3 ligand (Flt3) alone or in any combination. Specifically, minimal cytokine conditions do not include IL-3, minimal cytokine conditions do not include IL-6, minimal cytokine conditions do not include TPO, minimal cytokine conditions do not include EPO, minimal cytokine conditions do not include SR1 and minimal cytokine conditions do not include Flt3. Minimal cytokine conditions may be called “bFGF-only”, “bFGF”, “No cytokine”, “minimal cytokine” or “cytokine-free” may be used interchangeably herein. Cells cultured under minimal cytokine conditions may be further differentiated into additional cell types, for example cell types of myeloid or lymphoid lineage.

HSPCs may be supported in culture by an extracellular matrix. The matrix can be deposited by preculturing and lysing a matrix-forming cell line (WO 99/20741), such as the STO mouse fibroblast line (ATCC Accession No. CRL-1503), or human placental fibroblasts. The matrix can also be coated directly into the culture vessel with isolated matrix components. suitable extracellular matrix components may include laminin, fibronectin, proteoglycan, entactin, heparan sulfate, and so on, alone or in various combinations. Substrates that can be tested using the experimental procedures described herein include not only other extracellular matrix components, but also polyamines, and other commercially available coatings. The extracellular matrix may be Matrigel®, Cultrex®, or JellaGel®. The extracellular matrix may be Geltrex®.

iPSC differentiation as described herein may also be supported by contacting the iPSC cells with member of the transforming growth factor B superfamily. In particular embodiments, the disclosed methods include contacting iPSC cells with bone morphogenetic protein 4 (BMP4) and Activin A.

iPSC differentiation as described herein may also be supported by inhibitors of glycogen synthase kinase 3 (GSK3) and WNT pathway activators. GSK3 inhibitors may have a diverse mechanism of action, including ATP-competitive inhibitors, non-ATP-competitive inhibitors or substrate-competitive inhibitors. In some instances, the GSK3 inhibitor is an aminopyrimidine. In particular, the GSK inhibitor may be CHIR99021.

iPSCs may be contacted by BMP4, Activin A and/or CHIR99021 for about 2 days.

The disclosed methods further include contacting iPSCs undergoing differentiation with growth factors to induce mesoderm differentiation. By way of example, and not limitation, these growth factors may include basic fibroblast growth factor (bFGF), vascular endothelial growth factor (VEGF) and/or stem cell factor (SCF).

Mesoderm differentiation as described herein may also be supported by inhibitors of the activin receptor-like kinase receptors, transforming growth factor β (TGFβ) ALK5, ALK4, and ALK7. In particular the inhibitor may be SB431542.

Mesoderm differentiation factors may be in contact with iPSCs undergoing differentiation for about 2 days.

The inventors contacted iPSCs undergoing differentiation with the nucleic acids encoding one or more transcription factors selected from ETS-related gene (ERG), ligand-dependent corepressor (LCOR), homeobox A5 (HOXA5), homeobox A9 (HOXA9), and runx family transcription factor 1 (RUNX1) of step (iii) of the disclosed methods, such that the nucleic acids were delivered to the cells, by electroporating mRNA into the cells. However, it is to be understood that alternative methods of delivery of nucleic acids are contemplated by the inventors including, but not limited to, transfection, particle bombardment, microinjection, sonoporation, photoporation, magnetofaction, and hydroporation.

In some embodiments, the iPSCs undergoing differentiation are electroporated at about 1000 V, about 1050V, about 1100 V, about 1150 V, about 1200 V, about 1250 V, about 1300 V, about 1350 V, about 1400V, about 1450 V, about 1500 V, about 1550 V, about 1600 V, about 1650 V, about 1700V, about 1750 V, about 1800 V, about 1850 V, about 1900 V, about 1950 V, or about 2000 V for about 5 ms, about 6 ms, about 7 ms, about 8 ms, about 9 ms, about 10 ms, about 11 ms, about 12 ms, about 13 ms, about 14 ms, or about 15 ms. In some embodiments, the cells are electroporated at about 1200 V for about 10 ms, or about 1450 V for about 10 ms, or about 1850 V for about 10 ms. In some embodiments, the cells are electroporated at about 1450 V for about 10 ms.

Following the introduction of transcription factors into the iPSCs, the iPSCs may be continued to be exposed to bFGF, VEGF and/or SCF for another 24 hrs, or longer or optionally, for the duration of the method. Additionally, SB43152 and Y-27632 may in in contact with the cell for approximately an additional 24 hours.

Cellular differentiation as described herein may also be supported by Rho-kinase inhibitors. In particular, the Rho-kinase inhibitor may be Y-27632.

In some embodiments, cellular differentiation as described herein may also be supported by antagonists of the aryl hydrocarbon receptor. In particular, the antagonist may be SR-1.

In some embodiments, the disclosed methods are performed using a concentration of about 5 ng/ml to about 100 ng/ml of BMP 4 , e.g., about 5 ng/ml, about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml, a concentration of about 5 ng/ml to about 50 ng/ml of activin A, e. g, about 5 ng/ml, about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, a concentration of about 0.5 μM to about 3 μM of CHIR99021, e.g., about 0.5 ng/ml, about 1.0 ng/ml, about 1.5 ng/ml, about 2.0 ng/ml, about 2.5 ng/ml, about 3.0 ng/ml, a concentration of about 5 ng/ml to about 100 ng/ml of bFGF, e.g., about 5 ng/ml, about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml, a concentration of about 5 ng/ml to about 100 ng/ml of VEGF, e.g., about 5 ng/ml, about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml, a concentration of about 5 ng/ml to about 100 ng/ml of SCF, e.g., about 5 ng/ml, about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml, a concentration of about 3μM to about 20μM of SB431542, e.g., about 3 μM, about 4 μM, about 5 μM, about 6 μM, about 7 μM, about 8 μM, about 9 μM, about 10 μM, about 11 μM, about 12μM, about 13 μM, about 14 μM, about 15 μM, about 16 μM, about 17 μM, about 18 μM, about 19 μM, about 20 μM, a concentration of about 5 μM to about 15 μM of Y-27632, e.g., about 5 μM, about 6 μM, about 7 μM, about 8 μM, about 9 μM, about 10 μM, about 11 μM, about 12 μM, about 13 μM, about 14 μM, about 15 μM, a concentration of about 5 ng/ml to about 50 ng/ml of IL-3, e.g., about 5 ng/ml, about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, a concentration of about 5 ng/ml to about 100 ng/ml of IL-6, e.g., about 5 ng/ml, about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml, a concentration of about 5 ng/ml to about 100 ng/ml of TPO, e.g., about 5 ng/ml, about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml, a concentration of about 0.5 μM to about 3 μM of SR-1, e.g., about 0.5 μM, about 1.0 μM, about 1.5 μM, about 2.0 μM, about 2.5 μM, about 3.0 μM.

Differentiation of HSPCs as described herein may optionally include the use of cytokines. In particular, interleukin (IL)-3, IL-6, UM171 and/or thrombopoietin (TPO).

Differentiation of HSPCs as described herein may further include the use of ligands contacted to the plate in which the cells are differentiated. These ligands may include delta-like 4 (DLL4), vascular cell adhesion protein 1 (VCAM), and/or StemSpan™ Lymphoid Differentiation Coating Material.

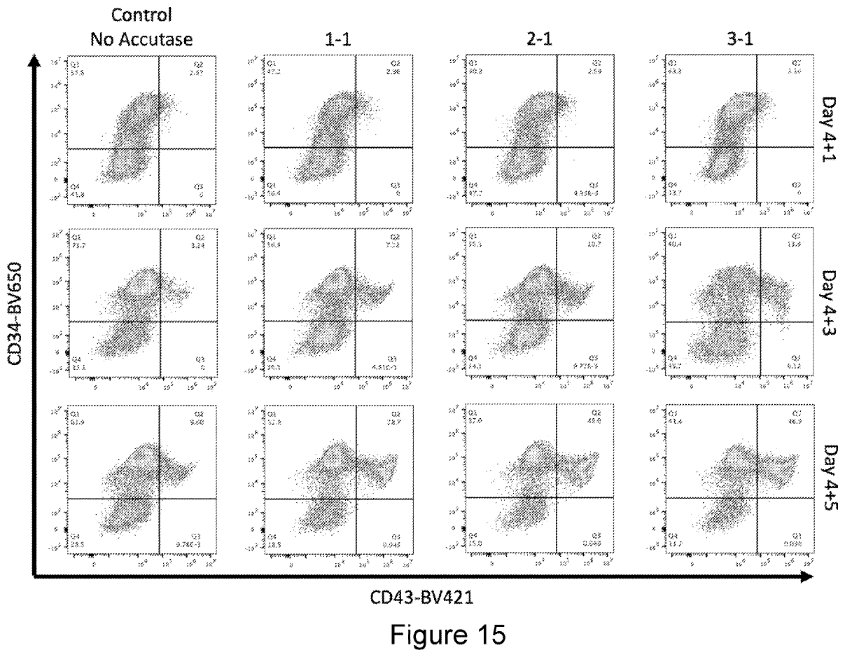

In some embodiments, the HSPCs generated by the disclosed methods express the marker CD34 and optionally the marker CD43 (FIG. 15). Detection of markers, e.g., CD34 or CD43, on cells may be performed by methods known in the art, e.g., flow cytometry, immunofluorescence, etc.

In some embodiments, the CD34⁺ HSPCs are isolated. Isolation of cells by using surface markers, e.g., CD34, may be performed by methods known in the art, e.g., isolation with magnetic beads, fluorescence-activated cell sorting (FACS), and the like.

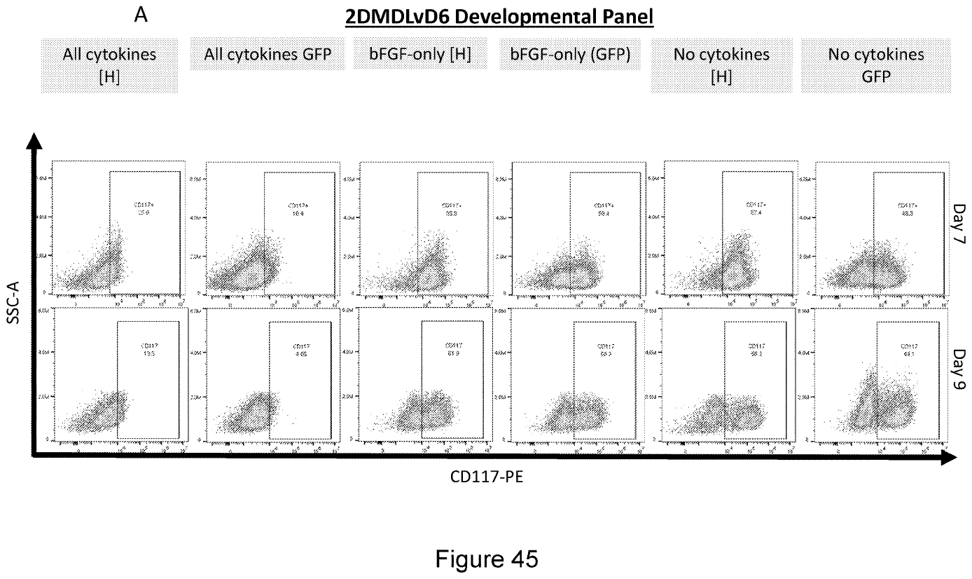

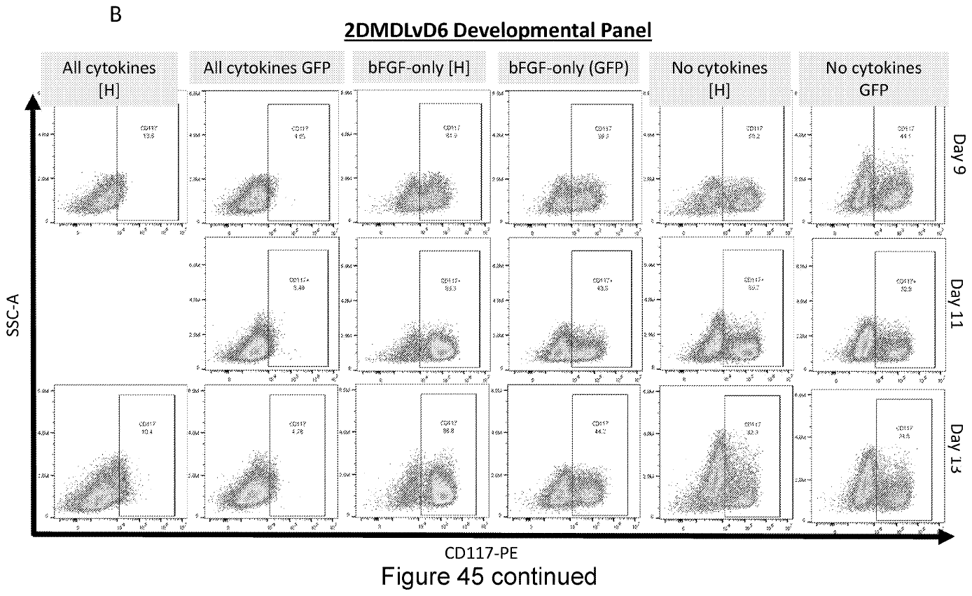

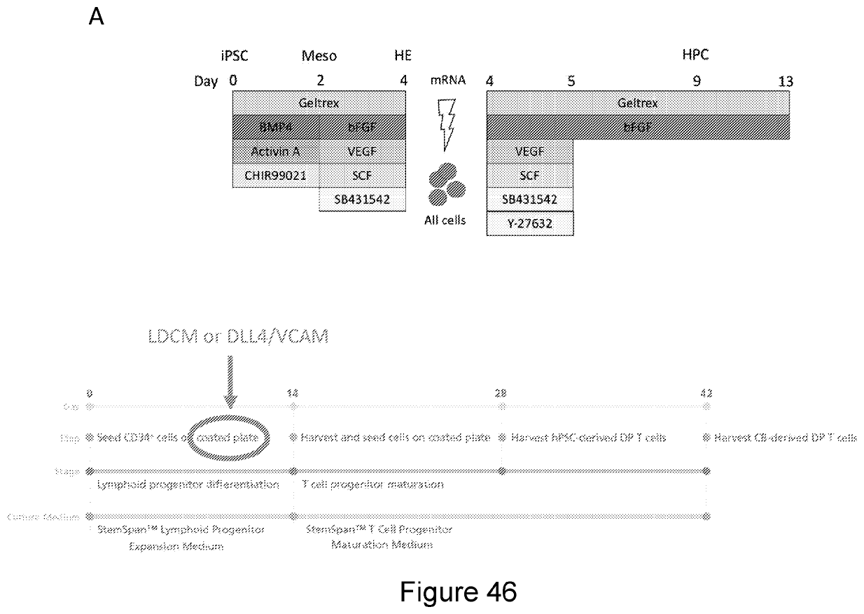

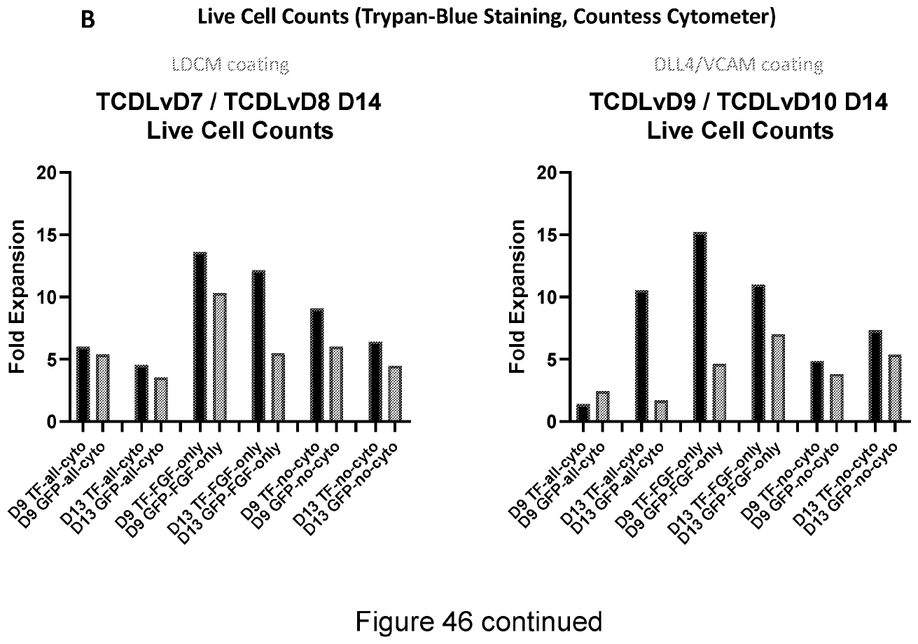

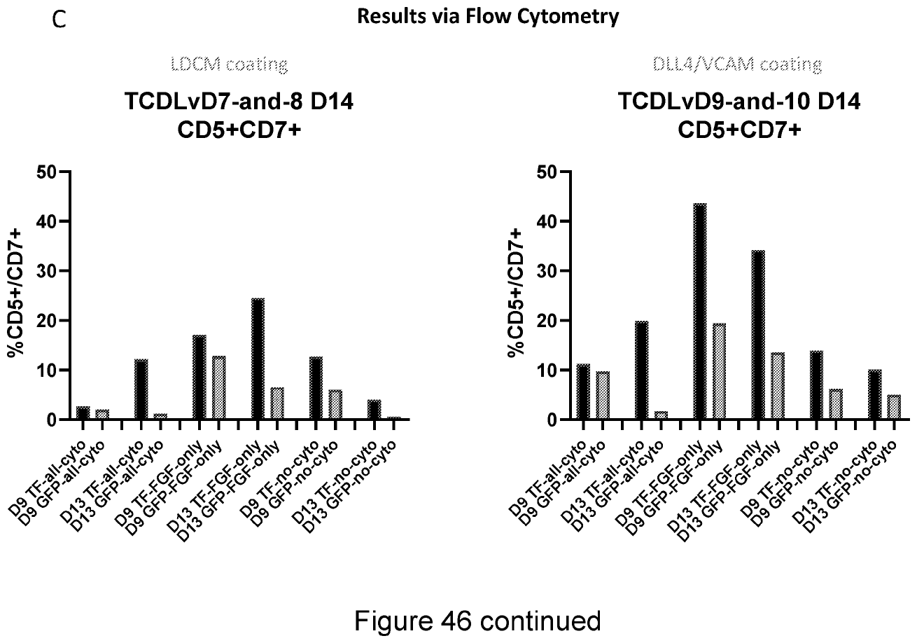

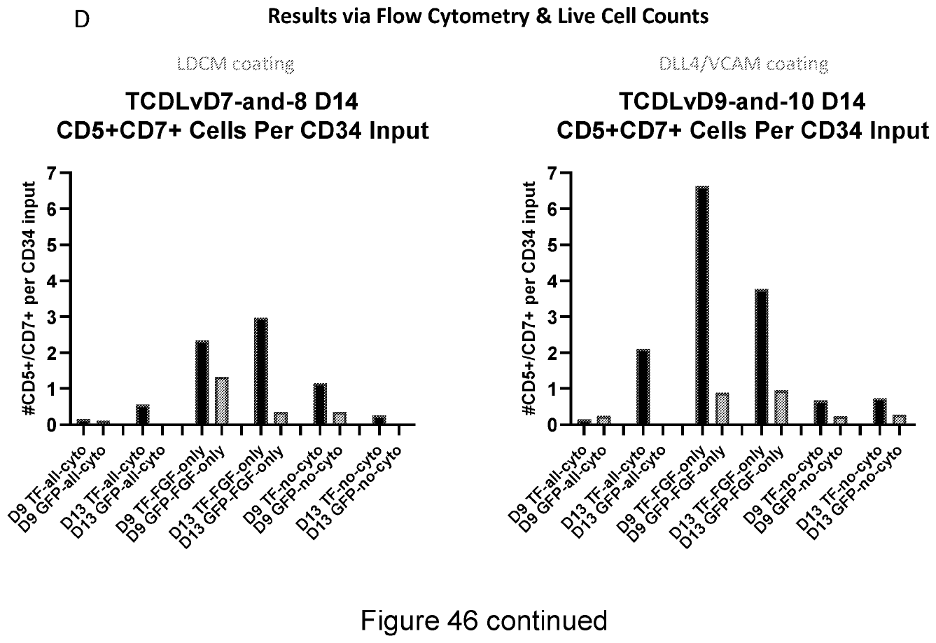

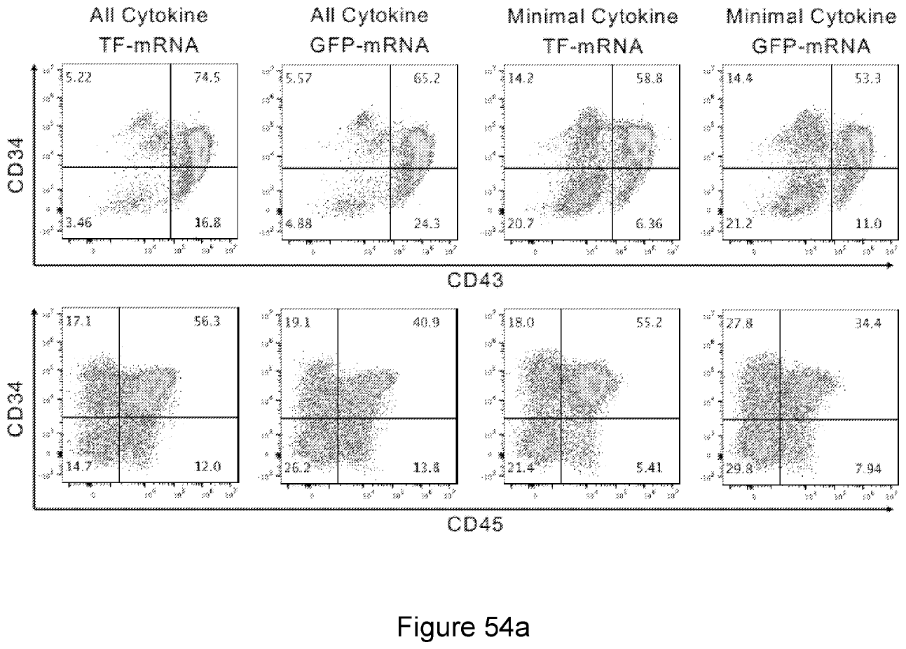

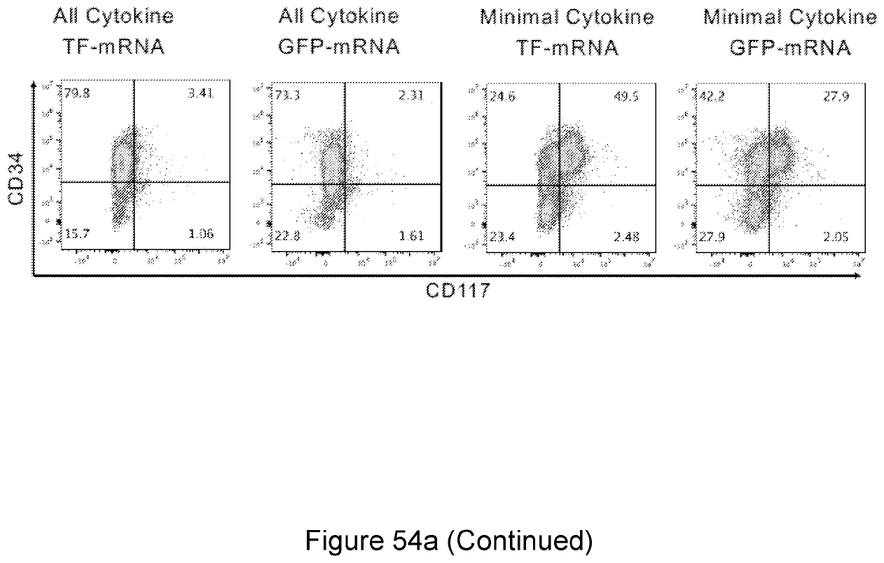

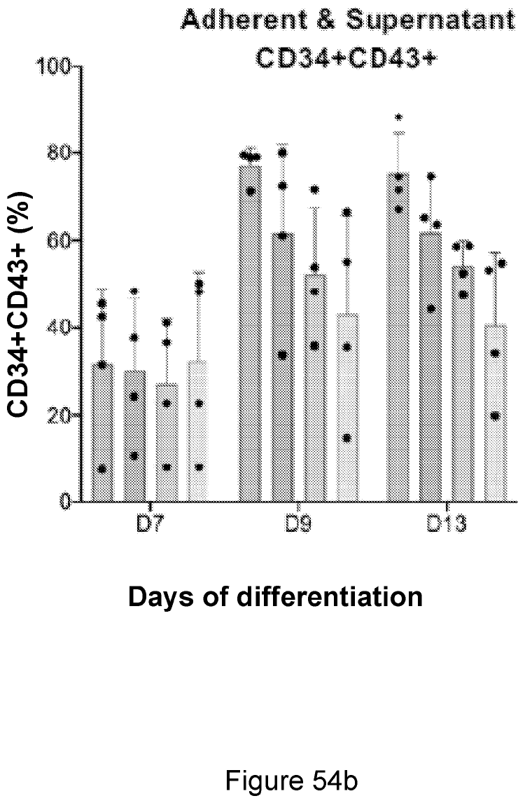

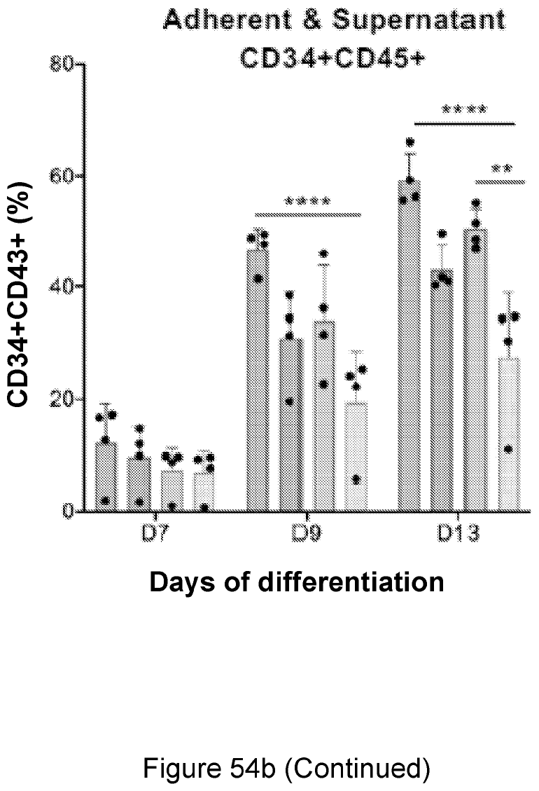

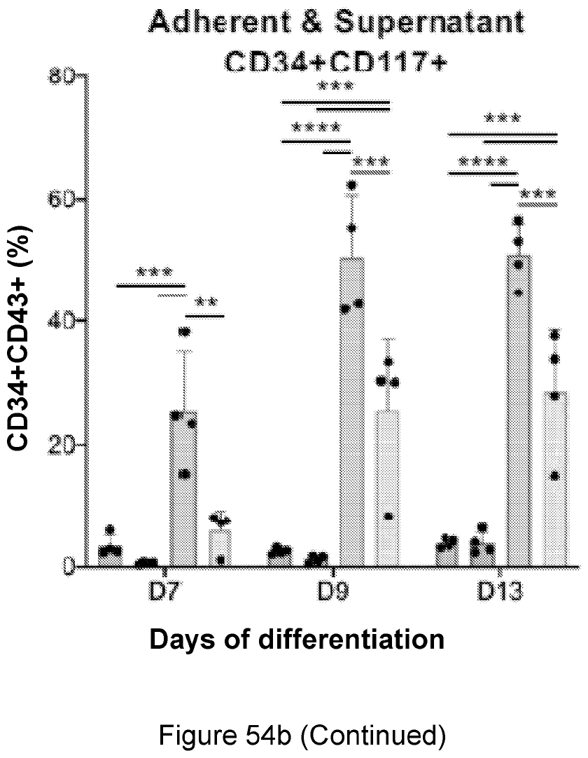

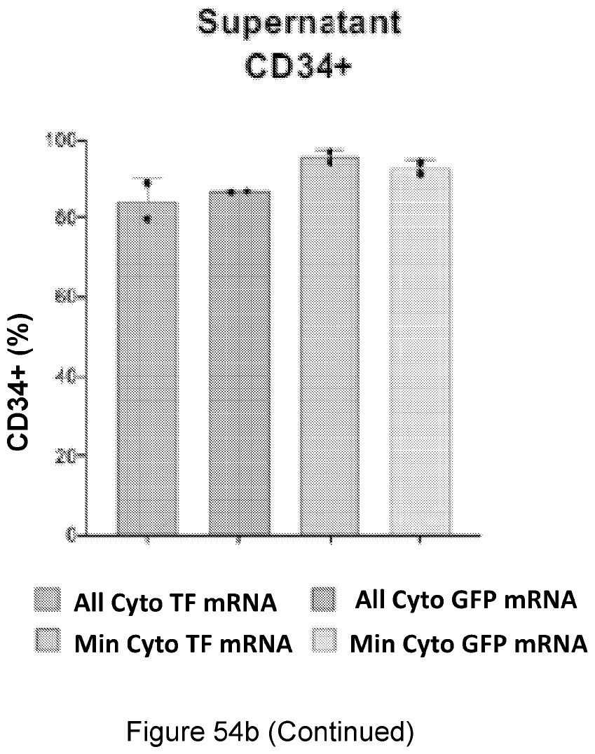

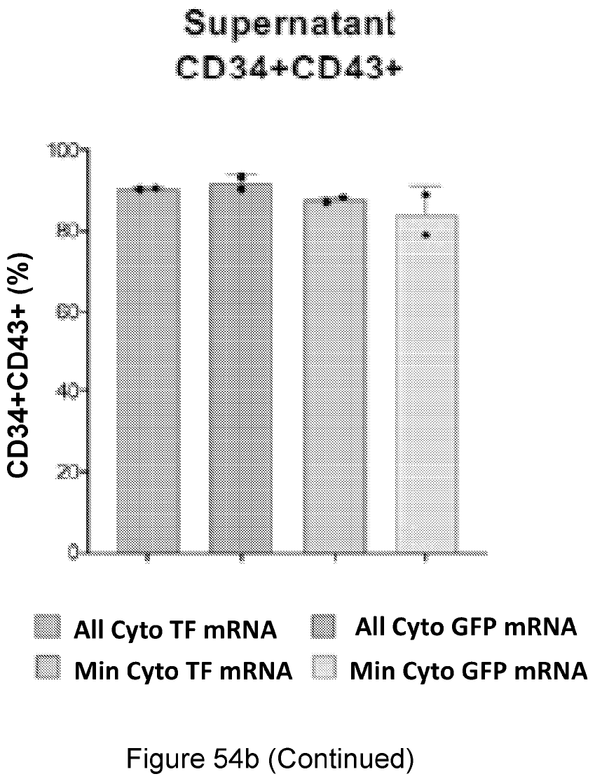

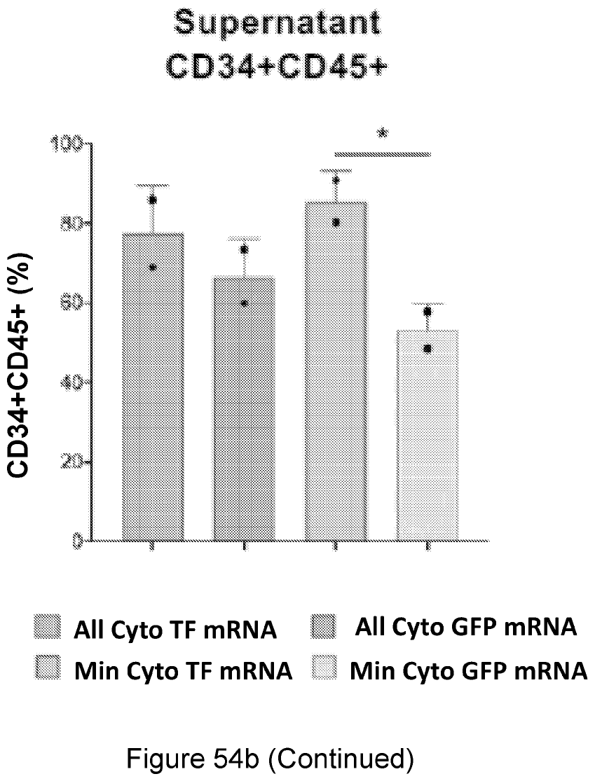

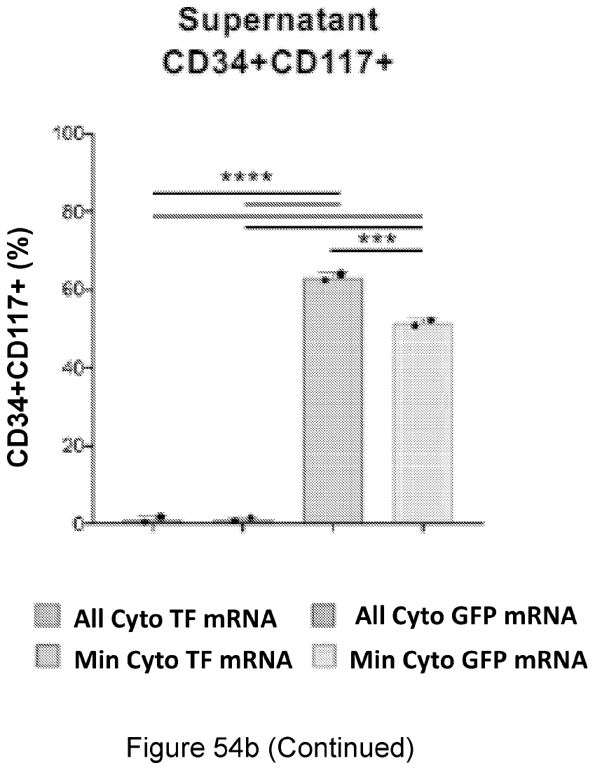

Unexpectedly, the inventors discovered that though the CD34⁺ HSPCs are substantially similar to naturally occurring HSPCs, the HSPCs generated by the disclosed methods have substantially higher C-kit (CD117) expression than naturally occurring and can be differentiated from CD34⁺ HSPC on at least this basis (FIG. 45, FIG. 54b).

The inventors discovered that the HSPCs generated by the disclosed methods can be efficiently differentiated into T cells or myeloid cells by culturing in the presence of DLL4 and optionally VCAM.

Accordingly, in some embodiments, the disclosed methods further comprise culturing the CD34⁺ HSPCs in the presence of one or more T cell differentiation factors selected from delta like canonical notch ligand 4 (DLL4) and vascular cell adhesion molecule (VCAM) in an amount effective to generate T cells.

Generation of in vitro differentiated T cells from induced pluripotent stem cells (iPSCs)

As described above, the inventors discovered that the HSPCs generated from iPSCs using the disclosed methods could efficiently be differentiated into T cells.

Accordingly, in another aspect of the current disclosure, methods of generating in vitro differentiated T cells from induced pluripotent stem cells are provided. In some embodiments, the methods comprise: (i) contacting the iPSCs with one or more mesoderm differentiation factors selected from bone morphogenetic protein 4 (BMP4), activin A, and CHIR99021; (ii) contacting the cells of step (i) with one or more HSPC differentiation factors selected from basic fibroblast growth factor (bFGF), vascular endothelial growth factor (VEGF), stem cell factor (SCF), and SB431542; (iii) contacting the cells of step (ii) with a plurality of nucleic acids encoding one or more transcription factors selected from ETS-related gene (ERG), ligand-dependent corepressor (LCOR), homeobox A5 (HOXA5), homeobox A9 (HOXA9), and runx family transcription factor 1 (RUNX1); (iv) contacting the cells of step (iii) with one or more HSPC differentiation factors selected from bFGF, VEGF, SCF, SB431542, and Y-27632; (v) culturing the cells of step (iv) in the presence of one or more T cell differentiation factors selected from delta like canonical notch ligand 4 (DLL4) and vascular cell adhesion molecule (VCAM) to generate T cells.

The T cells derived from the present disclosure differentially express CD16 and NKG2D as compared to in vivo developed T cells. Expression of such marker may allow for additional innate cytotoxic properties.

The inventors discovered that, in the case of generating T cells in vitro from iPSCs, iPSCs that were derived from T cells isolated from subjects more efficiently generated T cells in the disclosed methods. Without wishing to be limited by any theory or mechanism, the inventors believe that iPSCs derived from T cells from a subject have a somatically recombined T cell receptor coding sequence present in their genetic code, allowing them to express a functional T cell receptor once they have been differentiated into T cells. In contrast, iPSCs that are derived from, e.g., fibroblasts, do not have a somatically recombined T cell receptor coding sequence present in their genome and, thus, will not express a functional T cell receptor when differentiated in vitro into T cells.

Accordingly, in some embodiments, the iPSCs of step (i) of the above method of generating T cells in vitro are derived from T cells from a subject.

The inventors discovered that the disclosed methods of generating HSPCs and T cells in vitro can be performed with defined media, i.e., media with all defined components, and without any added serum. Therefore, the inventors envision that the disclosed methods are highly translatable to clinical applications, as they do not require the use of any antigenic or xenogeneic reagents.

### Cells

In another aspect of the current disclosure, cells generated by the disclosed methods are provided.

In some embodiments, CD34⁺ hematopoietic stem progenitor cells (HSPCs) generated from induced pluripotent stem cells (iPSCs) in vitro are provided. In some embodiments, the methods of generating CD34⁺ cells comprise: (i) contacting iPSCs with one or more mesoderm differentiation factors selected from bone morphogenetic protein 4 (BMP4), activin A, and CHIR99021; (ii) subsequently contacting the cells of step (ii) with one or more HSPC differentiation factors selected from basic fibroblast growth factor (bFGF), vascular endothelial growth factor (VEGF), stem cell factor (SCF), and SB431542; (iii) contacting the cells of step (iii) with a plurality of nucleic acids encoding one or more transcription factors selected from ETS-related gene (ERG), ligand-dependent corepressor (LCOR), homeobox A5 (HOXA5), homeobox A9 (HOXA9), and runx family transcription factor 1 (RUNX1); (iv) contacting the cells of step (iii) with one or more HSPC differentiation factors selected from bFGF, VEGF, SCF, SB431542, and Y-27632 to generate CD34⁺HSPCs.

In some embodiments, the CD34⁺ HSCPs are further genetically modified such that they comprise 1 or more exogenous polynucleotide. As used herein, “exogenous polynucleotide” is a polynucleotide that is introduced into a cell. In some embodiments, the exogenous polynucleotide may encode a naturally occurring or non-naturally occurring genetically engineered polynucleotide operably linked to one or more promoter for expression in the cell.

In some embodiments, in vitro generated T cells are provided. In some embodiments the in vitro generated T cells are generated by the method comprising: (i) contacting the cells with one or more mesoderm differentiation factors selected from bone morphogenetic protein 4 (BMP4), activin A, and CHIR99021; (ii) contacting the cells of step (i) with one or more HSPC differentiation factors selected from basic fibroblast growth factor (bFGF), vascular endothelial growth factor (VEGF), stem cell factor (SCF), and SB431542; (iii) contacting the cells of step (ii) with a plurality of nucleic acids encoding one or more transcription factors selected from ETS-related gene (ERG), ligand-dependent corepressor (LCOR), homeobox A5 (HOXA5), homeobox A9 (HOXA9), and runx family transcription factor 1 (RUNX1); (iv) contacting the cells of step (iii) with one or more HSPC differentiation factors selected from bFGF, VEGF, SCF, SB431542, and Y-27632; (v) culturing the cells of step (iv) in the presence of one or more T cell differentiation factors selected from delta like canonical notch ligand 4 (DLL4) and vascular cell adhesion molecule (VCAM) to generate T cells.

In some embodiments, the in vitro generated T cells are further genetically modified such that they comprise one or more exogenous polynucleotide. In some embodiments, the exogenous polynucleotide may encode a naturally occurring or a non-naturally occurring genetically engineered polynucleotide operably linked to one or more promoter for expression in the cell.

In some embodiments, in vitro generated myeloid cells are provided, including granulocytes, erythrocytes, monocytes, megakaryocytes, and/or macrophages. In some embodiments the in vitro generated myeloid cells are generated by the method comprising: (i) contacting the iPSCs with one or more mesoderm differentiation factors selected from bone morphogenetic protein 4 (BMP4), activin A, and CHIR99021; (ii) contacting the cells of step (i) with one or more HSPC differentiation factors selected from basic fibroblast growth factor (bFGF), vascular endothelial growth factor (VEGF), stem cell factor (SCF), and SB431542; (iii) contacting the cells of step (ii) with a plurality of nucleic acids encoding one or more transcription factors selected from ETS-related gene (ERG), ligand-dependent corepressor (LCOR), homeobox A5 (HOXA5), homeobox A9 (HOXA9), and runx family transcription factor 1 (RUNX1); (iv) contacting the cells of step (iii) with one or more HSPC differentiation factors selected from bFGF, VEGF, SCF, SB431542, and Y-27632; (v) culturing the cells of step (iv) in the presence of one or more myeloid differentiation factors selected from delta like canonical notch ligand 4 (DLL4) and vascular cell adhesion molecule (VCAM) to generate T cells.

The present invention is described herein using several definitions, as set forth below and throughout the application.

### Pharmaceutical Compositions

In another aspect of the current disclosure, pharmaceutical compositions are provided.

In some embodiments, pharmaceutical compositions comprising CD34⁺ hematopoietic stem progenitor cells (HSPCs) generated from induced pluripotent stem cells (iPSCs) in vitro are provided. In some embodiments, the pharmaceutical composition comprises cells that are generated by the method comprising: (i) contacting iPSCs with one or more mesoderm differentiation factors selected from bone morphogenetic protein 4 (BMP4), activin A, and CHIR99021; (ii) subsequently contacting the cells of step (i) with one or more HSPC differentiation factors selected from basic fibroblast growth factor (bFGF), vascular endothelial growth factor (VEGF), stem cell factor (SCF), and SB431542; (iii) contacting the cells of step (ii) with a plurality of nucleic acids encoding one or more transcription factors selected from ETS-related gene (ERG), ligand-dependent corepressor (LCOR), homeobox A5(HOXA5), homeobox A9 (HOXA9), and runx family transcription factor 1 (RUNX1); (iv) contacting the cells of step (iii) with one or more HSPC differentiation factors selected from bFGF, VEGF, SCF, SB431542, and Y-27632 to generate CD34+HSPCs; and a pharmaceutically acceptable carrier.

In some embodiments, pharmaceutical compositions comprising in vitro generated T cells are provided. In some embodiments the pharmaceutical compositions comprise T cells that are generated by the method comprising: (i) contacting iPSCs with one or more mesoderm differentiation factors selected from bone morphogenetic protein 4 (BMP4), activin A, and CHIR99021; (ii) contacting the cells of step (i) with one or more HSPC differentiation factors selected from basic fibroblast growth factor (bFGF), vascular endothelial growth factor (VEGF), stem cell factor (SCF), and SB431542; (iii) contacting the cells of step (ii) with a plurality of nucleic acids encoding one or more transcription factors selected from ETS-related gene (ERG), ligand-dependent corepressor (LCOR), homeobox A5 (HOXA5), homeobox A9(HOXA9), and runx family transcription factor 1 (RUNX1); (iv) contacting the cells of step (iii) with one or more HSPC differentiation factors selected from bFGF, VEGF, SCF, SB431542, and Y-27632; (v) culturing the cells of step (iv) in the presence of one or more T cell differentiation factors selected from delta like canonical notch ligand 4 (DLL4) and vascular cell adhesion molecule (VCAM) to generate T cells; and a pharmaceutically acceptable carrier.

### Methods of Treatment

In another aspect of the current disclosure, methods of treating a subject in need of reconstitution of hematopoietic stem progenitor cells (HSPCs) are provided. In some embodiments, the methods comprise administering an effective amount of a pharmaceutical composition comprising cells that are generated by the method comprising: (i) contacting iPSCs with one or more mesoderm differentiation factors selected from bone morphogenetic protein 4 (BMP4), activin A, and CHIR99021; (ii) subsequently contacting the cells of step (i) with one or more HSPC differentiation factors selected from basic fibroblast growth factor (bFGF), vascular endothelial growth factor (VEGF), stem cell factor (SCF), and SB431542; (iii) contacting the cells of step (ii) with a plurality of nucleic acids encoding one or more transcription factors selected from ETS-related gene (ERG), ligand-dependent corepressor (LCOR), homeobox A5 (HOXA5), homeobox A9 (HOXA9), and runx family transcription factor 1 (RUNX1); (iv) contacting the cells of step (iii) with one or more HSPC differentiation factors selected from bFGF, VEGF, SCF, SB431542, and Y-27632 to generate CD34⁺ HSPCs; and a pharmaceutically acceptable carrier to a subject in need thereof to reconstitute CD34⁺ HSPCs in the subject.

As used herein, “a subject in need of reconstitution of hematopoietic stem progenitor cells (HSPCs)” refers to a subject who has reduced or pathological hematopoiesis, e.g., a subject for which a bone marrow transplant is indicated. In some embodiments, the subject has a blood or bone marrow disorder or disease such as leukemia or lymphoma or exhibits altered or pathological hematopoiesis. In some embodiments, a subject has been diagnosed with a bone marrow proliferative disease or disorder, e.g., polycythemia vera. In some embodiments, a subject has been diagnosed with a bone marrow failure syndrome, e.g., Fanconi anemia. Thus, in some embodiments, the aforementioned subjects may benefit from bone marrow reconstituted with CD34⁺ HSPCs.

In some embodiments, the subject must undergo bone marrow ablation prior to transfer of CD34⁺ cells, a process for which methods are known and understood in the art.

The methods of reconstituting CD34⁺ HSCPs in a subject are contemplated to be performed as an autologous transplant. In other words, the methods of generating CD34⁺ HSPCs are performed using iPSCs derived from the subject who is the intended recipient of the transplant. However, in some embodiments, a subject in need thereof may be suffering from a disease or disorder that is inherited and, thus, may not be a candidate for autologous CD34⁺ iPSC derived HSPC reconstitution, e.g., in the case of inherited bone marrow failure disorders. However, CD34⁺ HSPCs could be derived from iPSCs from a closely related individual that is unaffected by the disease or disorder precluding the use of an autologous CD34⁺ iPSC derived HSPC reconstitution, e.g., an unaffected sibling, parent, half-sibling, etc.

Accordingly, successful reconstitution of a subject with CD34⁺ HSPCs derived from iPSCs may be characterized by normal hematopoiesis in the subject, i.e., production of the full range of hematopoietically-derived cells, e.g., lymphocytes, monocytes, granulocytes, erythrocytes, megakaryocytes/platelets, etc. at about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months after reconstitution by the disclosed methods. In some embodiments, the subject does not experience signs or symptoms of graft-versus-host disease (GVHD) which may include, but are not limited to rash, abdominal pain, jaundice, diarrhea, nausea, ulcers in the mouth, dry eyes, frequent infections, unexplained weight loss/failure to thrive at about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months after reconstitution by the disclosed methods.

In another aspect of the current disclosure, methods of treating a subject in need of T cell adoptive transfer are provided. In some embodiments, the methods comprise: administering an effective amount of a pharmaceutical composition comprising T cells that are generated by the method comprising: (i) contacting iPSCs with one or more mesoderm differentiation factors selected from bone morphogenetic protein 4 (BMP4), activin A, and CHIR99021; (ii) contacting the cells of step (i) with one or more HSPC differentiation factors selected from basic fibroblast growth factor (bFGF), vascular endothelial growth factor (VEGF), stem cell factor (SCF), and SB431542; (iii) contacting the cells of step (ii) with a plurality of nucleic acids encoding one or more transcription factors selected from ETS-related gene (ERG), ligand-dependent corepressor (LCOR), homeobox A5 (HOXA5), homeobox A9 (HOXA9), and runx family transcription factor 1 (RUNX1); (iv) contacting the cells of step (iii) with one or more HSPC differentiation factors selected from bFGF, VEGF, SCF, SB431542, and Y-27632; (v) culturing the cells of step (iv) in the presence of one or more T cell differentiation factors selected from delta like canonical notch ligand 4 (DLL4) and vascular cell adhesion molecule (VCAM) to generate T cells; and a pharmaceutically acceptable carrier to the subject as a T cell adoptive transfer.

As used herein, “a subject in need of T cell adoptive transfer” refers to a subject that would benefit from adoptively transferred T cells. T cell adoptive transfer, also referred to as “adoptive cell therapy (ACT)” refers to transferring T cells to a subject, typically intravenously. Adoptive cell transfer is used to introduce T cells that possess a certain immunological function, e.g., tumor infiltrating lymphocytes (TILs), chimeric antigen receptor T (CAR-T) cells, virus-specific T cells, e.g., HIV-specific T cells into a subject, whereafter, the adoptively transferred T cells destroy transformed or virally infected cells. Therefore, in some embodiments, a subject in need of T cell adoptive transfer has been diagnosed with cancer or a chronic viral infection. The methods of treating a subject in need of T cell adoptive transfer are contemplated to be performed as an autologous transfer. In other words, the methods of generating in vitro generated T cells are performed using iPSCs derived from the subject who is the intended recipient of the adoptive T cell transfer. In the case of adoptive T cell transfer, autologous transfers are advantageous for a variety of reasons including, but not limited to, major and minor human leukocyte antigen (HLA) matching and prevention of acute and chronic GVHD resulting from such mismatches.

As discussed above, the inventors discovered that iPSCs that were derived from T cells isolated from subjects more efficiently generated T cells from said iPSCs in the disclosed methods of generating T cells in vitro, than T cells derived from iPSCs generated from fibroblasts. Accordingly, in some embodiments, the methods of treatment are performed using in vitro generated T cells that are derived from iPSCs generated from T cells isolated from a subject. In some embodiments, the subject from which the T cells were isolated and used to generate iPSCs, and ultimately in vitro generated T cells, is the subject who is the intended recipient of the T cell adoptive transfer, i.e., an autologous transfer.

In some embodiments, the in vitro generated T cells are further genetically modified such that they comprise one or more exogenous polynucleotide. In some embodiments, the one or more exogenous polynucleotides encode a chimeric antigen receptor, expression of which is operably linked to a promoter.

Accordingly, treatment of a subject with T cell adoptive transfer according to the disclosed methods to treat a tumor may comprise reduction in tumor volume or tumor burden, reduction in tumor prevalence, for example in the case of blood cancers, increased time to progression, remission, etc. Treatment of a subject with T cell adoptive transfer according to the disclosed methods to treat a chronic viral infection may comprise reduced symptoms of the particular viral infection, e.g., HIV infection-increased CD4 T cell count, reduced viral load, etc.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The term “subject” may be used interchangeably with the terms “individual” and “patient” and includes human and non-human mammalian subjects.

The phrase “pharmaceutically acceptable” means that which is useful in preparing a pharmaceutical composition that is generally safe, non-toxic and neither biologically nor otherwise undesirable and includes that which is acceptable for use in a subject, including human pharmaceutical use.

The phrase “pharmaceutically acceptable carrier,” as known in the art, refers to a pharmaceutically acceptable material, medium, composition, or vehicle, suitable for administering to a subject. Suitable carriers may include, for example, liquids (both aqueous and non-aqueous alike, and combinations thereof), solids, encapsulating materials, gases, and combinations thereof (e.g., semi-solids), that function to carry, transport, or deliver a material, compound, composition or the like from one cell, tissue, or organ, or portion of the body, to another cell, tissue, or organ, or portion of the body.

### EXAMPLES

The following Examples are illustrative and should not be interpreted to limit the scope of the claimed subject matter.

### Example 1—Generation of HSPCs and T Cells From Ipscs Using Transient Delivery of mRNA Encoding Transcription Factors

A commercially available 1210 iPSC line derived from human dermal fibroblast was used to differentiate into hematopoietic progenitor cells that express CD34. Three methods were evaluated for over-expression of hematopoietic transcription factors in iPSC cells that were undergoing differentiation into hematopoietic progenitor cells.

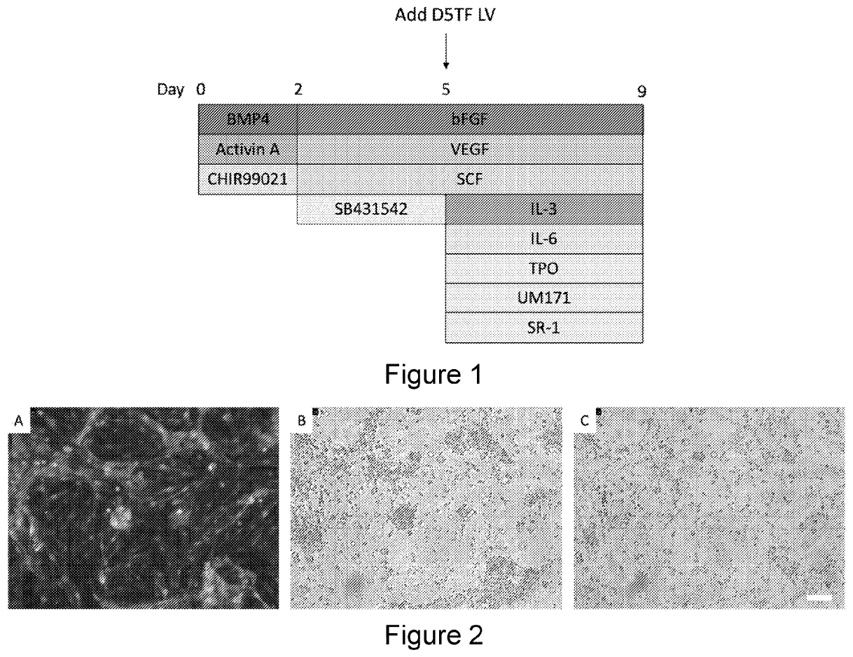

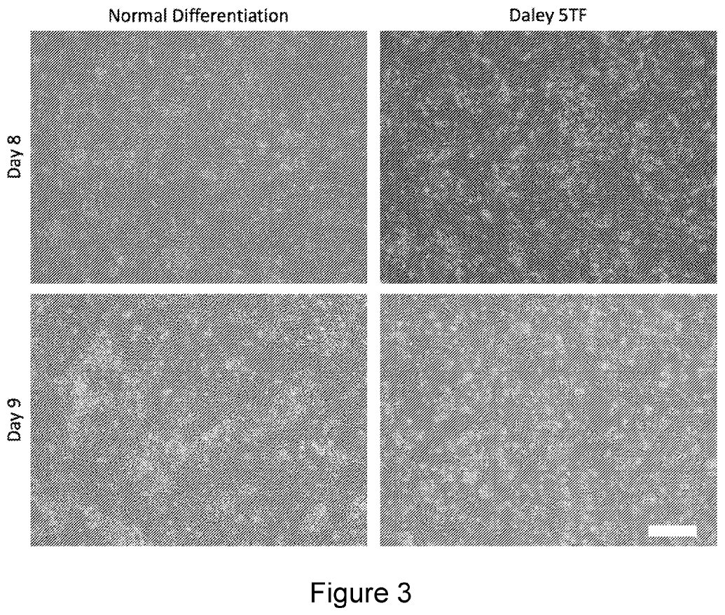

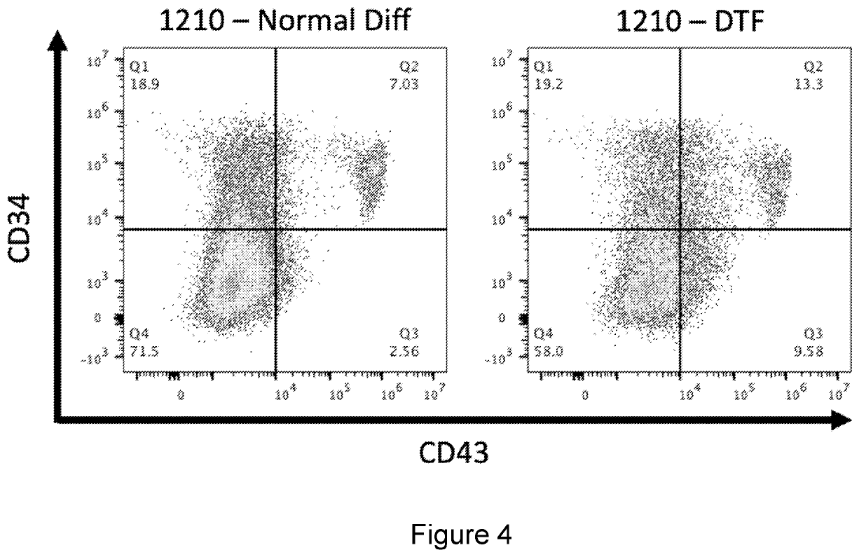

Initial experiments utilized a lentivirus encoding five hematopoietic transcription factors LCOR, HOXA9, HOXA5, RUNX1 and ERG, along with a GFP reporter signal. The lentivirus, referred to as D5TF, was added on Day 5 of the iPSC-CD34 differentiation protocol (FIG. 1) using a MOI of 25 (~MOI 5 for each TF). On Day 7, two days after transduction, cells were imaged and all expressed GFP (FIG. 2). While GFP expression was high, cell viability decreased with extended culture. On Day 9, cells that did not receive the lentivirus were 77% viable compared to 44% for those that received the lentivirus on Day 5 (FIG. 3). To determine the frequency of hematopoietic progenitor cells, we looked for CD34 and CD43 positive cells. Although there was a slight increase in CD34+CD43+ cells that received the D5TF lentivirus, overall the expression profile was similar to control conditions (FIG. 4). Due to the toxicity of the D5TF lentivirus and small difference in CD34+CD43+ expression between controls we discontinued using the D5TF lentivirus.

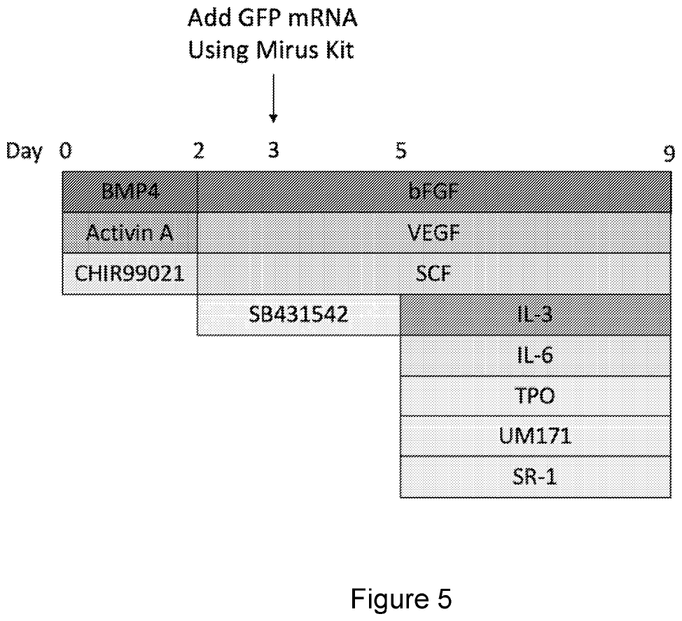

The second method used to incorporate mRNA involved a Mirus TransIT®-mRNA Transfection Kit. For this experiment GFP mRNA was added with the Mirus kit on Day 3 of the iPSC-CD34 differentiation protocol (FIG. 5). Two different concentrations of the Mirus kit were used (2.5 μl and 5 μL) and compared to a control condition that did not receive any mRNA during the iPSC-CD34 differentiation.

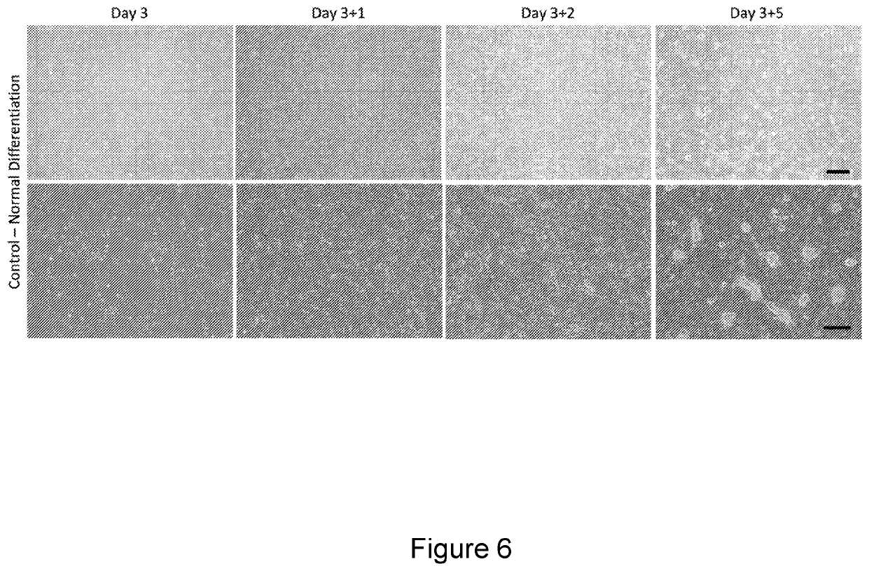

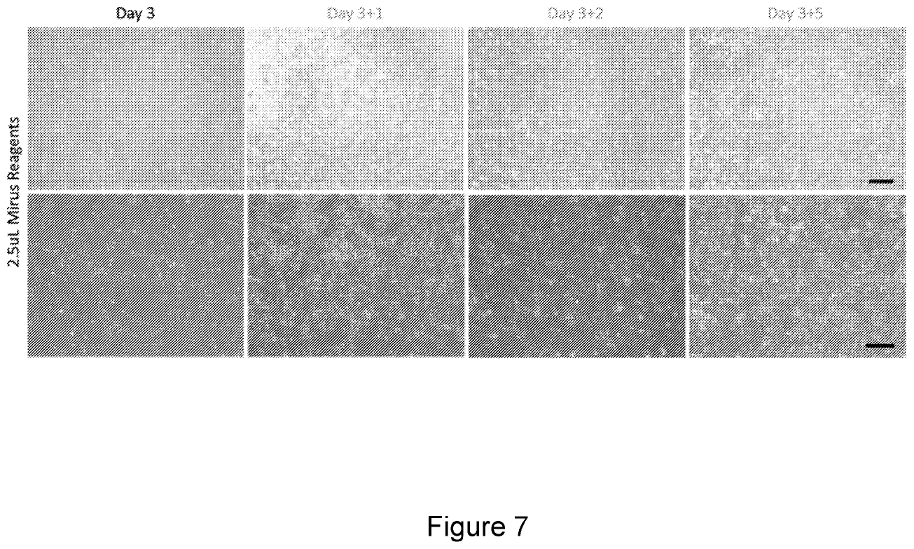

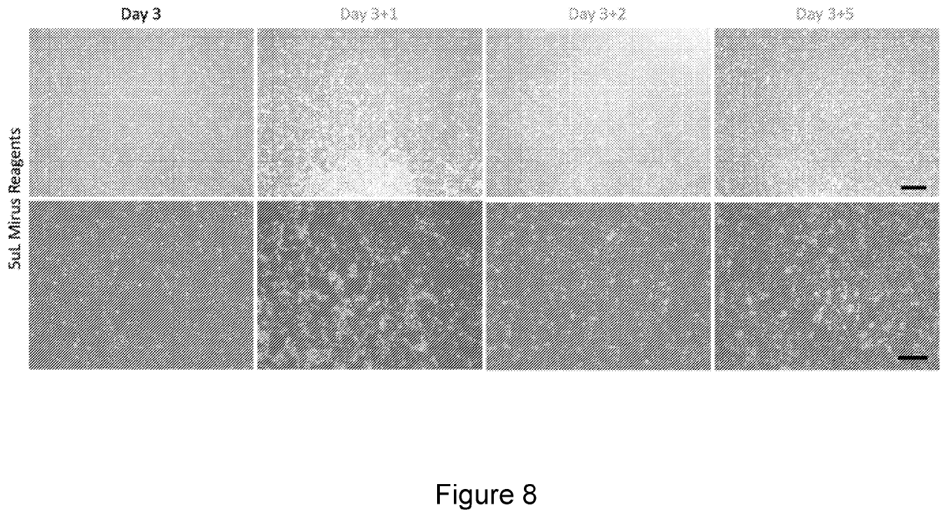

Bright field images were taken of the three conditions during the differentiation (FIGS. 6-8). Compared to the control condition, both the 2.5 and 5 μL Mirus conditions were toxic to the cells as indicated by cell death and a lack of a monolayer of cells on Day 3+1. The 5 μL Mirus condition was more toxic than the 2.5 μL Mirus condition and took longer for the cells to recover and form a complete monolayer.

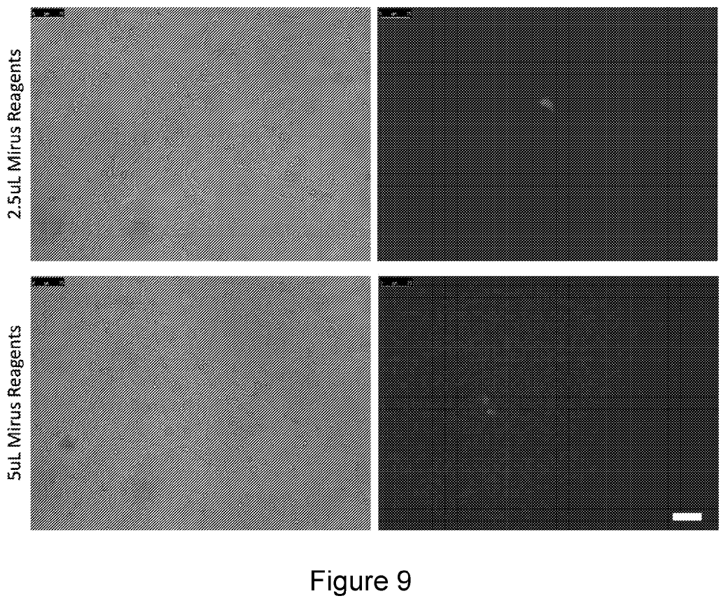

Unlike the D5TF lentivirus condition in which all cells expressed GFP, the Mirus kit had little GFP+cells irrespective of the Mirus concentration used (FIG. 9). Due to the toxicity and low levels of GFP+cells, the Mirus kit was discontinued as a way to introduce mRNA into iPSC cells undergoing differentiation into hematopoietic progenitors.

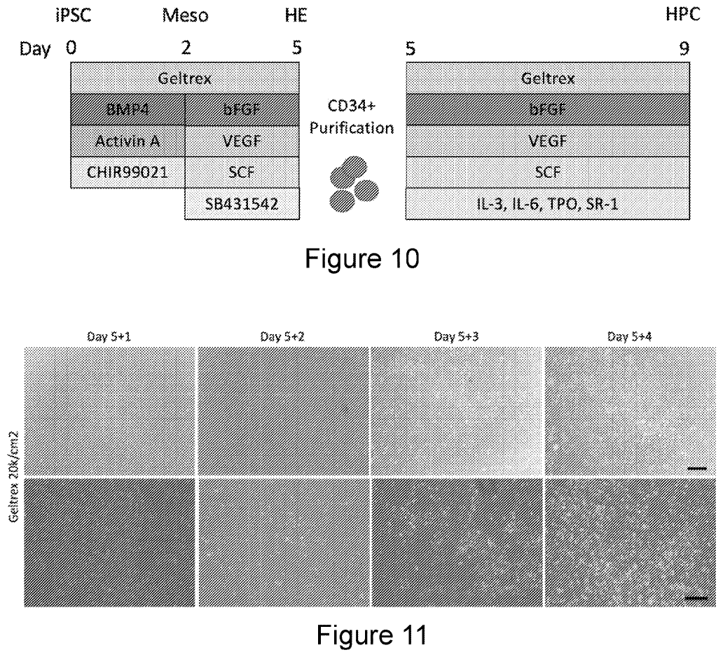

The last method used to incorporate mRNA was the Neon electroporation system. Prior to transfecting the cells with mRNA, we first determined which cells would receive mRNA. The first option was to magnetically purify the CD34+ cells on Day 5 and then transfect with mRNA using the Neon electroporation system (FIG. 10). Prior to transfection, we first determined how many purified CD34+ cells would be needed to be re-seeded to form a complete monolayer by Day 9.

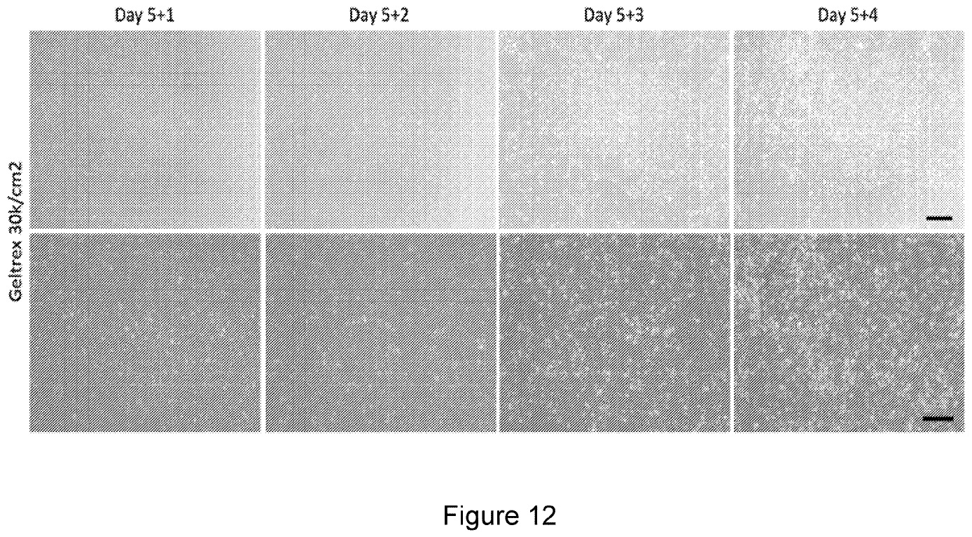

On Day 5, CD34+ cells were magnetically selected and re-seeded into new wells coated with Geltrex at two different densities, 20 k CD34+ cells/cm2 and 30 k CD34+ cells/cm2 (FIG. 11 and FIG. 12, respectively). As expected, the 30 k/cm2 condition had more cells than the 20 mk/cm2 one day after re-seed and formed a complete monolayer two days after re-seed. Unexpectedly, cells in both densities started to detach from the surface and die at the end of the differentiation protocol on Day 9.

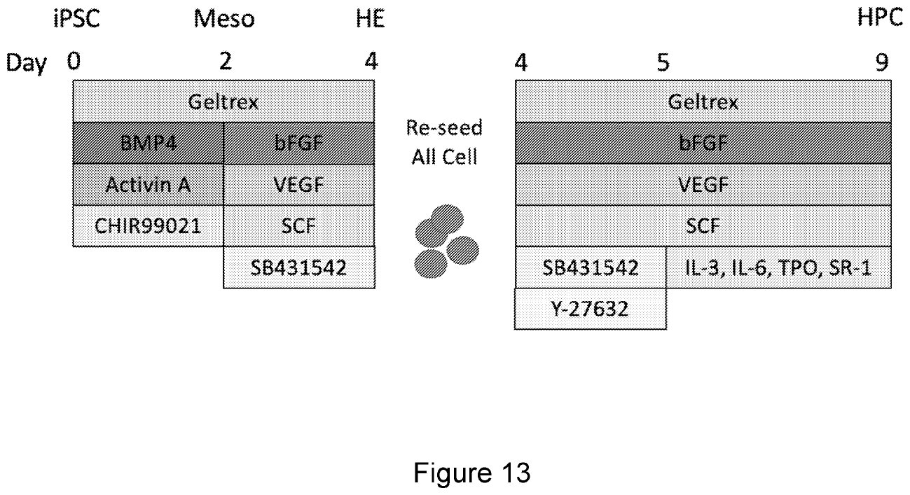

Next, we tried to re-seed all cells instead of magnetically purifying CD34+ cells (FIG. 13). In addition, we re-seeded all cells on Day 4 rather than Day 5 so that the cells would be in the same culture media as they were cultured from Days 2-4. On Day 5, a complete media change was performed to remove any unattached cells and new media was added that contained the Day 5-9 cytokines.

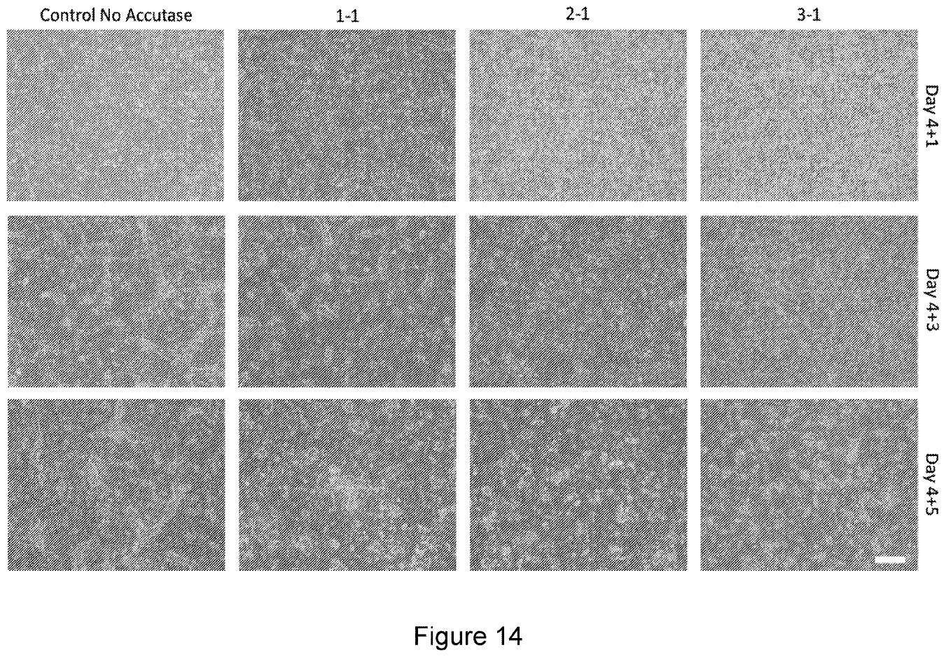

Three different re-seed densities were tried including 1 well being re-seeded into 1 new well (1-1), 2 wells collected together and re-seeded into 1 new well (2-1) and 3 wells collected together and re-seeded into 1 new well (3-1) (FIG. 14). All three re-seed conditions formed a complete monolayer two days after re-seed and maintained a monolayer until the end of the differentiation on Day 9. CD34 and CD43 expression was analyzed at three different time points after the cells were re-seeded (FIG. 15). One day after re-seed (Day 4+1) the three re-seed conditions had similar levels of CD34+ cells. Three days after re-seed (Day 4+3) the three re-seed conditions had a higher amount of CD34+CD43+ cells compared to the control no re-seed condition, with the highest amount of double positive cells occurring in the 3-1 re-seed condition. The same trend was observed for five days after re-seed (Day 4+5). These results demonstrate that re-seed alone on Day 4, without the addition of mRNA, can improve the CD34 output of differentiating iPSCs.

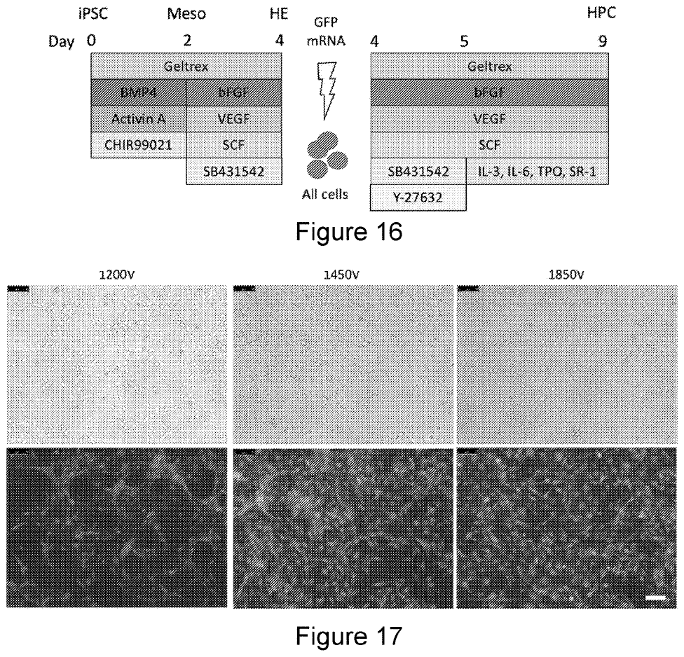

After identifying that all cells re-seed better than magnetically purified CD34+ cells, we next aimed to improve differentiation efficiency by incorporating mRNA into the re-seeded cells. The Neon electroporation parameters were first optimized with GFP mRNA (FIG. 16). The following three parameters were tested: 1) 1200V, 10 ms, Pulse number 3, 2) 1450V, 10 ms, Pulse number 3, 3) 1850V, 10 ms, Pulse number 3.

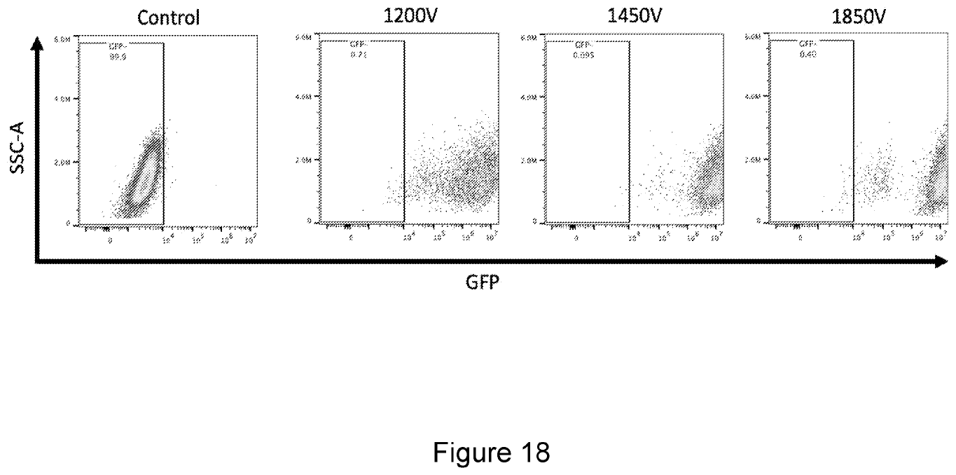

Both immunofluorescence (FIG. 17) and flow cytometry (FIG. 18) showed high levels of GFP expression 1 day post electroporation. Two days post electroporation high levels of viability were seen in the 1200V condition (93.5%) and 1450V condition (94%) while the 1800V condition had less viable cells (55.5%). In FIG. 18 the flow cytometry results show a small tail of less bright GFP+ cells whereas the 1450V condition has an even population of GFP+ cells; therefore, we moved forward with the 1450V condition.

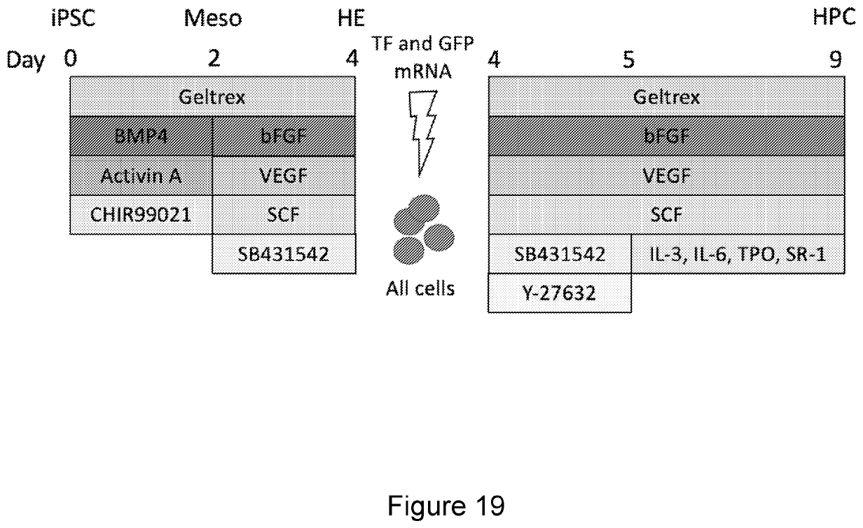

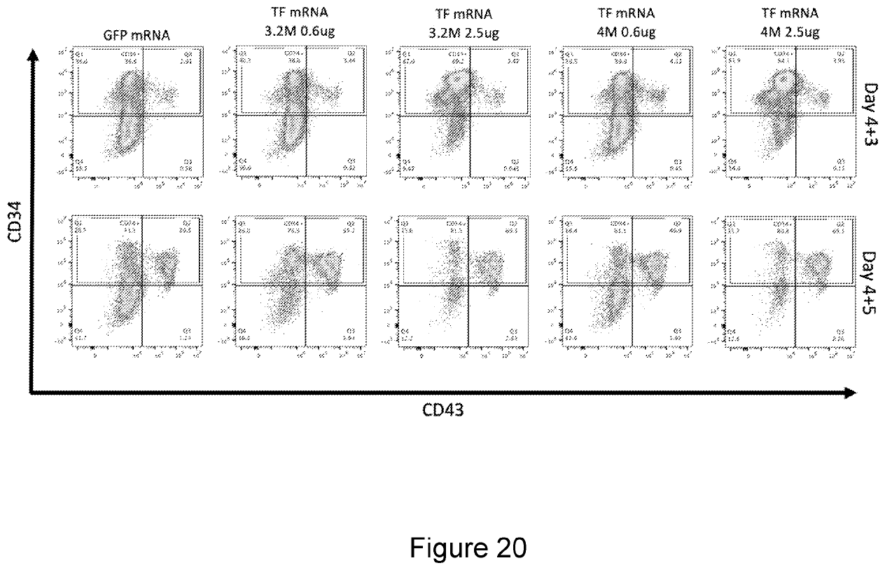

After optimizing the electroporation parameters with GFP mRNA, we next incorporated TF mRNA consisting of LCOR, HOXA9, HOXA5, RUNX1 and ERG, along with GFP mRNA used as a reporter signal (FIG. 19). Two different concentrations of TF mRNA were tested including 0.6 ug (0.12 ug/TF) and 2.5 ug (0.5 ug/TF). In addition, the total amount of cells electroporated was either 3.2M or 4M (FIG. 20). Three days post electroporation (Day 4+3) the 2.5 ug TF mRNA condition was more efficient at producing CD34+ cells compared to the 0.6 ug TF mRNA and GFP mRNA conditions. This trend remained for the Day 4+5 time point. The amount of cells electroporated did little to change the frequency of CD34+ cells. We moved forward with electroporating 4M cells on Day 4 with 2.5 ug of TF mRNA.

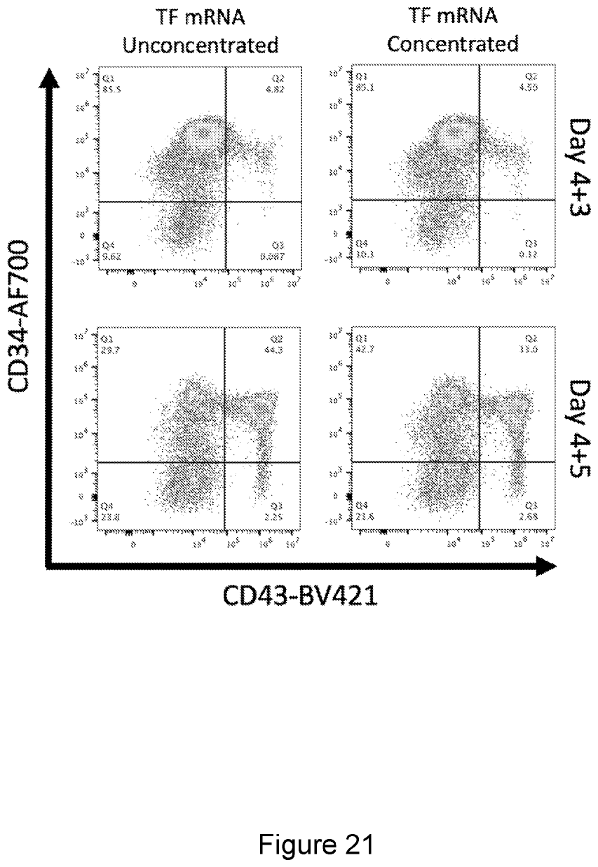

The last optimization performed was to concentrate the amount of TF mRNA delivered into a smaller volume. Using a Amicon® Ultra-15 Centrifugal Filter Units from EMD Millipore, we concentrated 2.5 ug of TF mRNA from 3.2 uL to 0.76 uL. As expected, the CD34+ cell frequency remained the same when using either concentrated or unconcentrated TF mRNA (FIG. 21). Although not necessary for the current work, the concentrated mRNA allows us to include more mRNA in future experiments (either more of the 5 TFs already used or include additional TFs) while still maintaining 70% of the T buffer electroporation solution. Prior work has shown that cell viability decreases post electroporation when the electroporation solution decreases below 70% (data not shown).

### Tf mRNA Generated Day 9 iPSC-HPCs

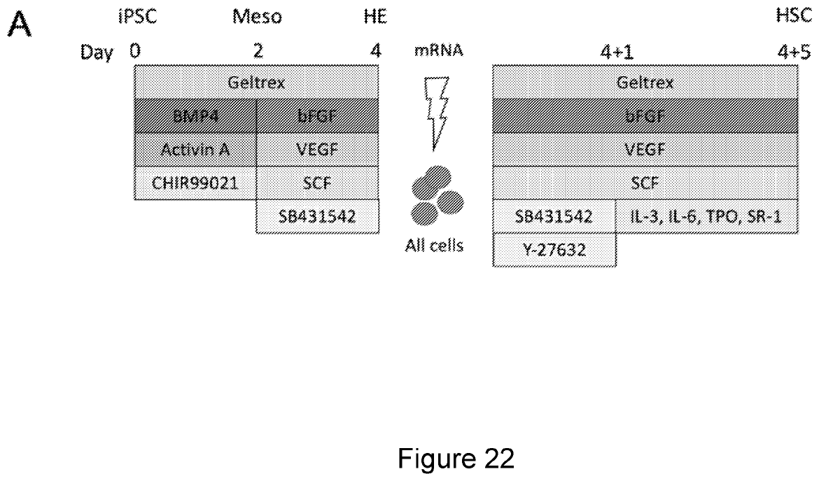

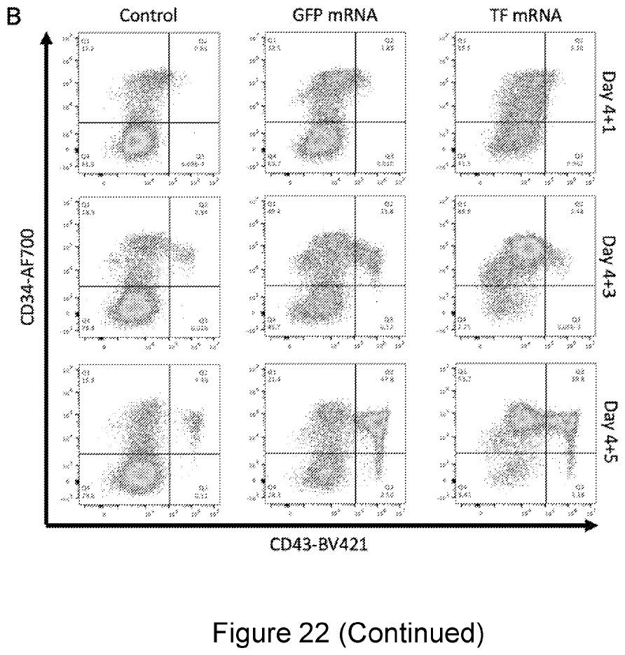

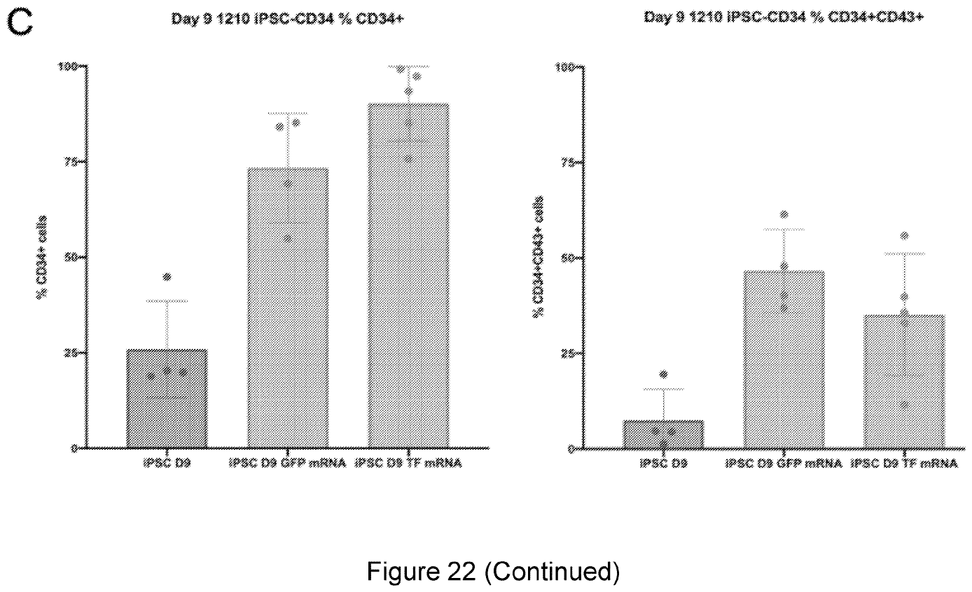

After optimizing the 2DMD protocol, we next sought to characterize the phenotype of the GFP and TF mRNA generated iPSC-HPCs. FIG. 22A shows the experimental outline, with cells either receiving GFP mRNA or TF mRNA on Day 4. As a Control we also included a condition that did not receive mRNA and was continuously cultured in the same well for 9 days (ie no accutase on Day 4). As seen in FIG. 22B the TF mRNA treated cells had 92% CD34+ cells 3 days post electroporation compared to 21% and 44% for the Control and GFP mRNA conditions. The same trend was observed on Day 4+5, notably only 7% of the cells were CD34− in the TF mRNA condition. This experiment was repeated five times and the results plotted in FIG. 22C.

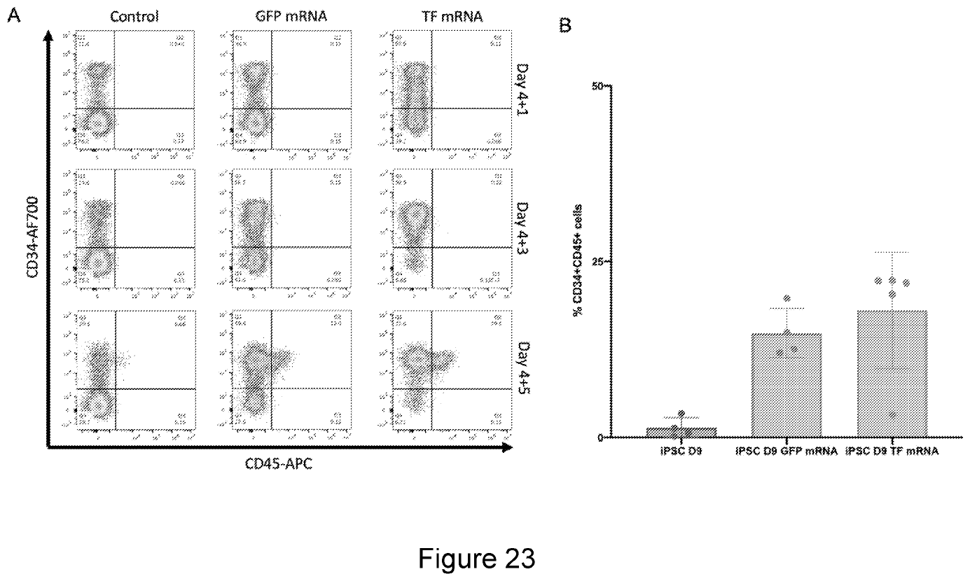

In addition to CD34 and CD43 expression, we also assessed the hematopoietic marker CD45 (FIG. 23). Both the GFP and TF mRNA conditions improved the frequency of CD34+CD45+ cells compared to the Control condition. This result remained consistent for multiple experiments (FIG. 23B).

### Day 9 iPSC-HPC Clonogenic Output

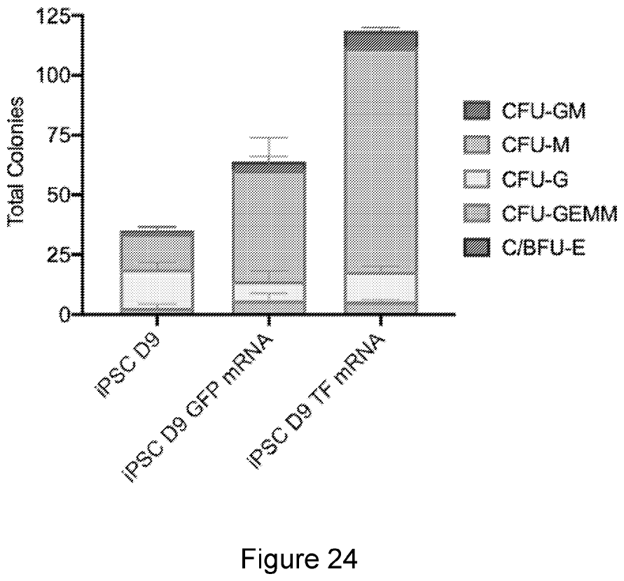

To assess the functionality of Day 9 iPSC-HPCs from the three conditions we performed a colony forming unit (CFU) assay (FIG. 24). As expected, both the GFP and TF mRNA treated cells generated more colonies than the Control iPSC condition. Although GFP mRNA cells had similar levels of CD34+CD43+(FIG. 22C) and CD34CD45+ (FIG. 23B) expression compared to TF mRNA treated cells, the total number of colonies produced by the GFP mRNA condition was significantly lower than the TF mRNA condition, with the largest difference observed in the CFU-M fraction.

### T Cells Generated From Day 9 iPSC-HPCs

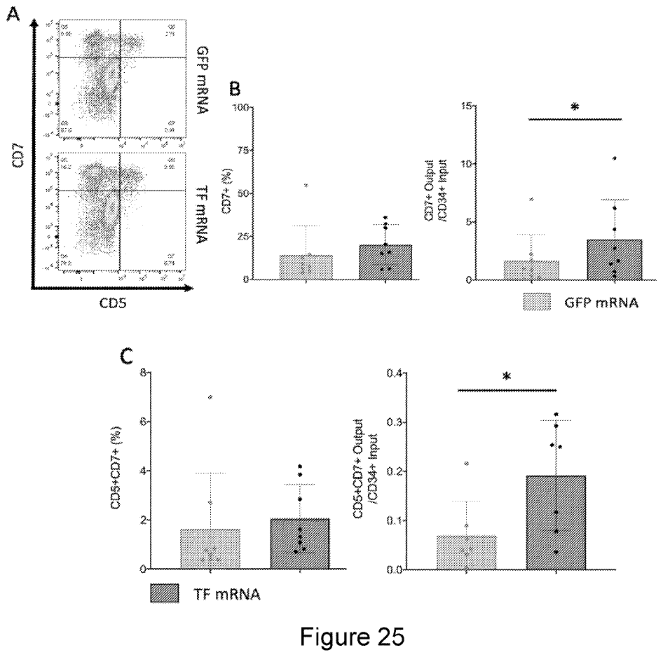

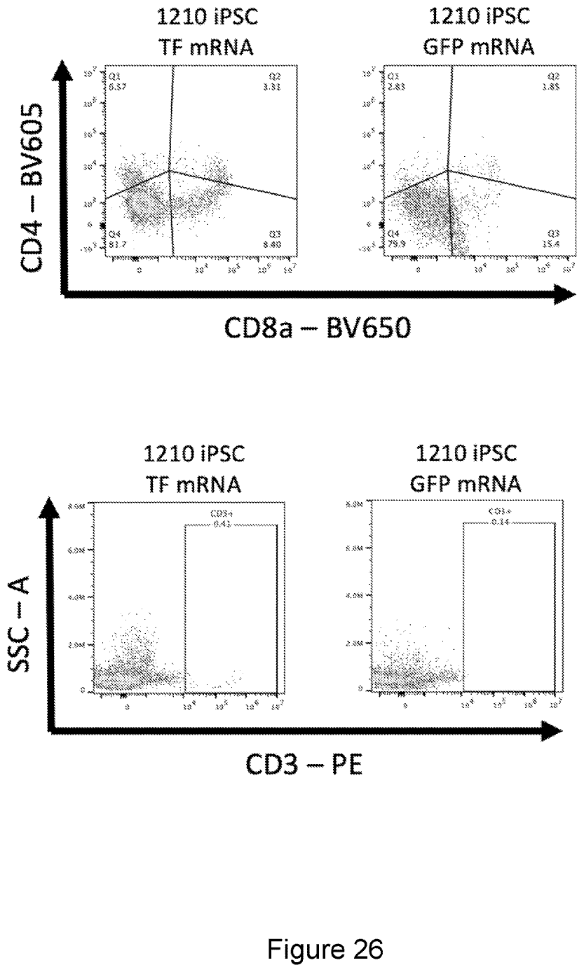

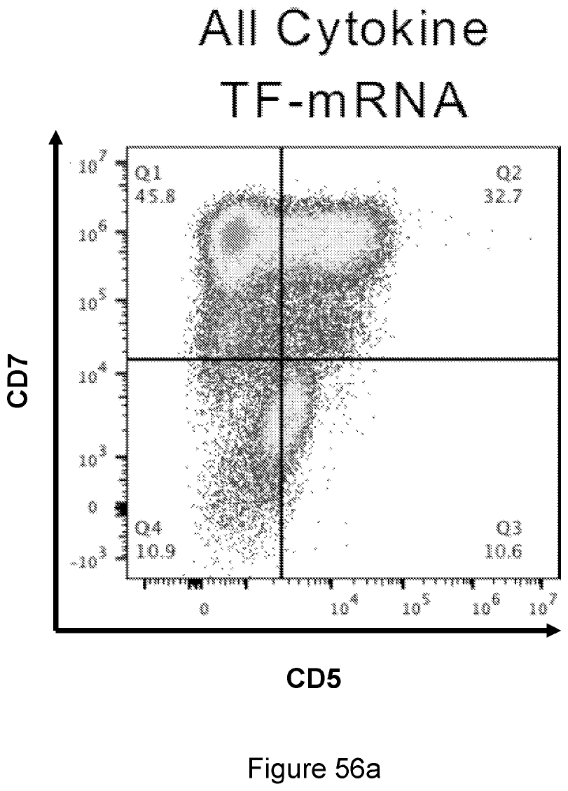

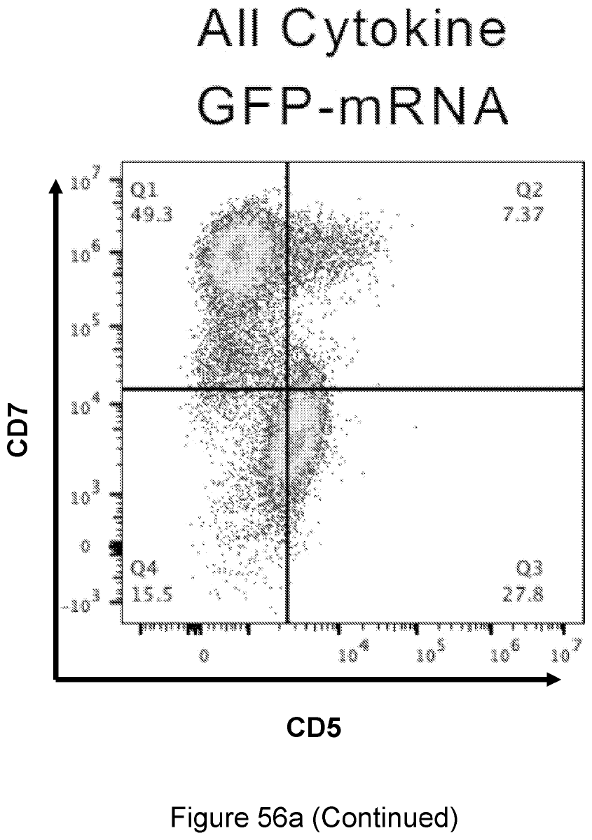

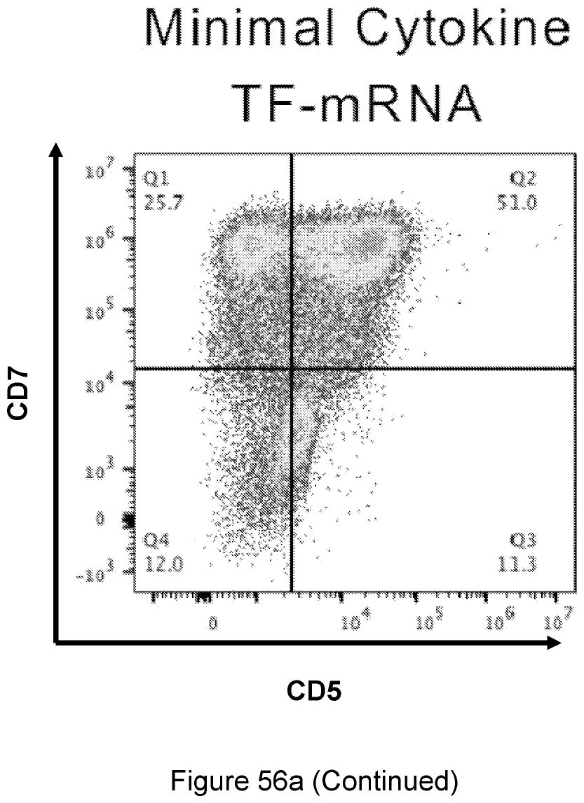

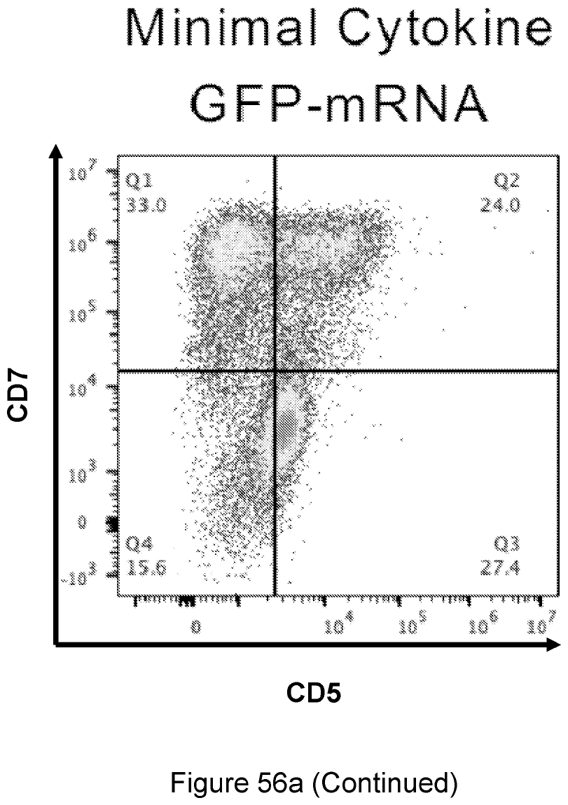

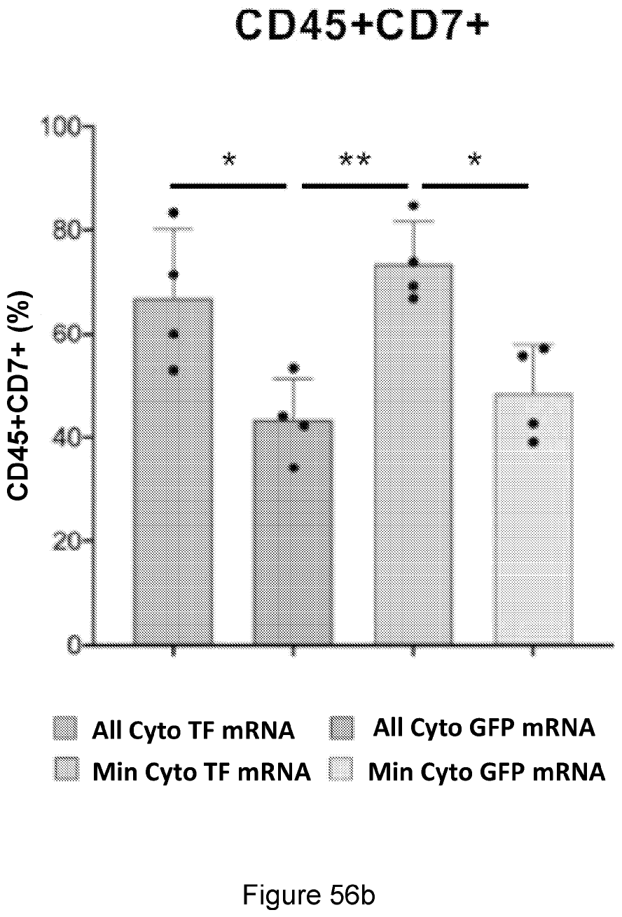

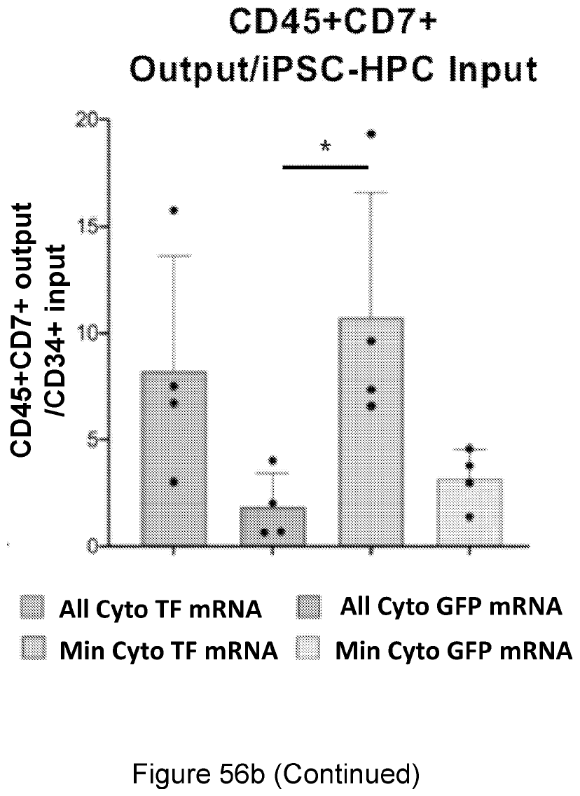

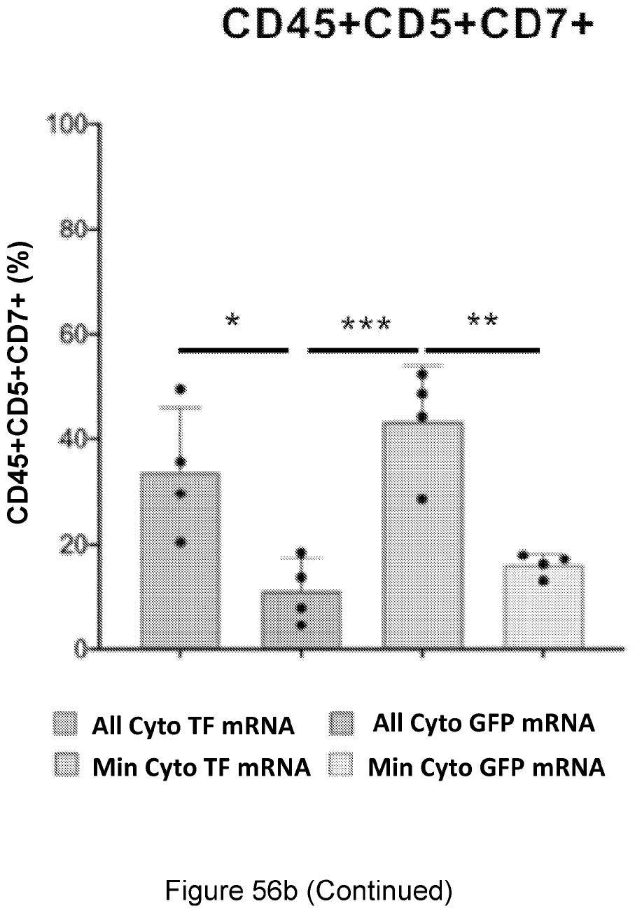

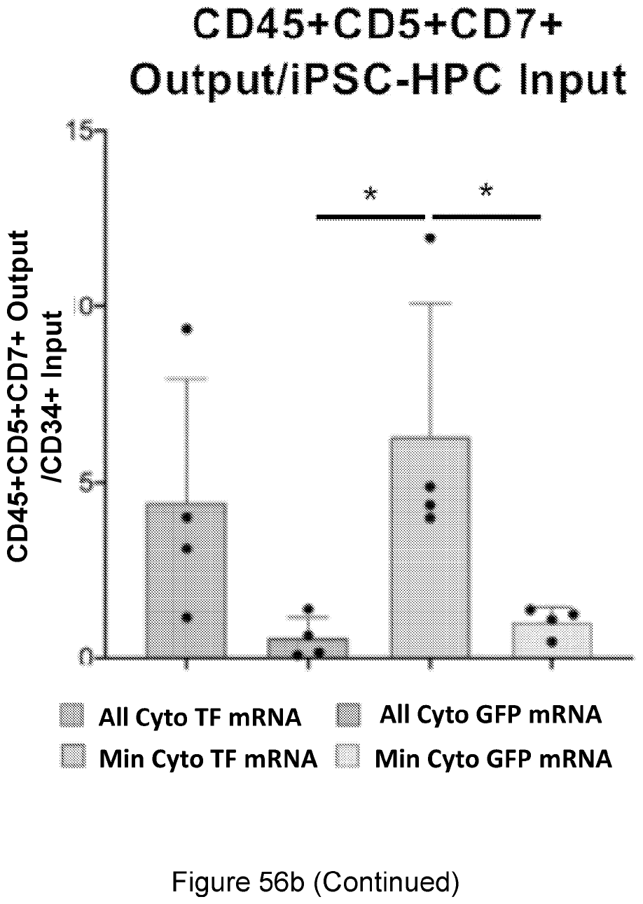

Next, we tried to differentiate the Day 9 iPSC-HPCs into Tcells in vitro. We only moved forward with differentiating the GFP and TF mRNA conditions given the lack of CFU colonies in the Control condition (FIG. 24). Using a commercially available kit from StemCell Technologies we differentiated the Day 9 GFP and TF mRNA cells under feeder-free conditions. After 14 days of differentiation, the frequency of T progenitors was assessed by analyzing CD5 and CD7 expression. While both had similar levels of CD5+ cells (FIG. 56A-B), the TF mRNA cells expanded more during the 14-day culture and thus produced a higher number of CD7+ cells. Similarly, the total number of CD5+CD7+ cells per CD34+ input was higher for the TF mRNA conditions compared to the GFP mRNA condition (FIG. 25C). The T cell differentiation was continued for another 14 days (28 days total) and cells were analyzed for CD4, CD8 and CD3 expression (FIG. 26). Although cells treated with TF mRNA produced a higher number of CD4+CD8+ and CD3+ cells compared to the GFP mRNA cells, neither of the conditions were efficient at making T cells defined by CD3, CD4 and CD8 expression.

### Day 13 iPSC-HPCs

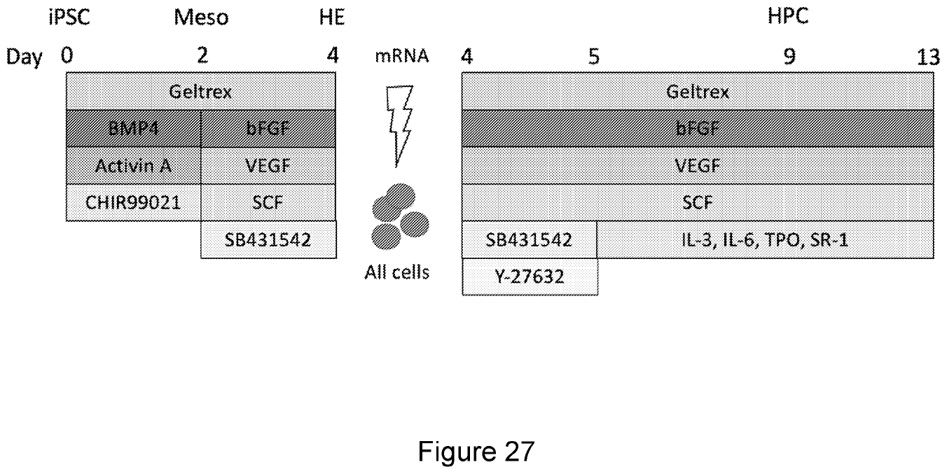

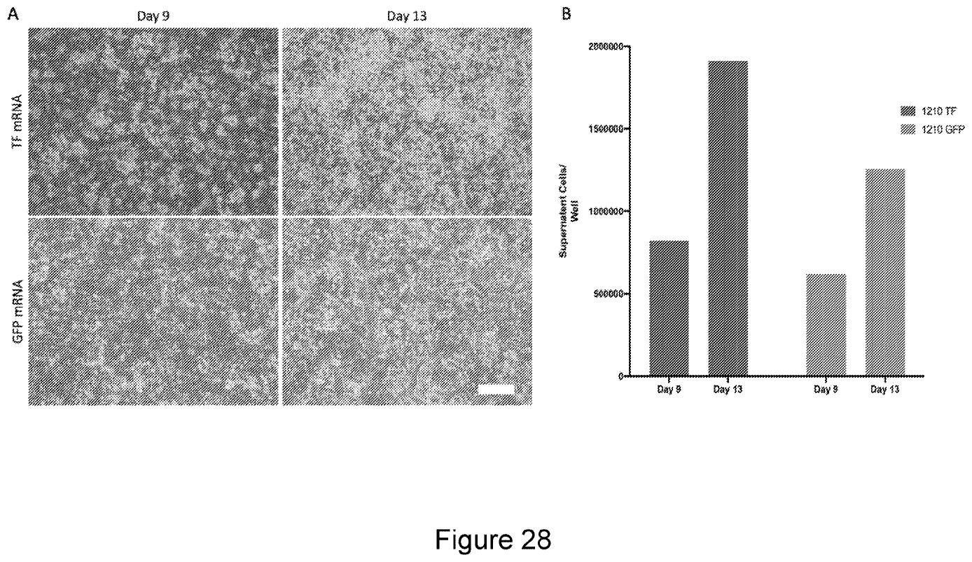

In order to improve the hematopoietic potential of the Day 9 iPSC-HPCs, we extended the length of differentiation from Day 9 to Day 13 (FIG. 27). While the number of CD34+ cells collected per one well of a 6-well plate on Day 9 for GFP and TF mRNA were similar (650k and 800k), the Day 13 TF mRNA resulted in significantly more CD 34+ cells per well compared to the GFP mRNA condition (1.9M vs 1.3M).

### Day 13 iPSC-HPC Clonogenic Output

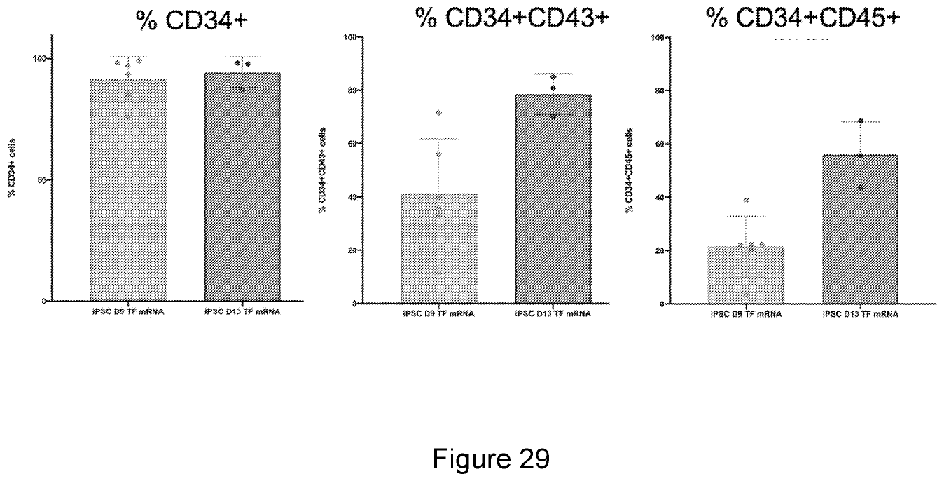

Comparing Day 9 and Day 13 TF mRNA conditions, the amount of CD34+ cells was similar but there was an increase in the amount of CD34+CD43+ and CD34+CD45+ cells for Day 13 TF mRNA cells (FIG. 29).

### Day 13 iPSC-HPC Clonogenic Output

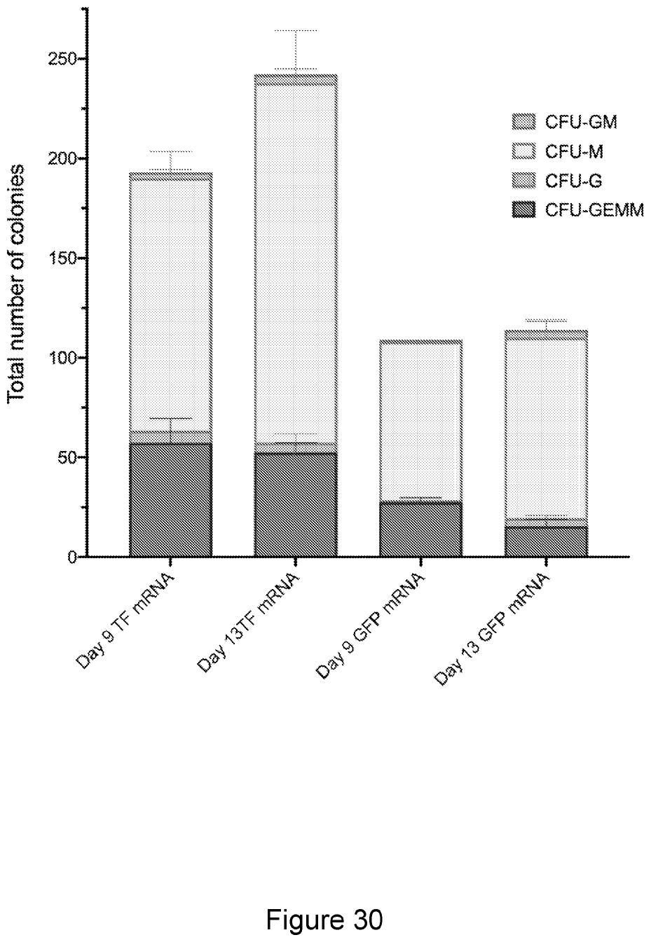

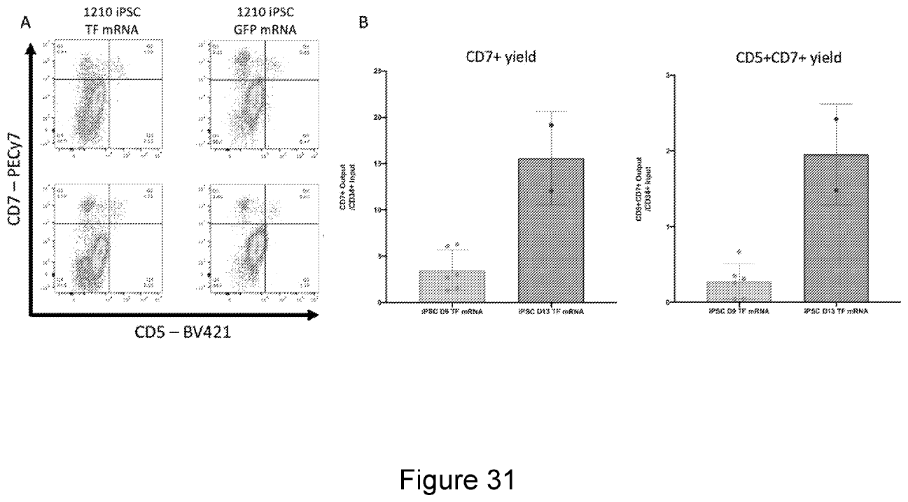

To assess the functionality of the Day 13 iPSC-HPCs we performed a CFU assay as was done with Day 9 cells (FIG. 24). The Day 13 TF mRNA cells generated more colonies than the Day 9 TF mRNA cells indicating a higher clonogenic potential (FIG. 30). The Day 9 GFP mRNA and Day 13 GFP mRNA treated cells had equal amounts of colonies formed.

### T Cells Generated From Day 13 iPSC-HPCs

Day 9 and Day 13 cells were used in a feeder-free T cell differentiation protocol. Similar levels of CD7+ and CD5+CD7+ were observed in GFP and TF mRNA treated cells regardless of Day 9 or Day 13. What did improve was the amount of CD7+ and CD5+CD7+ cells generated in the Day 13 TF mRNA condition compared to Day 9 TF mRNA cells, owing to the fact that the Day 13 cells were more proliferative.

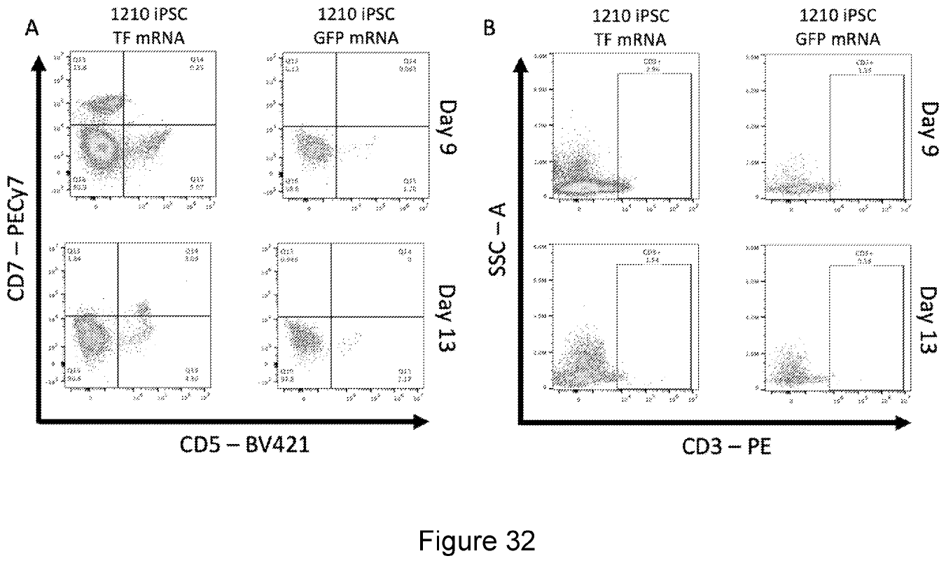

The T cell differentiation was continued for another 14 days (28 days total) and cells were analyzed for CD4, CD8 and CD3 expression (FIG. 32). As was the case with Day 9 cells in FIG. 26, the Day 13 GFP and TF mRNA displayed little to no CD4+CD8+ and CD3+ cells. We therefore conclude that although the 1210 iPSC Day 9 and Day 13 cells are capable of making T progenitor cells using either GFP or TF mRNA, they are unable to progress to a more mature T cell fate under current culture conditions.

### Making Ipsc-HPCs From T Cell Derived Ipsc Lines

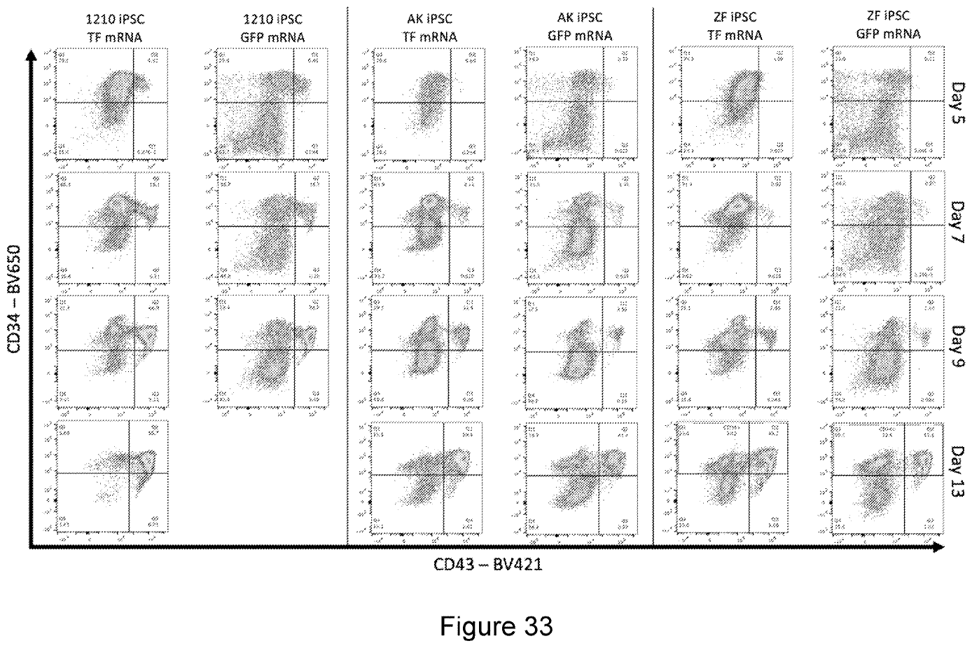

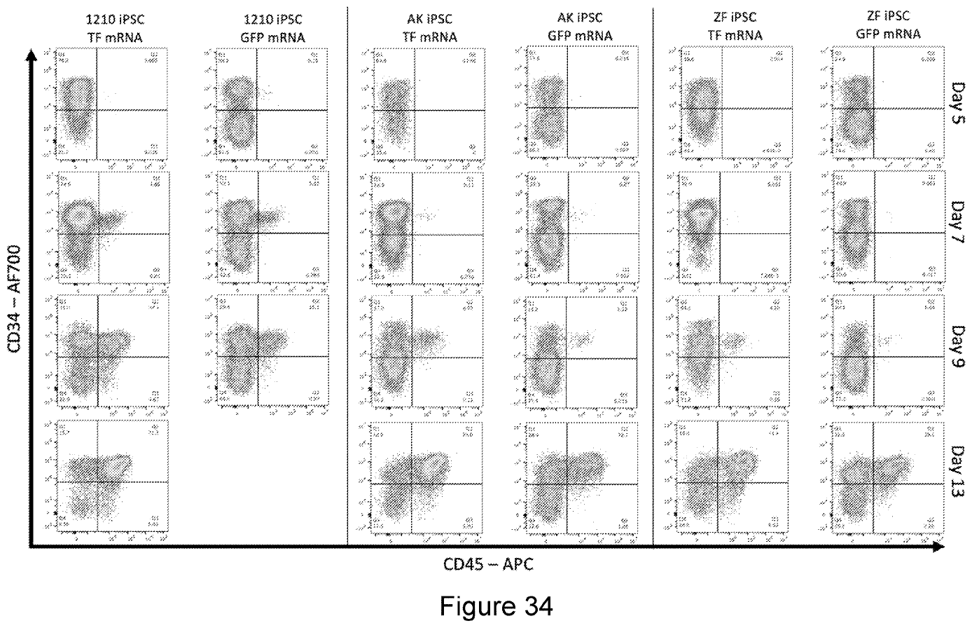

The efficiency of T cell differentiation is particularly poor for iPSC derived from somatic cells with a germline T cell receptor configuration (TCR) compared to T cell-derived iPSC harboring a pre-rearranged TCR. Using our 2DMD protocol we differentiated two T cell derived iPSC lines, AK and ZF, first into iPSC-HPCs and then into T cells. Cells were treated with either GFP or TF mRNA on Day 4 and cultured until Day 9 and Day 13 (FIG. 33). Both the AK and ZF line were able to generate CD34+ and CD34+CD43+ cells, although less efficiently than the 1210 line. On Day 9 there was a noticeable difference in the amount of CD34+ expression between GFP and mRNA treated cells for all three cell lines. On Day 13 this held true for the 1210 and AK iPSC lines but there was minimal difference between GFP and TF mRNA treated cells for the ZF line. Next, we looked at CD45 expression and saw the same trend as CD43 expression (FIG. 34). All three cell lines had higher CD34+CD45+ expression on Day 9 in the TF mRNA cells compared to the GFP mRNA treated cells. This was also true for the Day 13 time point except for the ZF line which had equal amounts of CD34+CD45+ between GFP and TF mRNA treated cells. This data indicates the 2DMD protocol is efficient at producing iPSC-HPCs in multiple cell lines.

### Making T Cells From Ak and Zf Ipsc-HPCs

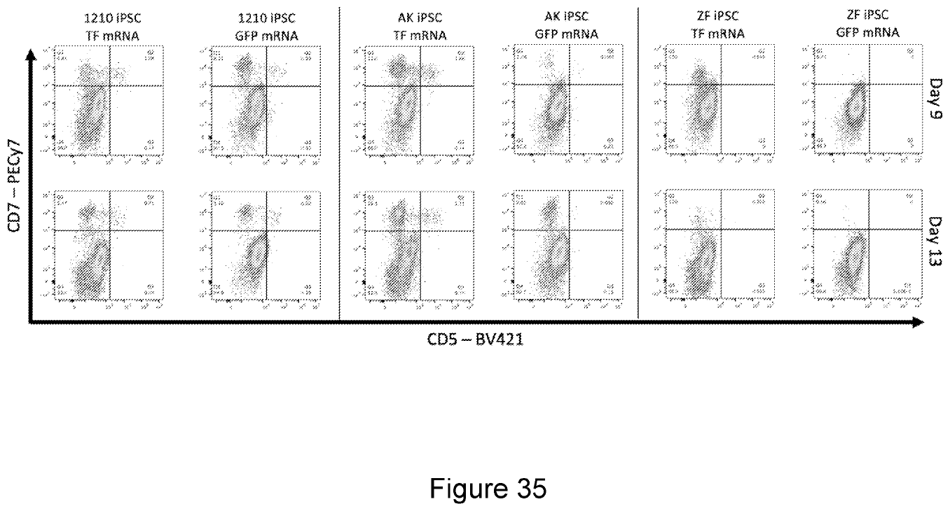

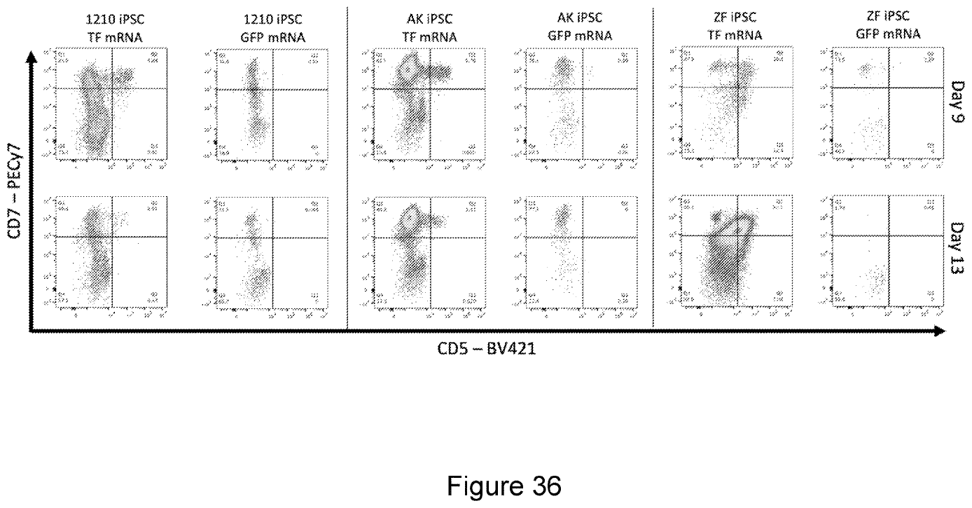

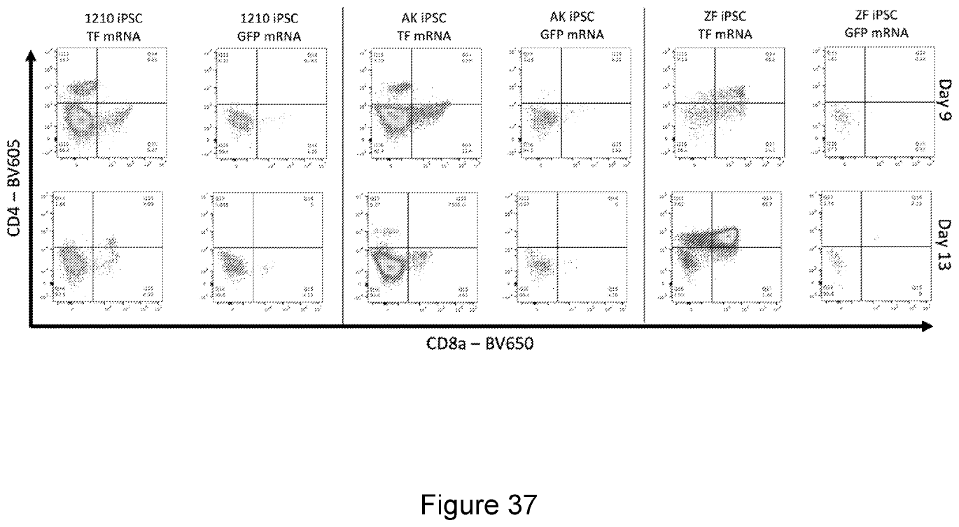

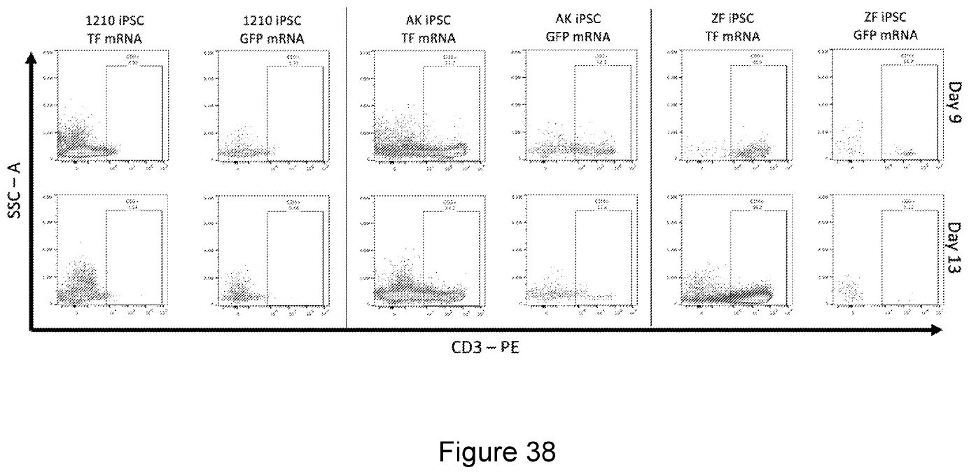

Day 9 and Day 13 iPSC-HPCs from all three cell lines were cultured in a 14-day feeder-free T cell differentiation protocol and then analyzed for CD5 and CD7 expression (FIG. 35). For both the AK and ZF iPSC-HPCs the TF mRNA condition produced more CD7+ and CD5+CD7+ compared to the GFP mRNA condition. The T cell differentiation was continued until Day 28 and again we analyzed the cells for CD5 and CD7 expression. While the Day 14 and Day 28 time points looked similar for the 1210 and AK iPSC-HPCs, the Day 28 ZF iPSC-HPCs had a large amount of CD5+CD7+ cells in the TF mRNA condition but little in the GFP mRNA condition. Looking at CD4 and CD8 expression we saw little in the 1210 and AK iPSC-HPC conditions but the ZF iPSC-HPCs (both Day 9 and Day 13 starting cell sources) produced a large amount of CD4+CD8+ cells. Lastly, as expected, both the AK and ZF iPSC-HPCs had a high amount of CD3 expression compared to the 1210 line (FIG. 38). The TF mRNA conditions produced a higher amount of CD3+ cells compared to the GFP mRNA cells for all three cell lines.

### ECM Optimization for in Vitro Feeder-Free T Cell Differentiation

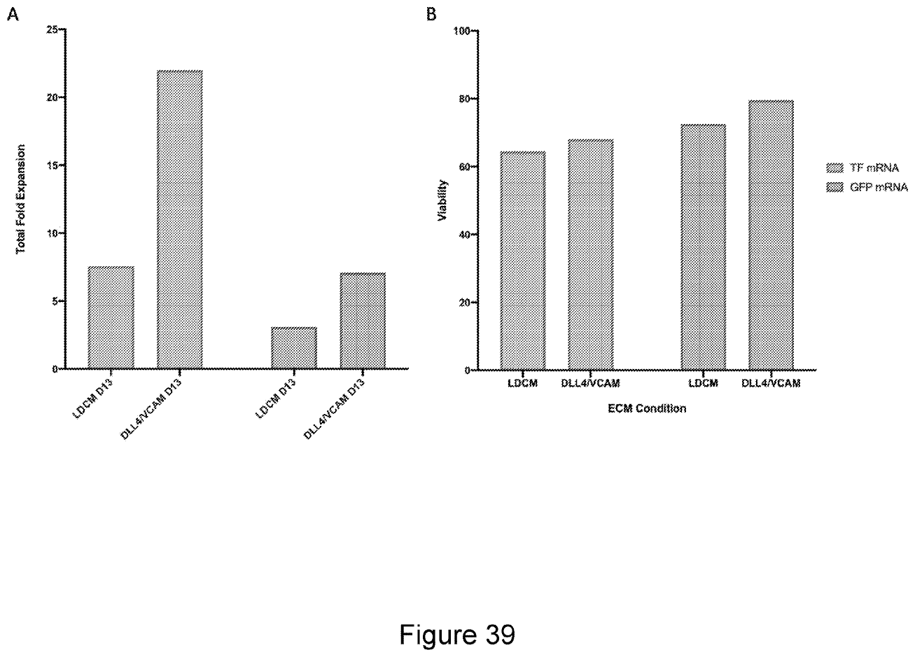

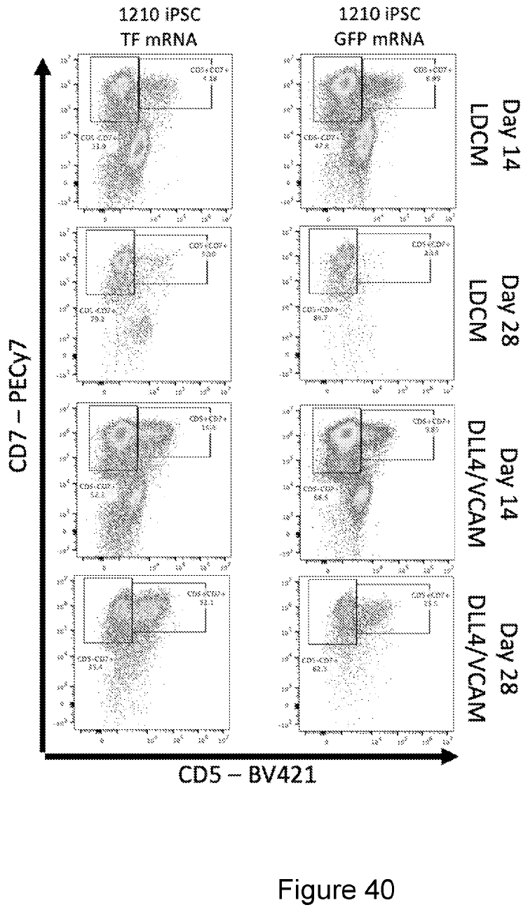

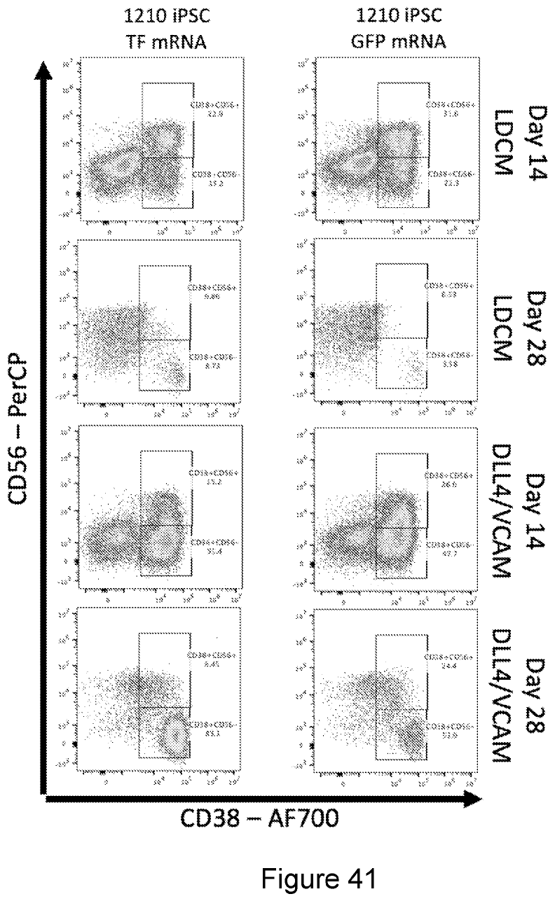

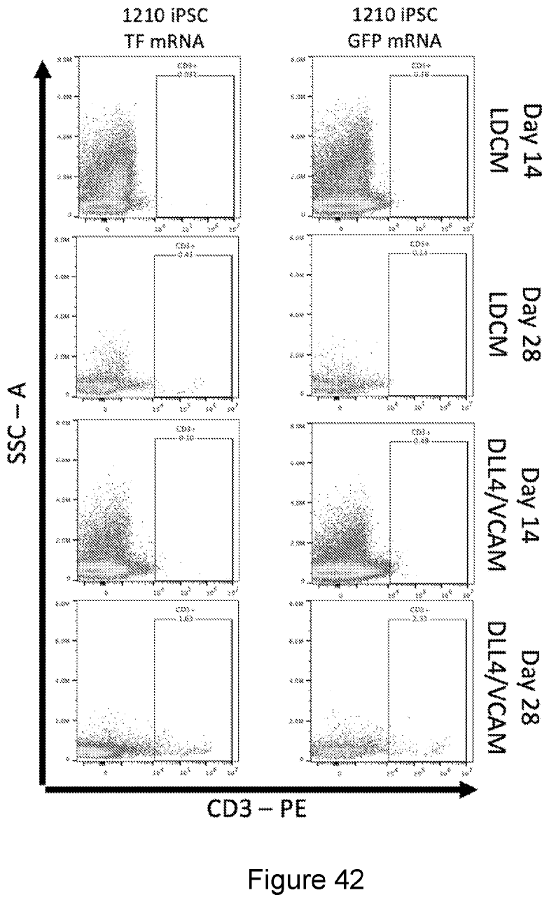

During development T cells need Notch signaling to differentiate into mature T cells. The T cell kit from StemCell Technologies provides a lymphoid differentiating coating material (LDCM) and T cell maturation media. They do not disclose what is in the coating material or media. We hypothesized that the LDCM contained DLL4, a Notch ligand important for T cell development. Due to low CD7+ and CD5+CD7+ T progenitor cells in our 1210 iPSC line, we wanted to increase the concentration of DLL4 coating the plate to see if that would improve the frequency of T progenitors. In addition to DLL4, several papers have reported that VCAM-1 acts synergistically with DLL4 to provide a stronger Notch signal. Therefore, we coated plates with either LDCM or a mixture of DLL4/VCAM-1 and then proceeded with an in vitro feeder-free T cell differentiation using the StemCell Technologies T cell maturation media using 1210 iPSC-HPCs from Day 13 GFP and TF mRNA conditions. After 14 days of T cell differentiation the DLL4/VCAM coating produced a higher amount of cells compared to LDCM for both the GFP and TF mRNA conditions (FIG. 39). Cell viability was similar for the two coating materials.

### Reduced Cytokine Generation of HSPCs

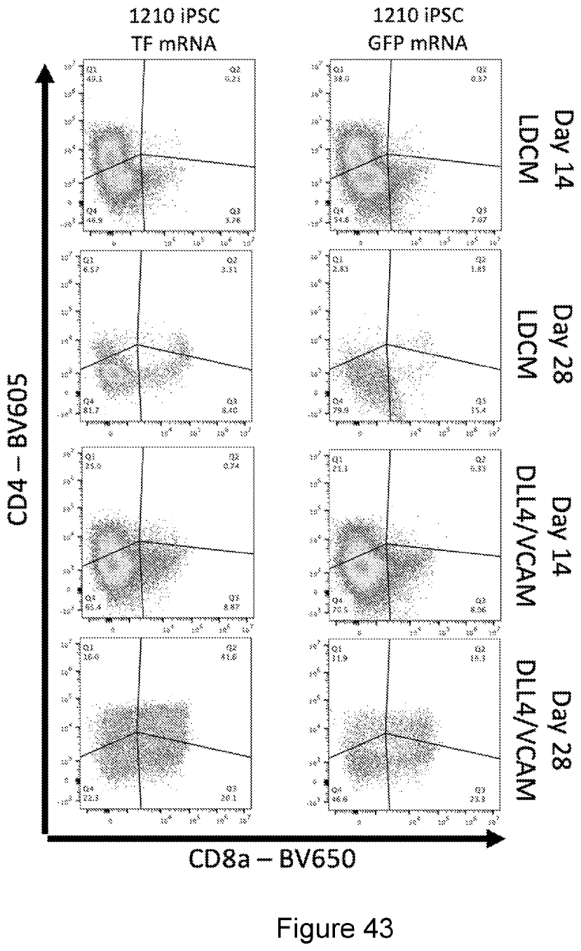

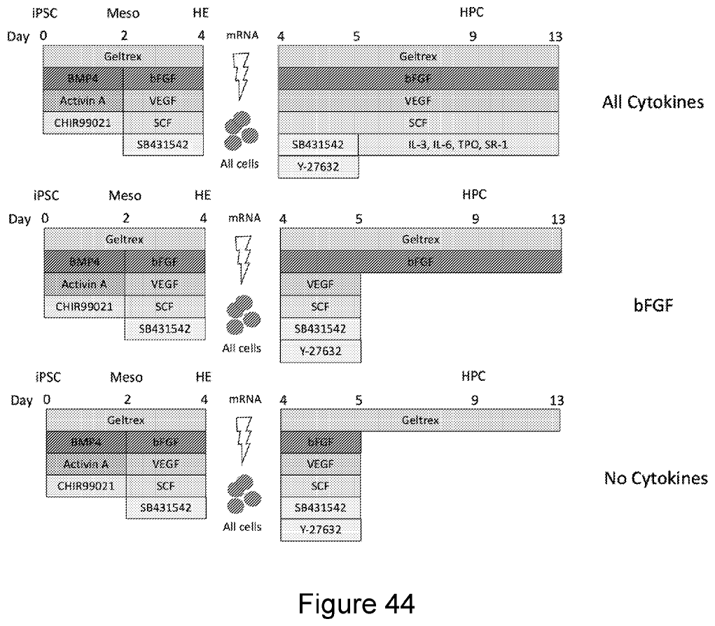

The inventors discovered that delivery of mRNA, by electroporation, encoding the transcription factors ERG, LCOR, HOXA5, HOXA9, and RUNX1 at about day 4 of culture allowed for generation of HSPCs with minimal cytokines in the culture medium from about day 5 to about day 13 (FIGS. 43 and 44), i.e., without the addition of IL-3, IL-6, TPO or SR-1.

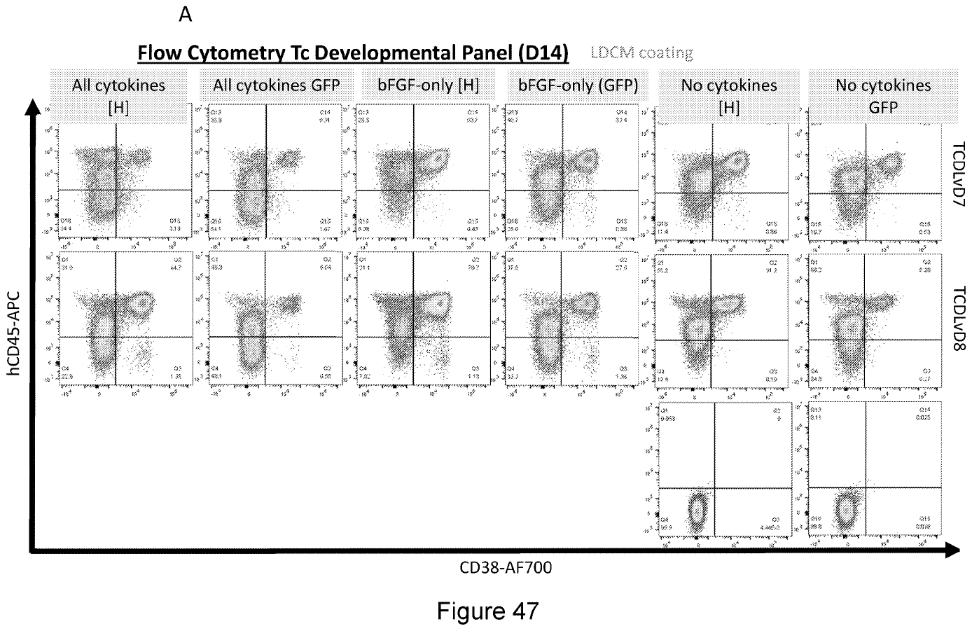

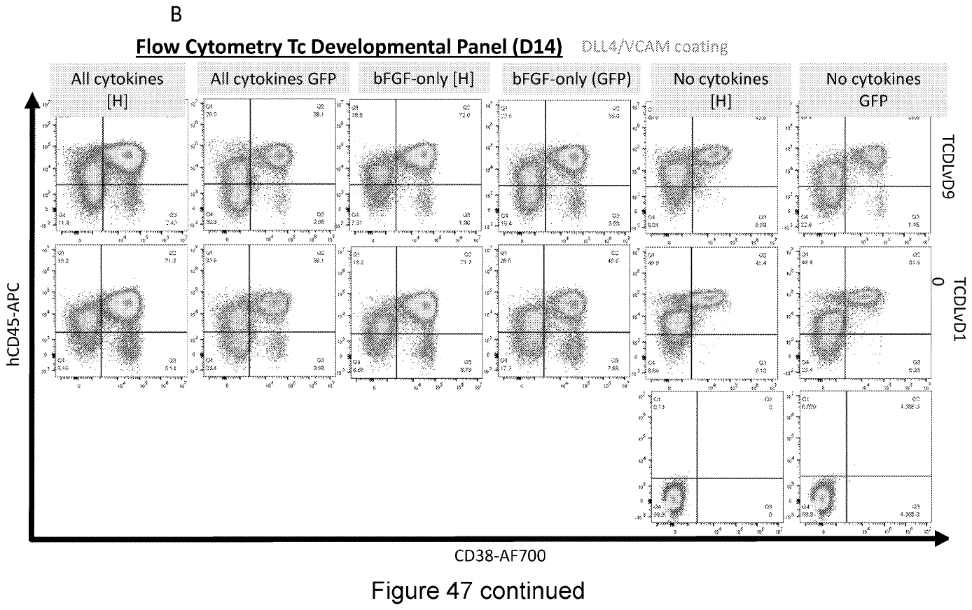

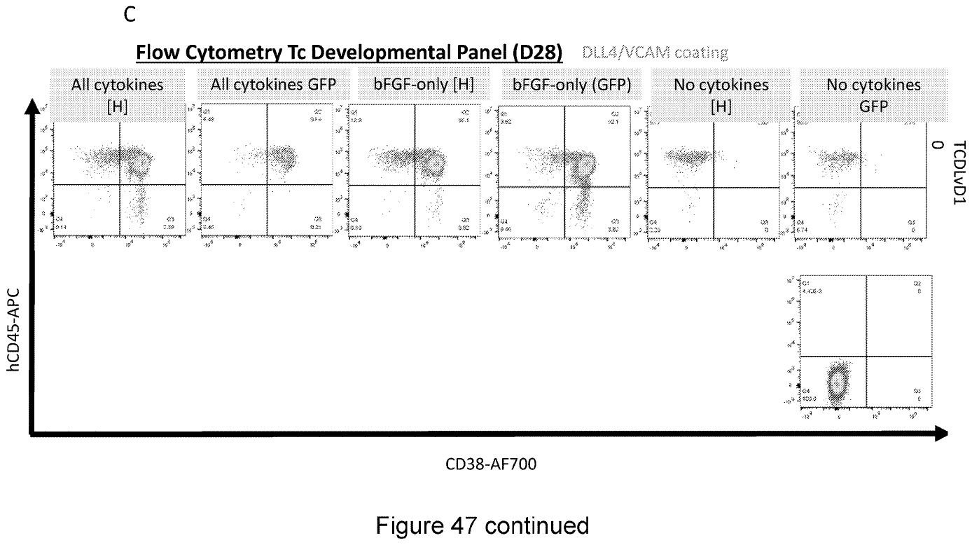

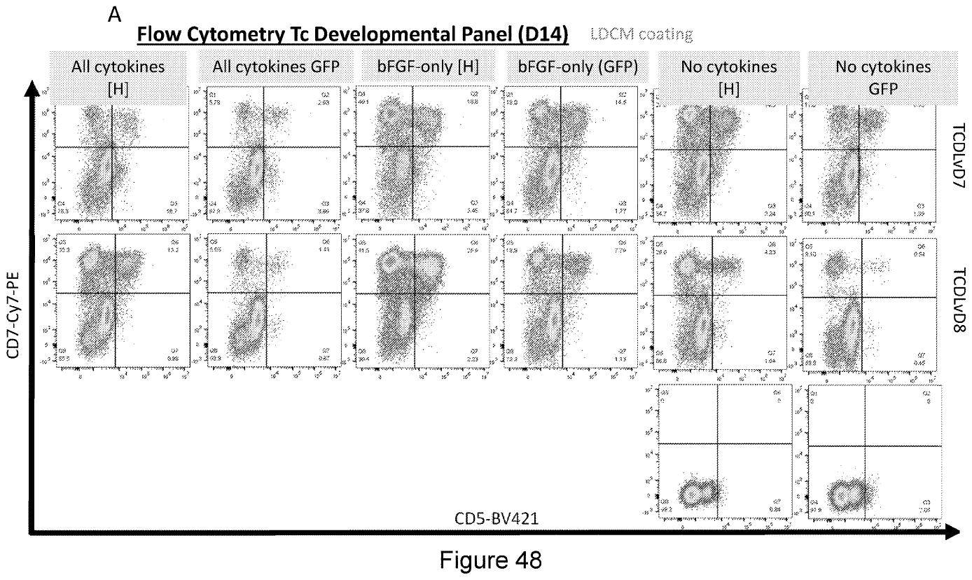

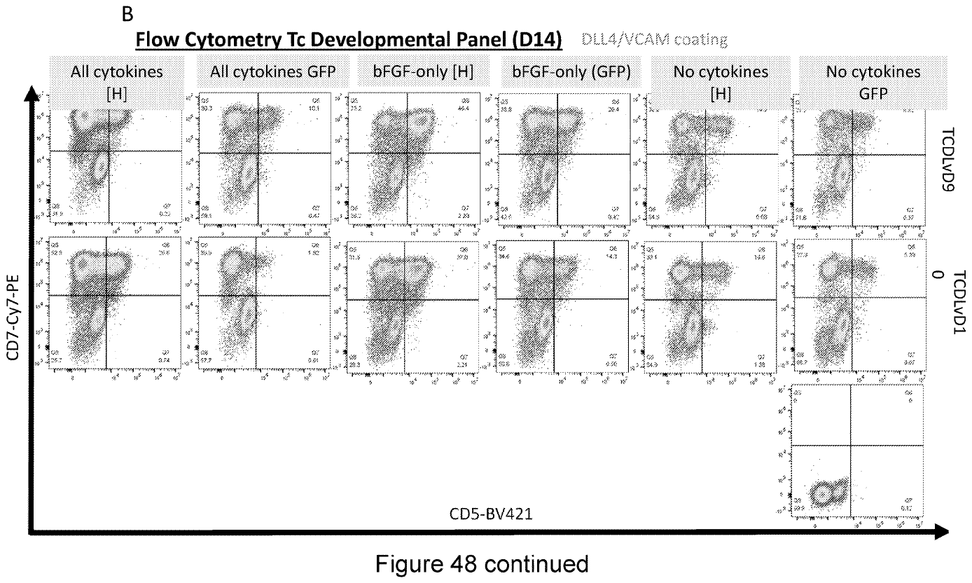

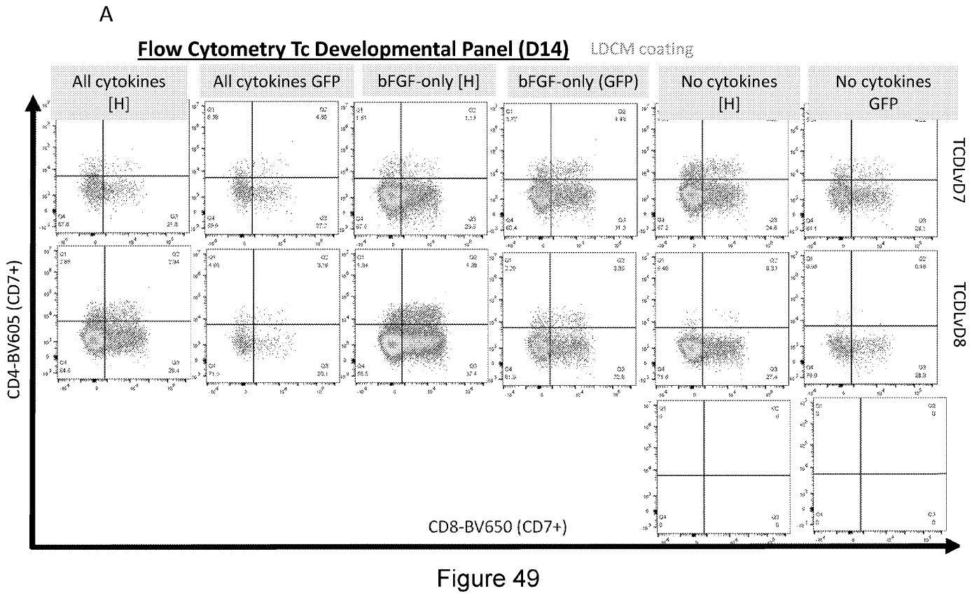

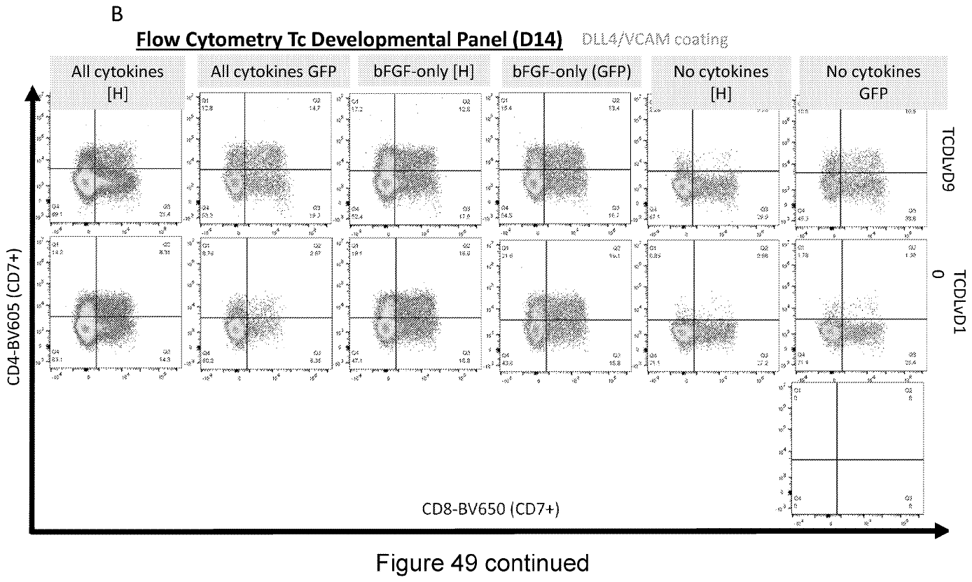

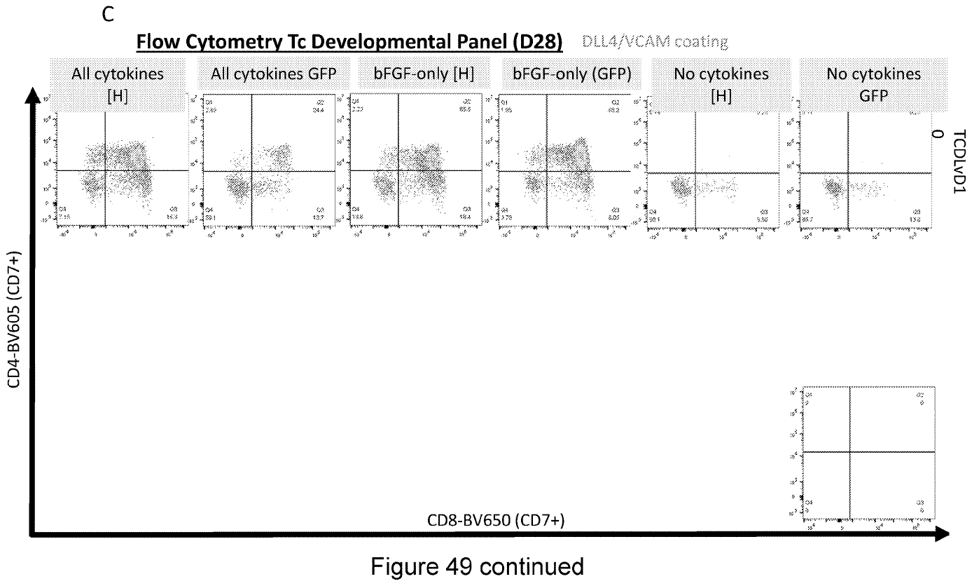

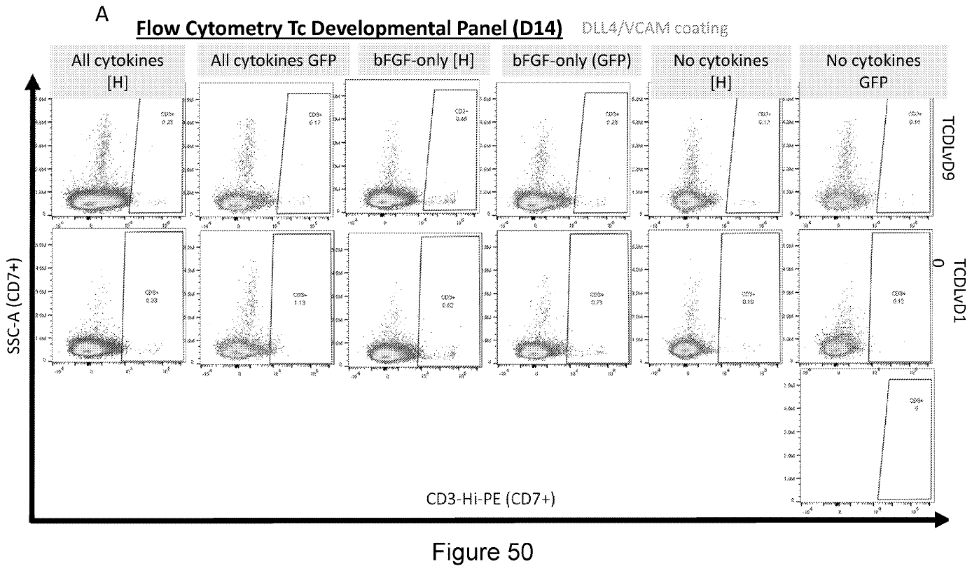

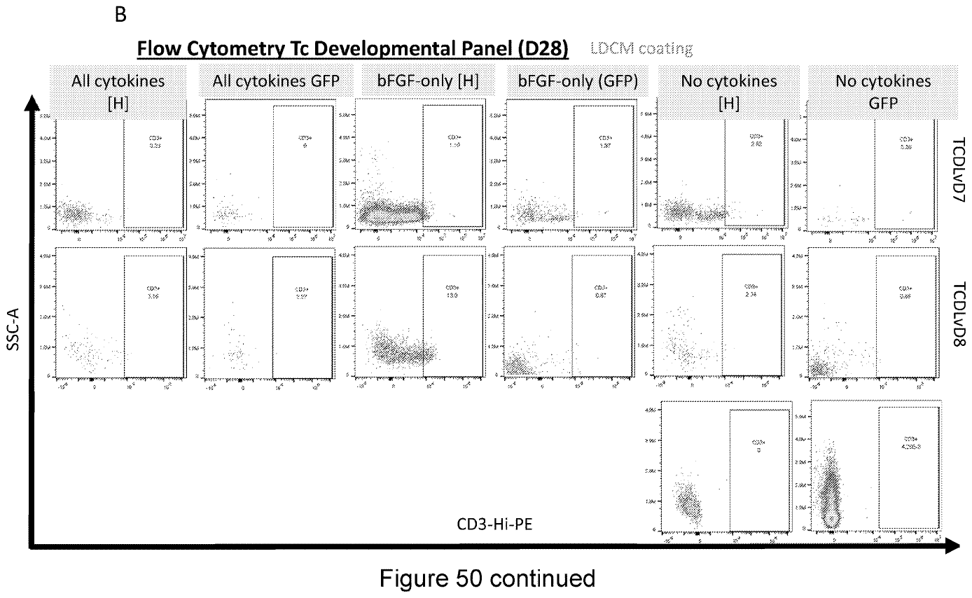

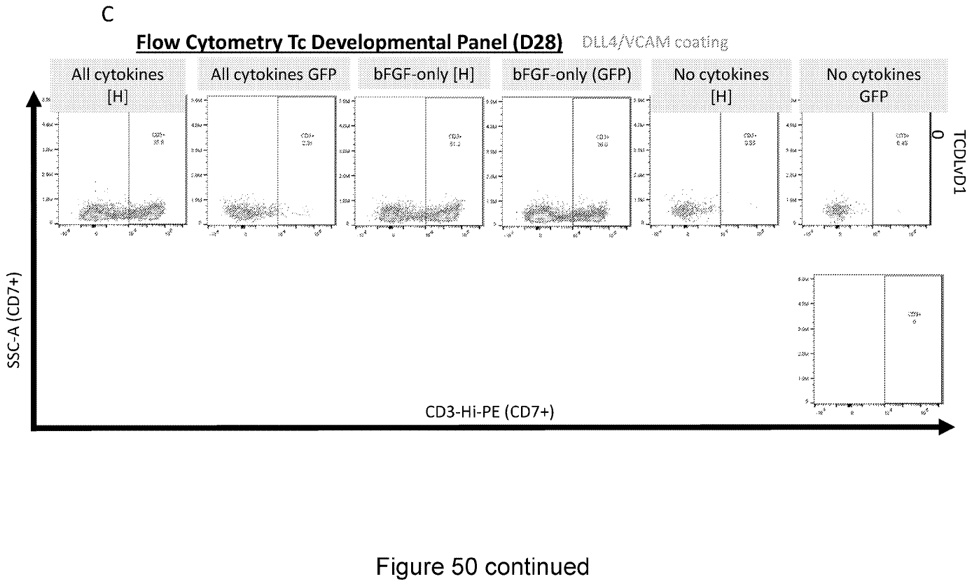

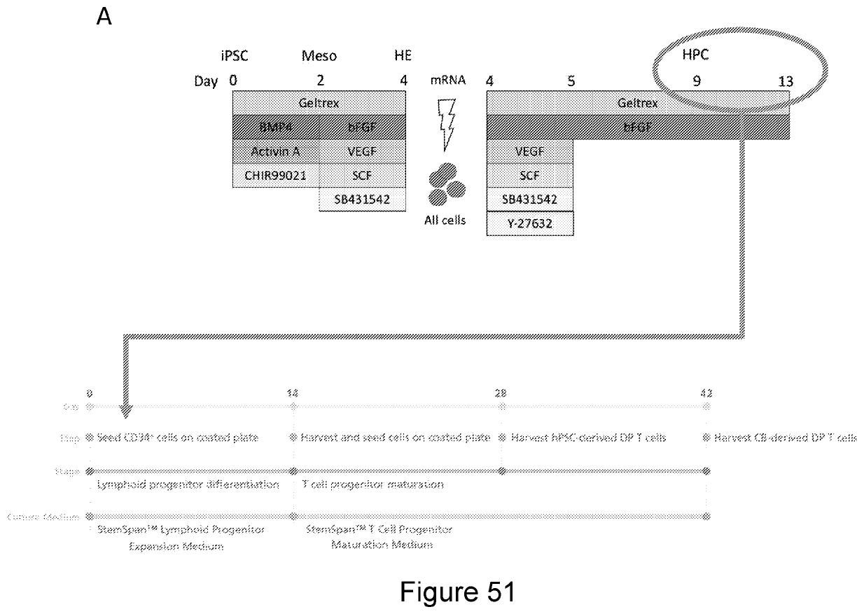

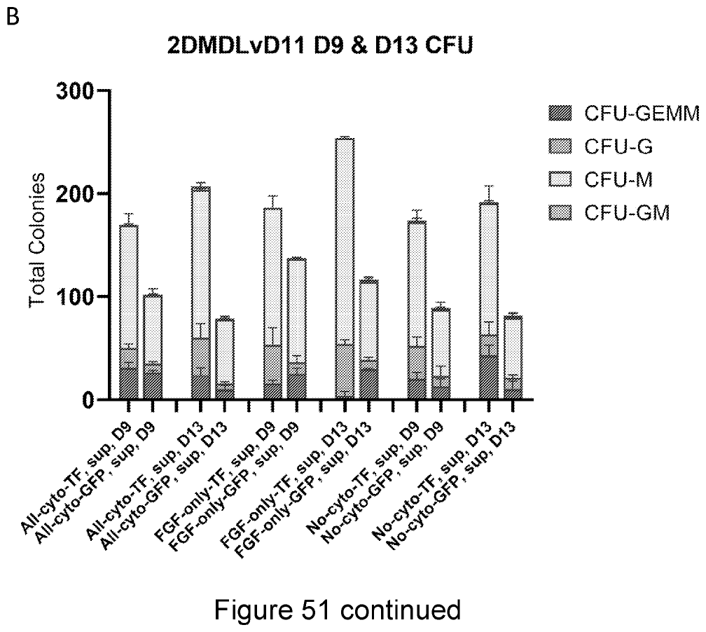

Two alternative approaches allow for the differentiation of iPSC without the use of IL-3, IL-6, TPO or SR-1. A minimal-cytokine or cytokine-free method does not expose the cells to any growth factors or cytokines starting at day 5 in culture. An bFGF-only approach exposes cells to only bFGF starting at day 5 in culture (FIG. 43). Starting at day 7 and continuing to day 13 a c-kit+bifurcation appears using these two approaches. As shown in FIG. 45, c-kit (CD117) expression is increased in FGF-only and minimal-free produced cells compared to control samples. FGF-only and cytokine-free methods are also able to produce myeloid and lymphoid cells (FIGS. 47-51).

### Example 2—Transfer of Ipsc Derived HSPCs to a Subject in Need Thereof

The disclosed methods may be used to differentiate HSPCs from iPSCs made from a subject's cells. Briefly, suitable cells may be collected to differentiate iPSCs from a subject, e.g., fibroblasts, peripheral blood mononuclear cells (PBMCs), kidney cells, keratinocytes, T cells, etc, and dedifferentiated into iPSCs using methods known in the art. See, for example, Takahashi et al. “Induction of pluripotent stem cells from adult human fibroblasts by defined factors” Cell. 2007 November 30; 131(5):861-72, which is incorporated by reference herein in its entirety. Next, iPSCs are cultured for about 2 days with bone morphogenetic protein 4 (BMP4), activin A, and CHIR99021, cultured for about 2 days with basic fibroblast growth factor (bFGF), vascular endothelial growth factor (VEGF), stem cell factor (SCF), and SB431542, electroporated with mRNA encoding the transcription factors ETS-related gene (ERG), ligand-dependent corepressor (LCOR), homeobox A5 (HOXA5), homeobox A9 (HOXA9), and runx family transcription factor 1 (RUNX1), and cultured in the presence of bFGF, VEGF, SCF, SB431542, and Y-27632 to generate CD34⁺ HSPCs that are autologous to the subject.

In some situations, the subject will require ablation of their existing bone marrow prior to engraftment of new HSPCs into the bone marrow compartment. Suitable methods of ablating bone marrow are known in the art. Accordingly, the subject may then be administered, e.g., administered intravenously, an amount of autologous iPSC-derived HSPCs to engraft into the subjects bone marrow and restore hematopoiesis in the subject.

### Example 3—Adoptive Transfer of Ipsc Derived T Cells to a Subject in Need Thereof

The disclosed methods may be used to differentiate HSPCs from iPSCs made from a subject's cells, then the HSPCs may be differentiated into T cells. Briefly, suitable cells may be collected to differentiate iPSCs from a subject, e.g., fibroblasts, peripheral blood mononuclear cells (PBMCs), kidney cells, keratinocytes, and, T cells, e.g., tumor infiltrating lymphocytes, virus-specific T cells, and dedifferentiated into iPSCs using methods known in the art. See, for example, Iriguchi et al. “A clinically applicable and scalable method to regenerate T-cells from iPSCs for off-the-shelf T-cell immunotherapy” Nat. Commun. 2021 Jan. 18;12(1):430, which is incorporated by reference herein in its entirety. Next, iPSCs are cultured for about 2 days with bone morphogenetic protein 4 (BMP4), activin A, and CHIR99021, cultured for about 2 days with basic fibroblast growth factor (bFGF), vascular endothelial growth factor (VEGF), stem cell factor (SCF), and SB431542, electroporated with mRNA encoding the transcription factors ETS-related gene (ERG), ligand-dependent corepressor (LCOR), homeobox A5 (HOXA5), homeobox A9 (HOXA9), and runx family transcription factor 1 (RUNX1), and cultured in the presence of bFGF, VEGF, SCF, SB431542, and Y-27632 to generate CD34⁺ HSPCs that are autologous to the subject.

Next the HSPCs may be differentiated into T cells by culturing the HSPCs in the presence of delta like canonical notch ligand 4 (DLL4) and, optionally, vascular cell adhesion molecule (VCAM) to differentiate the HSPCs into T cells. The T cells may be expanded by methods known in the art including culturing with one or more of interleukin 2, 7, and 15. The T cells may then be transferred, e.g., intravenously, to a subject in need of adoptive T cell transfer, e.g., a subject in need of treatment for a malignancy by adoptive transfer of autologous tumor infiltrating T cells.

### Example 4—Hematopoietic Transcription Factor mRNA in the Absence of Hematopoietic Cytokines Yields iPSC-CD34 Cells With Increased T Cell Potential

Due to the complexity of T cell development in vivo, current in vitro protocols for the generation of T cells from iPSCs (iPSC-T cells) often rely on feeder systems expressing notch ligands1 or iPSC lines derived from primary T cells that already harbor a prearranged T cell receptor (TCR).2 The development of stromal-free iPSC-T cell protocols are potentially more suitable for the generation of cell types that may be utilized in the clinic. Additionally, TCR diversity can be increased by utilizing non-T cell derived iPSC lines. Here we demonstrate that lymphoid potential of iPSC-derived hematopoietic progenitor cells (iPSC-HPCs) can be increased by the expression of hematopoietic transcription factors during the hemogenic endothelial stage of differentiation. Crucially, while this has been demonstrated using constitutive expression from viral vectors, or with doxycycline inducible systems, this work achieves comparable outcomes by transient expression of hematopoietic transcription factors delivered via mRNA. These studies provide an improved platform for in vitro production of iPSC-T cells with the potential application for therapy and basic biological studies.

### Objectives:

Transiently Express Tf-mrna (ERG, HoxA5, HOXA9, LCOR, and RUNX1) During the differentiation of a fibroblast-derived iPSC line to yield hematopoietic progenitor cells with increased lymphoid potential;

Compare the effects of All Cytokine and Minimal Cytokine media during the iPSC-HPC differentiation protocol; and
Differentiate Day 13 iPSC-HPCs into T cells using a feeder-free protocol.

### Methods

T cell differentiation. 24-well plates are coated with 15 μg/mL DLL4 and 2.5μg/mL VCAM-1 for 2 hours at room temperature or overnight at 4 °C. 4 Day 13 iPSC-HPCs are seeded at a density of 50,000 cells/well and cultured with Stemcell Technologies Lymphoid Progenitor Expansion Supplement for 14 days to generate T progenitor cells. On Day 14 500,000 iPSC-T progenitor cells are re-seeded onto fresh DLL4/VCAM-1 and cultured for an additional 14 days in Stemcell Technologies T cell Progenitor Maturation Supplement to generate CD4+CD8+ cells. Umbilical cord blood (UCB) UCB-CD34 cells are seeded at 5,000 on Day 0 and 50,000 on Day 14. They are differentiated for 42 days (UCB-T cells).

TCR repertoire analysis. gDNA was collected from Day 28 iPSC-T cells and subjected to TCRB sequencing using AmpliSeq for Illumina TCR beta-SR Panel (Illumina, Inc). The data was then analyzed using MiXCR software (MiLaboratories, Inc).

### Conclusions

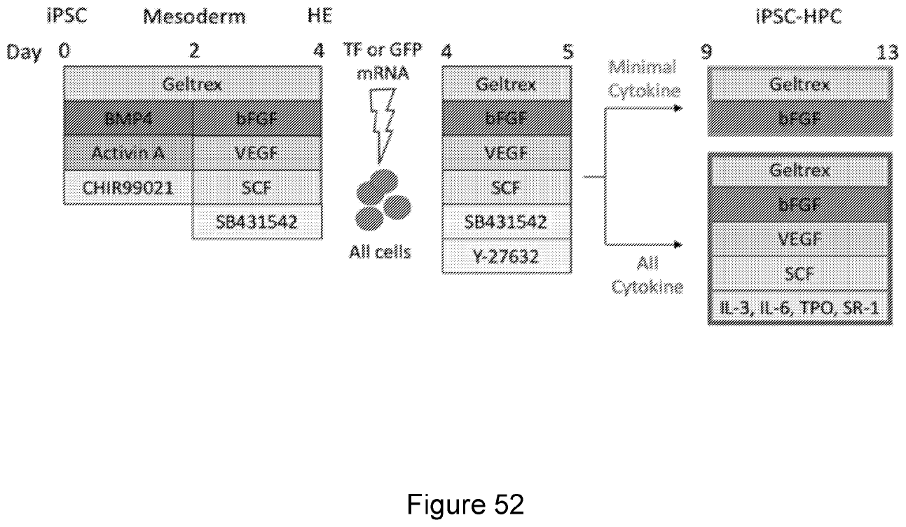

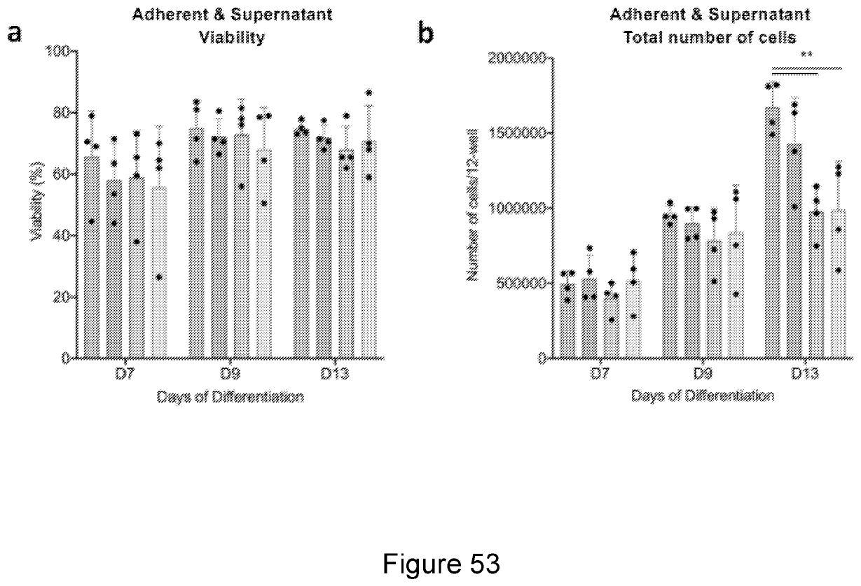

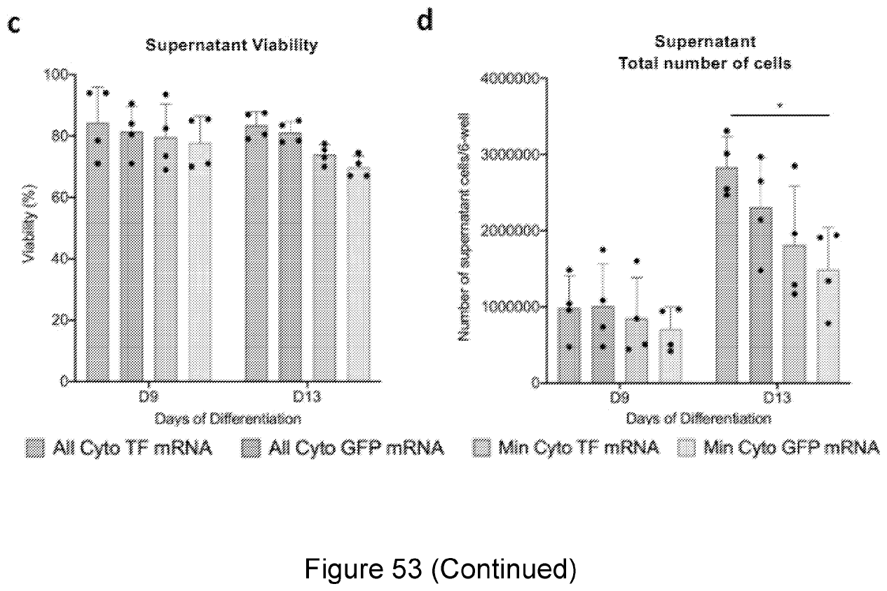

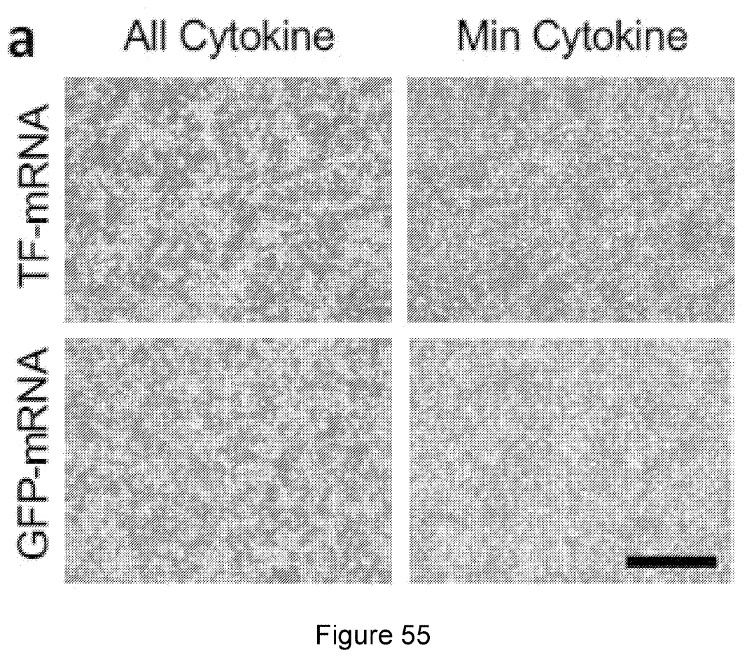

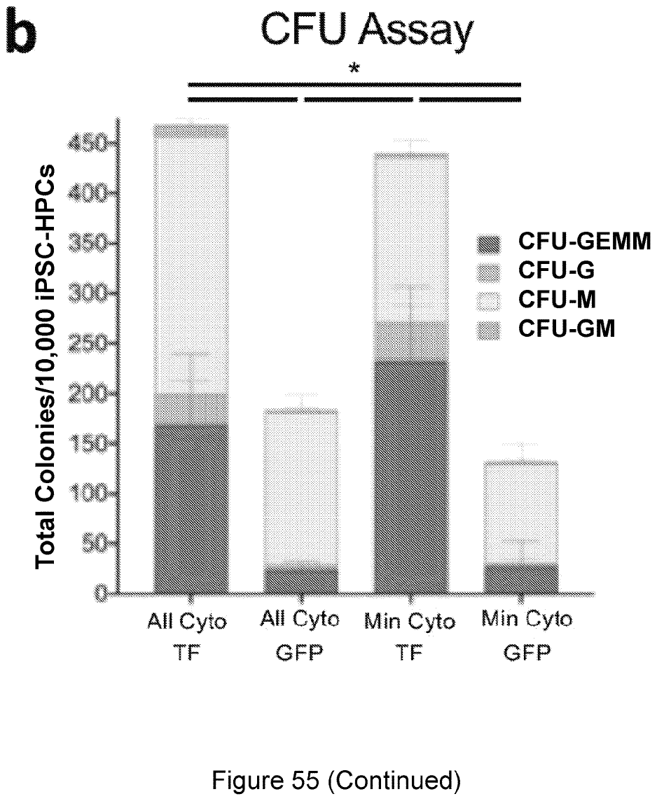

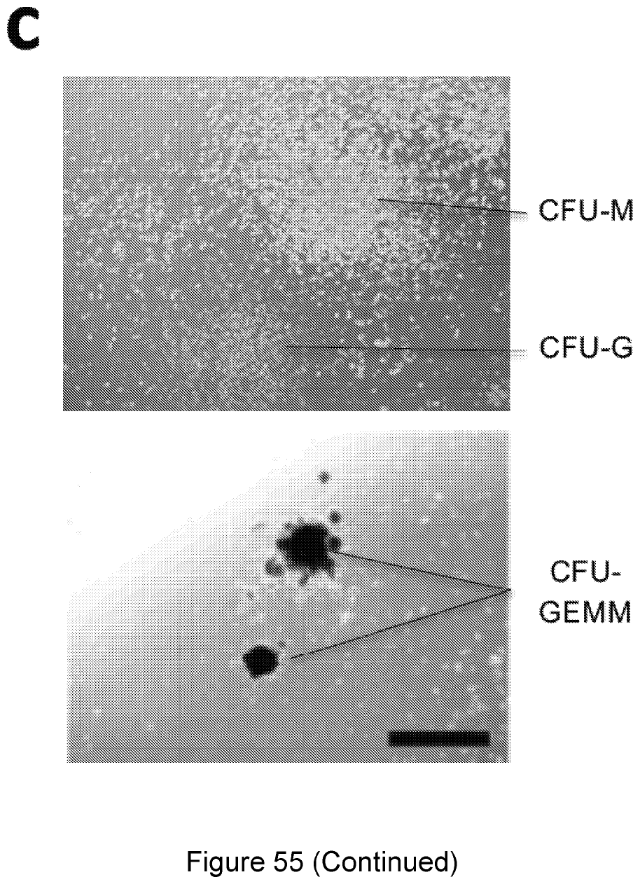

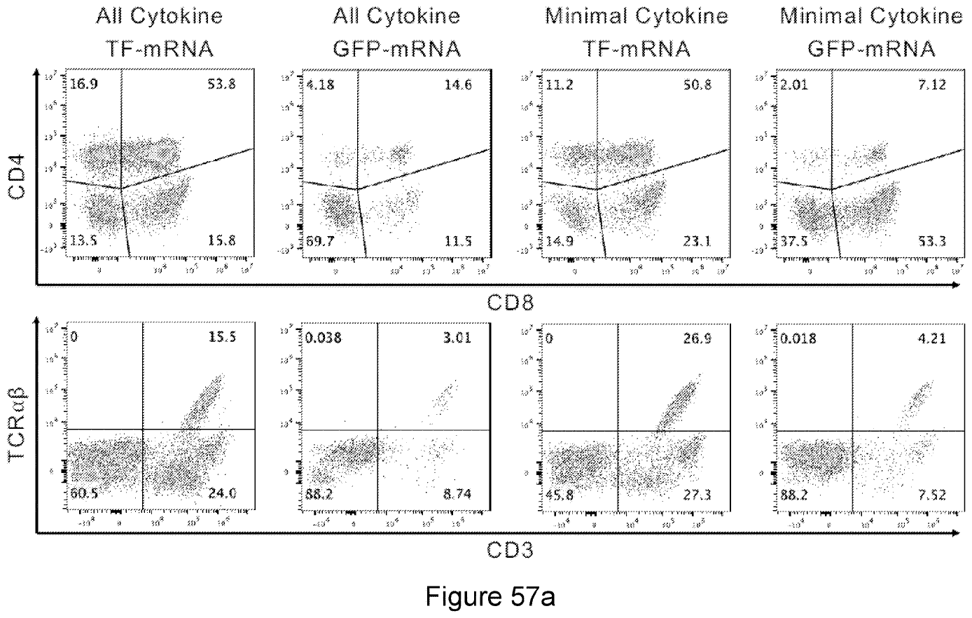

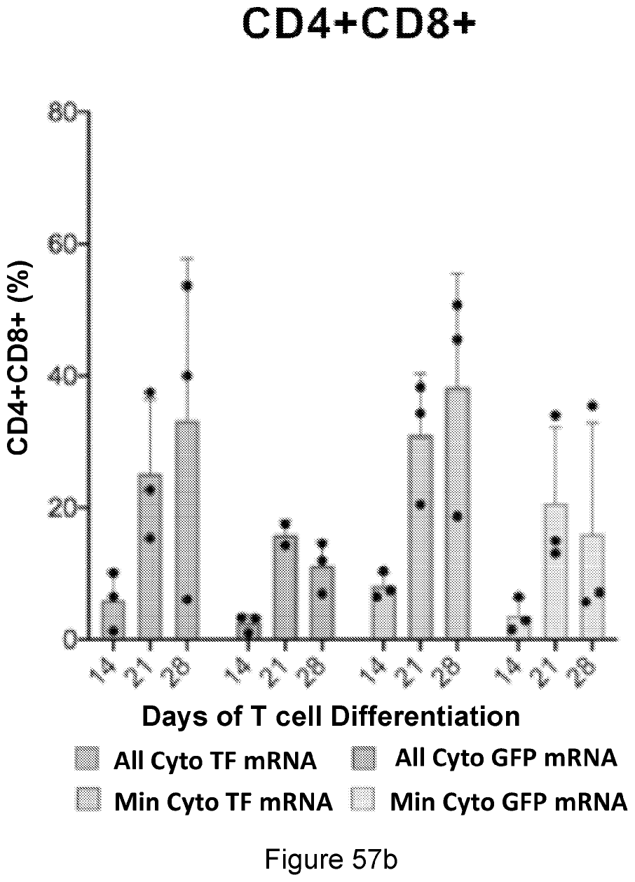

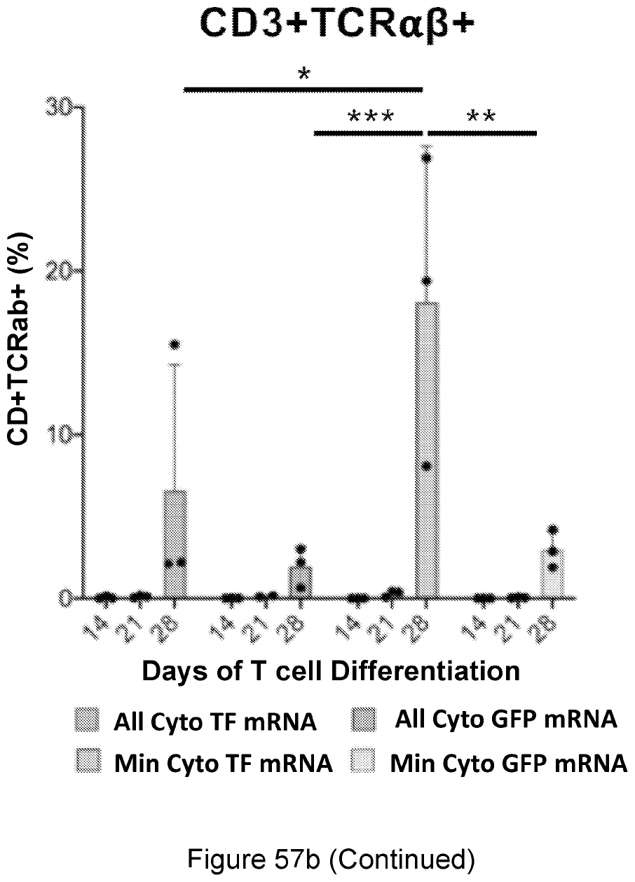

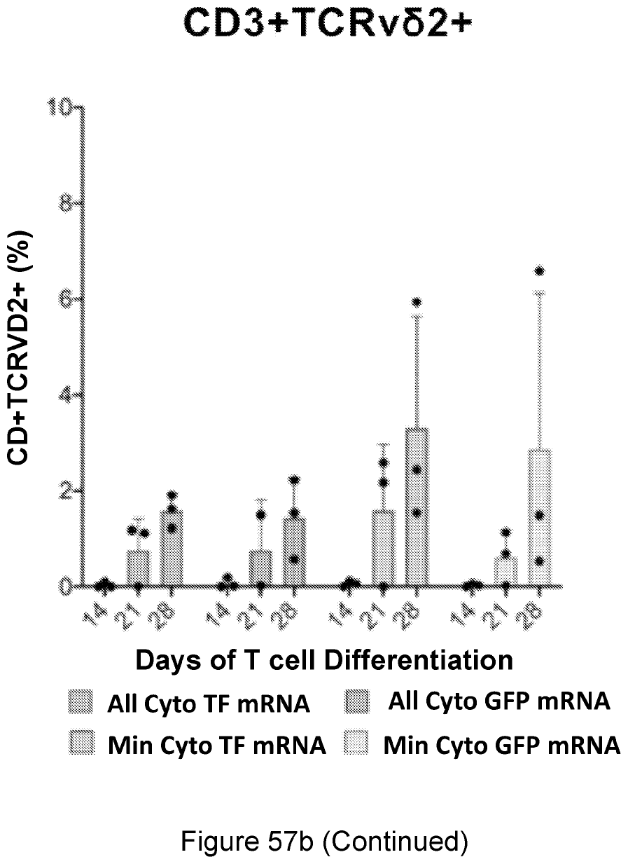

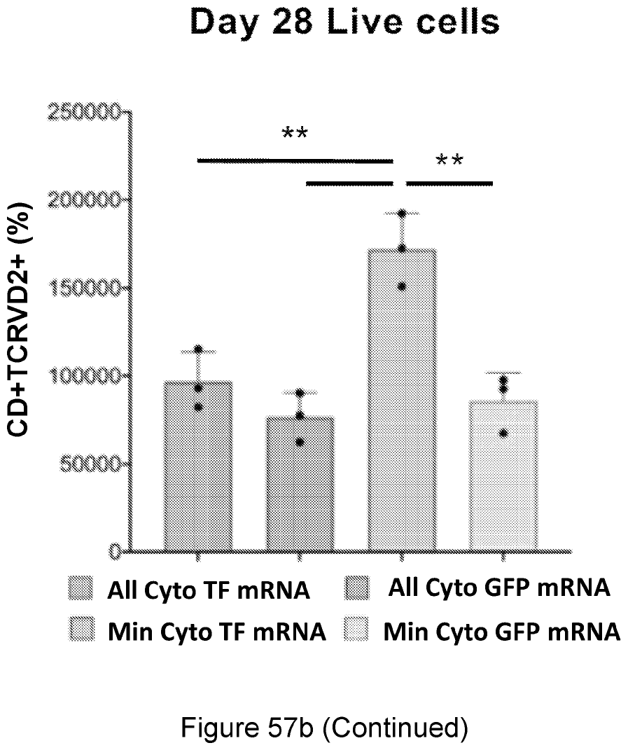

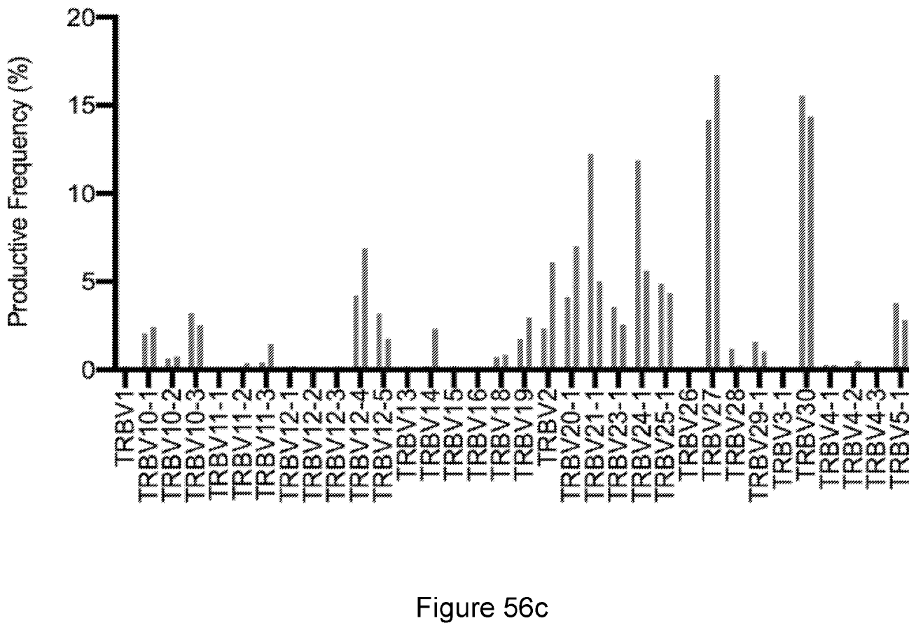

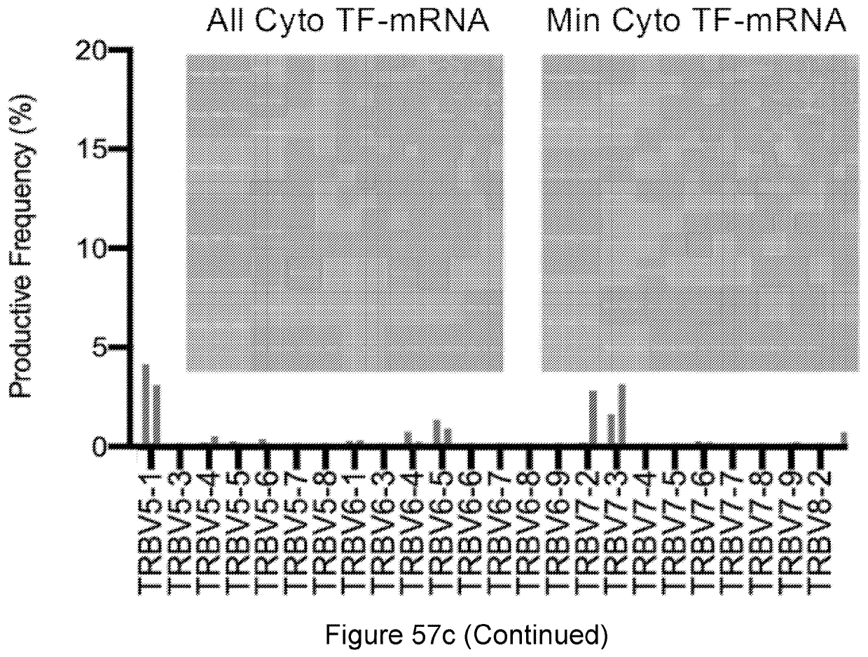

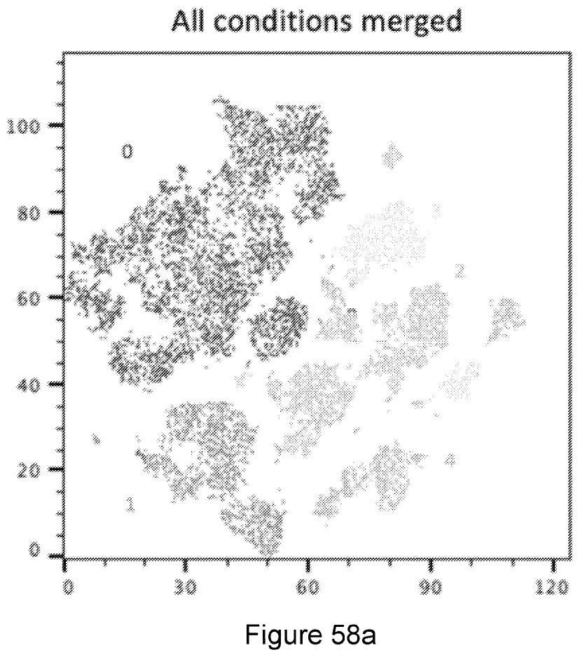

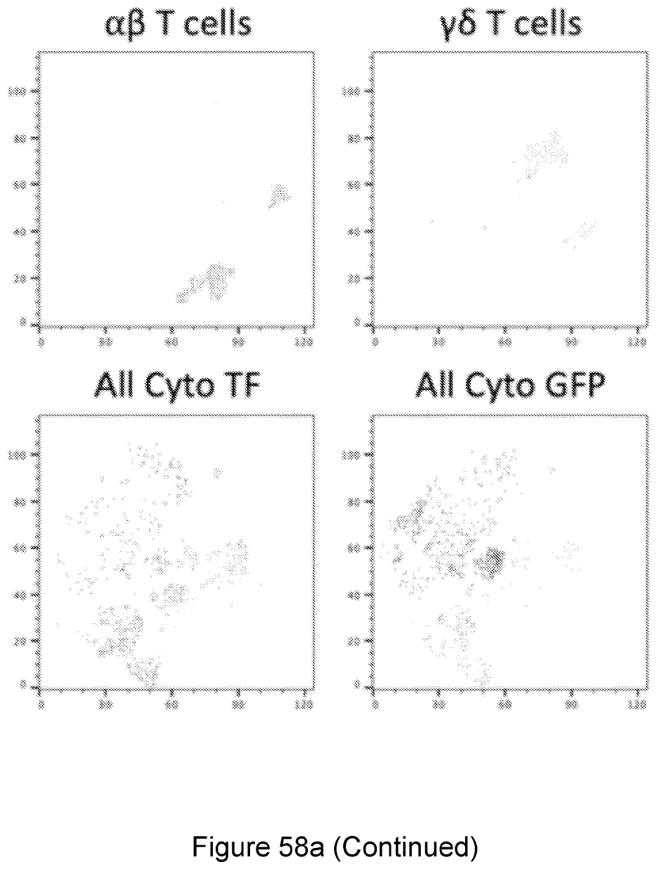

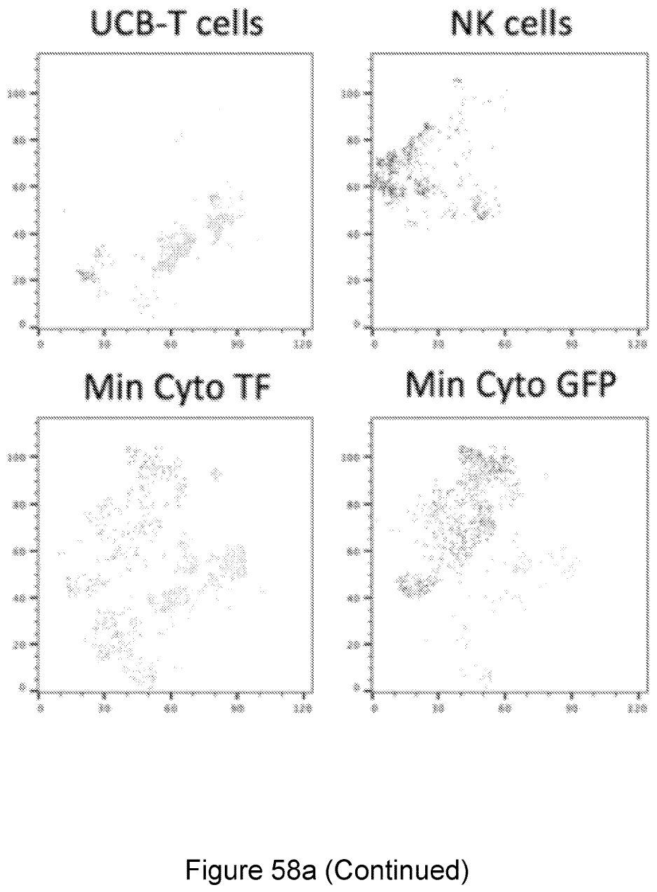

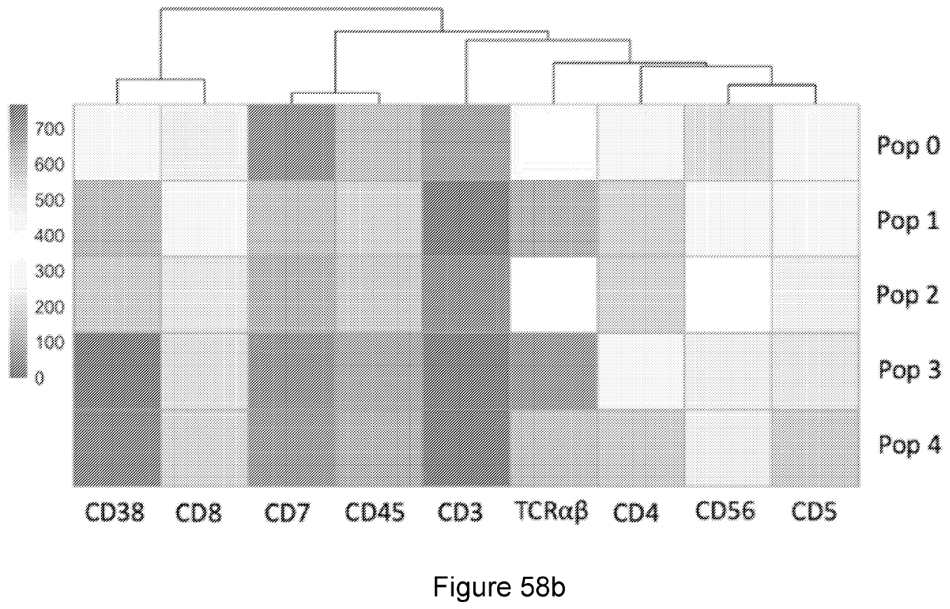

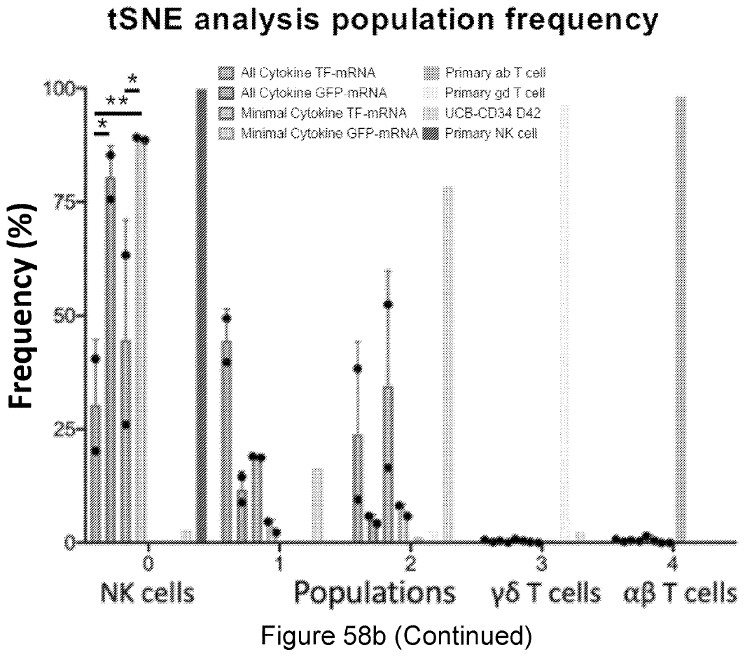

Transcription factor mRNA (TF-mRNA) (ERG, HOXA5, HOXA9, LCOR, and RUNX1) during the differentiation of iPSCs yields iPSC-HPCs with increased clonogenic output compared to GFP-mRNA treated cells. (FIGS. 52-54). The Minimal Cytokine conditions (both TF and GFP-mRNA) increased CD34+CD117+ expression in iPSC-HPCs compared to the All Cytokine conditions. Day 13 iPSC-HPCs from the Minimal Cytokine TF-mRNA condition produced 1.8× more Day 28 iPSC-T cells with a CD4+CD8+CD3+TCRαβ+phenotype compared to the All Cytokine TF-mRNA condition. Day 28 iPSC-T cells from the TF-mRNA conditions clustered more closely to UCB-T cells compared to GFP-mRNA conditions and had less of a NK phenotype. (FIGS. 55-58).

In summary, differentiating iPSCs into HPCs in the absence of hematopoietic factors, i.e., IL-3, IL-6, thrombopoietin (TPO), or an aryl hydrocarbon receptor (AHR) antagonist (also referred to as minimal cytokines), leads to HPCs with increased potential to differentiate into CD4+CD8+CD3+TCRαβ+ T cells.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. An in vitro method of generating CD34⁺ hematopoietic stem progenitor cells (HSPCs) from induced pluripotent stem cells (iPSCs) in minimal cytokine conditions comprising:
(i) contacting iPSCs with one or more mesoderm differentiation factors selected from bone morphogenetic protein 4 (BMP4), activin A, and CHIR99021;
(ii) subsequently contacting the cells of step (i) with one or more HSPC differentiation factors selected from basic fibroblast growth factor (bFGF), vascular endothelial growth factor (VEGF), stem cell factor (SCF), and SB431542;
(iii) contacting the cells of step (ii) with a plurality of nucleic acids encoding one or more transcription factors selected from ETS-related gene (ERG), ligand-dependent corepressor (LCOR), homeobox A5 (HOXA5), homeobox A9 (HOXA9), and runx family transcription factor 1 (RUNX1); and
(iv) contacting the cells of step (iii) with one or more HSPC differentiation factors selected from bFGF, VEGF, SCF, SB431542, and Y-27632 to generate CD34⁺HSPCs.

2. The method of claim 1, wherein step (i) comprises contacting the cells for about 2 days with the one or more mesoderm differentiation factors.

3. The method of claim 1, wherein step (ii) comprises contacting the cells for about 2 days with the one or more HSPC differentiation factors.

4. The method of claim 1, wherein the contacting of step (iii) comprises electroporating the cells.

5. The method of claim 1, wherein step (iv) comprises contacting the cells for about 1 day or more with the one or more HSPC differentiation factors.

6. The method of claim 1, wherein the cells of step (iv) are further cultured for about 4 days in medium with bFGF.

7. The method of claim 1, wherein the cells of step (iv) are further cultured for about 4 days in medium without cytokines or growth factors.

8. The method of claim 1, wherein the cells of step (iv) are further cultured for about 8 days in medium with bFGF.

9. The method of claim 1, wherein the cells of step (iv) are further cultured for about 8 days in medium without cytokines or growth factors.

10. The method of claim 1, wherein the cells are cultured in defined medium without serum.

11. The method of claim 1, wherein the cells are cultured in defined medium without feeder cells.

12. The method of claim 1, the method further comprising:
(v) isolating the CD34⁺ HSPCs.

13. The method of claim 1, the method comprising:
(i) contacting the cells with the mesoderm differentiation factors bone morphogenetic protein 4 (BMP 4), activin A, and CHIR99021 for about 2 days;
(ii) contacting the cells of step (i) with the HSPC differentiation factors basic fibroblast growth factor (bFGF), vascular endothelial growth factor (VEGF), stem cell factor (SCF), and SB431542 for about 2 days;
(iii) contacting the cells of step (ii) with a plurality of nucleic acids encoding the transcription factors ETS-related gene (ERG), ligand-dependent corepressor (LCOR), homeobox A5 (HOXA5), homeobox A9 (HOXA9), and runx family transcription factor 1 (RUNX1);
(iv) contacting the cells of step (iii) with the HSPC differentiation factors bFGF, VEGF, SCF, SB431542, and Y-27632 for about 1 day, and
(v) further culturing the cells of step (iv) for about 4 to about 8 days to generate CD34⁺ HSPCs.

14. The method of claim 13, the method further comprising
(vi) isolating the CD34⁺ HSPCs.

15. A population of CD34⁺ HSPCs generated by the method of any one of the preceding claims.

16. The method of claim 1 or claim 13, the method further comprising:
culturing the CD34⁺ HSPCs in the presence of one or more T cell differentiation factors selected from delta like canonical notch ligand 4 (DLL4) and vascular cell adhesion molecule (VCAM) in an amount effective to generate T cells.

17. The method of claim 16, wherein the one or more T cell differentiation factors comprises delta like canonical notch ligand 4 (DLL4) and vascular cell adhesion molecule (VCAM), and wherein the cells are not cultured in the presence of IL-3, IL-6, thrombopoietin (TPO), or an aryl hydrocarbon receptor (AHR) antagonist.

18. The method of claim 17, wherein the method produces at least six T cells for every HSPC generated by step (v) of the method.

19. A population of in vitro derived T cells generated by the method of claim 16.

20. A method of generating in vitro differentiated T cells from induced pluripotent stem cells (iPSCs) comprising:
contacting the population of cells of claim 15 with one or more T cell differentiation factors selected from delta like canonical notch ligand 4 (DLL4) and vascular cell adhesion molecule (VCAM) to generate T cells.

21. The method of claim 20, wherein the method comprises contacting the cells for about 14 days with the one or more T cell differentiation factors.

22. The method of claim 20, wherein the method comprises contacting the cells for about 28 days with the one or more T cell differentiation factors.

23. The method of any one of claims 1-14 and 20-22, wherein the iPSCs are derived from a subject or donor cell.

24. The method of any one of claims 20-23, wherein the iPSCs of step (i) are derived from donor T cells from a subject.

25. The method of claim 24, wherein the donor T cells are tumor-specific or virus-specific T cells.

26. The method of any one of claims 20-25, wherein the cells are cultured in defined medium without serum.

27. The method of any one of claims 20-26, wherein the cells are cultured in defined medium without feeder cells.

28. A method for generating in vitro differentiated T cells from induced pluripotent stem cells (iPSCs) comprising:
(i) contacting induced pluripotent stem cells (iPSCs) with the mesoderm differentiation factors bone morphogenetic protein 4 (BMP4), activin A, and CHIR99021 for about 2 days;
(ii) contacting the cells of step (i) with the HSPC differentiation factors basic fibroblast growth factor (bFGF), vascular endothelial growth factor (VEGF), stem cell factor (SCF), and SB431542 for about 2 days;
(iii) contacting the cells of step (ii) with a plurality of nucleic acids encoding the transcription factors ETS-related gene (ERG), ligand-dependent corepressor (LCOR), homeobox A5 (HOXA5), homeobox A9 (HOXA9), and runx family transcription factor 1 (RUNX1);
(iv) contacting the cells of step (iii) with the HSPC differentiation factors bFGF, VEGF, SCF, SB431542, and Y-27632 for about 1 day then further culturing the cells for about 4 to about 8 days; and
(v) culturing the cells of step (iv) in the presence of the T cell differentiation factors delta like canonical notch ligand 4 (DLL4) and/or vascular cell adhesion molecule (VCAM) to generate T cells.

29. The population of CD34+HSPCs of claim 15, wherein the cell expresses higher levels of c-kit (CD 117) as compared to cells cultured in the presence of IL-3, IL-6, thrombopoietin (TPO), or an aryl hydrocarbon receptor (AHR) antagonist.

30. An in vitro generated T cell generated by the method of any one of claims 20-28.

31. A pharmaceutical composition comprising the population of CD34⁺ hematopoietic stem progenitor cell (HSPCs) of claim 15, and a pharmaceutically acceptable carrier.

32. A pharmaceutical composition comprising the in vitro generated T cell of claim 19 or 30, and a pharmaceutically acceptable carrier.

33. A method of treating a subject in need of reconstitution of hematopoietic stem progenitor cells (HPSCs) comprising:
administering an effective amount of the pharmaceutical composition of claim 31 to a subject in need thereof to reconstitute CD34⁺ HSPCs in the subject.

34. The method of claim 33, wherein the iPSCs used to generate the CD34⁺ cells are derived from the subject.

35. A method of treating a subject in need of T cell adoptive transfer comprising:
administering an effective amount of the pharmaceutical composition of claim 32 to the subject as a T cell adoptive transfer therapy.

36. The method of claim 33, wherein the subject is in need of T cell adoptive cell transfer therapy to treat a cancer or a chronic viral infection.
